SEQUENCE LISTING (112 sequences)

SEQ ID NO: 1 - DNA, 963 bp, Artificial
  Mutated Luciferase
atgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaagccgactacaaggatgacgatgacaagtaa

SEQ ID NO: 2 - DNA, 714 bp, Artificial
  Mutated fluorescent protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgctgtgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgttcaaagaccccaacgagaagcgcgatcacatggtcctgctggagttcctgaccgccgccgggatcactgagggcatgaacgagctgtac

SEQ ID NO: 3 - DNA, 924 bp, Artificial
  Fusion protein FP-Gg9 encoding DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgctgtgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgttcaaagaccccaacgagaagcgcgatcacatggtcctgctggagttcctgaccgccgccgggatcactgagggcatgaacgagctgtacatggcccaggatctcagcgagaaggacctgttgaagatggaggtggagcagctgaagaaagaagtgaaaaacacaagaattccgatttccaaagcgggaaaggaaatcaaggagtacgtggaggcccaagcaggaaacgatccttttctcaaaggcatccctgaggacaagaatcccttcaaggagaaaggtggctgtctgataagctga

SEQ ID NO: 4 - protein, 307 aa, Artificial
  Fusion Protein FP-Gg9
MVSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYMAQDLSEKDLLKMEVEQLKKEVKNTRIPISKAGKEIKEYVEAQAGNDPFLKGIPEDKNPFKEKGGCLIS

SEQ ID NO: 5 - DNA, 924 bp, Artificial
  Fusion protein Gg9-FP encoding DNA
atggcccaggatctcagcgagaaggacctgttgaagatggaggtggagcagctgaagaaagaagtgaaaaacacaagaattccgatttccaaagcgggaaaggaaatcaaggagtacgtggaggcccaagcaggaaacgatccttttctcaaaggcatccctgaggacaagaatcccttcaaggagaaaggtggctgtctgataagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgctgtgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgttcaaagaccccaacgagaagcgcgatcacatggtcctgctggagttcctgaccgccgccgggatcactgagggcatgaacgagctgtactga

SEQ ID NO: 6 - protein, 307 aa, Artificial [sequence omitted]

SEQ ID NO: 7 - DNA, 3555 bp, Artificial
  Fusion protein GLP1R-L-G (RLG) encoding DNA
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagccacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 8 - protein, 1184 aa, Artificial
  GLP1R-L-G (RLG)  fusion protein
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCSHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNETTLYNKVAMGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 9 - DNA, 3564 bp, Artificial
  GIPR-L-G (RLG) fusion protein encoding DNA
atgactacctctccgatcctgcagctgctgctgcggctctcactgtgcgggctgctgctccagagggcggagacaggctctaaggggcagacggcgggggagctgtaccagcgctgggaacggtaccgcagggagtgccaggagaccttggcagccgcggaaccgccttcaggcctcgcctgtaacgggtccttcgatatgtacgtctgctgggactatgctgcacccaatgccactgcccgtgcgtcctgcccctggtacctgccctggcaccaccatgtggctgcaggtttcgtcctccgccagtgtggtagtgatggccaatggggactttggagagaccatacacaatgtgagaacccagagaagaatgaggcctttctggaccaaaggctcatcttggagcggttgcaggtcatgtacactgtcggctactccctgtctctcgccacactgctgctagccctgctcatcttgagtttgttcaggcggctacattgcactagaaactatatccacatcaacctgttcacgtctttcatgctgcgagctgcggccattctcagccgagaccgtctgctacctcgacctggcccctaccttggggaccaggcccttgcgctgtggaaccaggccctcgctgcctgccgcacggcccagatcgtgacccagtactgcgtgggtgccaactacacgtggctgctggtggagggcgtctacctgcacagtctcctggtgctcgtgggaggctccgaggagggccacttccgctactacctgctcctcggctggggggcccccgcgcttttcgtcattccctgggtgatcgtcaggtacctgtacgagaacacgcagtgctgggagcgcaacgaagtcaaggccatttggtggattatacggacccccatcctcatgaccatcttgattaatttcctcatttttatccgcattcttggcattctcctgtccaagctgaggacacggcaaatgcgctgccgggattaccggctgaggctggctcgctccacgctgacgctggtgcccctgctgggtgtccacgaggtggtgtttgctcccgtgacagaggaacaggcccggggcgccctgcgcttcgccaagctcggctttgagatcttcctcagctccttccagggcttcctggtcagcgtcctctactgcttcatcaacaaggaggtgcagtcggagatccgccgtggctggcaccactgccgcctgcgccgcagcctgggcgaggagcaacgccagctcccggagcgcgccttccgggccctgccctccggctccggcccgggcgaggtccccaccagccgcggcttgtcctcggggaccctcccagggcctgggaatgaggccagccgggagttggaaagttactgccacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 10 - protein, 1187 aa, Artificial
  GIPR-L-G (RLG) fusion protein
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYCHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNETTLYNKVAMGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 11 - DNA, 3402 bp, Artificial
  A2AR-L-G (RLG) fusion protein encoding DNA
atgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcccacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 12 - protein, 1133 aa, Artificial
  A2AR-L-G (RLG) fusion protein
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVSHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNETTLYNKVAMGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 13 - DNA, 3585 bp, Artificial
  GLP1R-G-L (RGL) fusion protein encoding DNA
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagccacccaacttttctatacaaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctcacaactttgtataataaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaagccgactacaaggacgacgatgacaagtaa

SEQ ID NO: 14 - protein, 1194 aa, Artificial [sequence omitted]

SEQ ID NO: 15 - DNA, 3594 bp, Artificial
  GIPR-G-L (RGL) fusion protein encoding DNA
atgactacctctccgatcctgcagctgctgctgcggctctcactgtgcgggctgctgctccagagggcggagacaggctctaaggggcagacggcgggggagctgtaccagcgctgggaacggtaccgcagggagtgccaggagaccttggcagccgcggaaccgccttcaggcctcgcctgtaacgggtccttcgatatgtacgtctgctgggactatgctgcacccaatgccactgcccgtgcgtcctgcccctggtacctgccctggcaccaccatgtggctgcaggtttcgtcctccgccagtgtggtagtgatggccaatggggactttggagagaccatacacaatgtgagaacccagagaagaatgaggcctttctggaccaaaggctcatcttggagcggttgcaggtcatgtacactgtcggctactccctgtctctcgccacactgctgctagccctgctcatcttgagtttgttcaggcggctacattgcactagaaactatatccacatcaacctgttcacgtctttcatgctgcgagctgcggccattctcagccgagaccgtctgctacctcgacctggcccctaccttggggaccaggcccttgcgctgtggaaccaggccctcgctgcctgccgcacggcccagatcgtgacccagtactgcgtgggtgccaactacacgtggctgctggtggagggcgtctacctgcacagtctcctggtgctcgtgggaggctccgaggagggccacttccgctactacctgctcctcggctggggggcccccgcgcttttcgtcattccctgggtgatcgtcaggtacctgtacgagaacacgcagtgctgggagcgcaacgaagtcaaggccatttggtggattatacggacccccatcctcatgaccatcttgattaatttcctcatttttatccgcattcttggcattctcctgtccaagctgaggacacggcaaatgcgctgccgggattaccggctgaggctggctcgctccacgctgacgctggtgcccctgctgggtgtccacgaggtggtgtttgctcccgtgacagaggaacaggcccggggcgccctgcgcttcgccaagctcggctttgagatcttcctcagctccttccagggcttcctggtcagcgtcctctactgcttcatcaacaaggaggtgcagtcggagatccgccgtggctggcaccactgccgcctgcgccgcagcctgggcgaggagcaacgccagctcccggagcgcgccttccgggccctgccctccggctccggcccgggcgaggtccccaccagccgcggcttgtcctcggggaccctcccagggcctgggaatgaggccagccgggagttggaaagttactgccacccaacttttctatacaaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctcacaactttgtataataaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaagccgactacaaggacgacgatgacaagtaa

SEQ ID NO: 16 - protein, 1197 aa, Artificial [sequence omitted]

SEQ ID NO: 17 - DNA, 6864 bp, Artificial
  A2AR-G-L (RGL) fusion protein encoding DNA
atgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcccacccaacttttctatacaaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctcacaactttgtataataaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaagccgactacaaggatgacgatgacaagtaaatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcccacccaacttttctatacaaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctcacaactttgtataataaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaagccgactacaaggatgacgatgacaagtaa

SEQ ID NO: 18 - protein, 1143 aa, Artificial [sequence omitted]

SEQ ID NO: 19 - DNA, 3528 bp, Artificial
  GLP1R-G(1-71)-L-G(82-394) (RGLG-1) fusion protein encoding DNA
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagcatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgccagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 20 - protein, 1175 aa, Artificial
  GLP1R-G(1-71)-L-G(82-394) (RGLG-1) fusion protein
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCSMGCLGNSKTEDQRNEEKAQREANKKIEKQLQKDKQVYRATHRLLLLGAGESGKSTIVKQMRILHVNGFNGEHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQTTLYNKVASNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 21 - DNA, 3537 bp, Artificial
  GIPR-G(1-71)-L-G(82-394) (RGLG-1) fusion protein encoding DNA
atgactacctctccgatcctgcagctgctgctgcggctctcactgtgcgggctgctgctccagagggcggagacaggctctaaggggcagacggcgggggagctgtaccagcgctgggaacggtaccgcagggagtgccaggagaccttggcagccgcggaaccgccttcaggcctcgcctgtaacgggtccttcgatatgtacgtctgctgggactatgctgcacccaatgccactgcccgtgcgtcctgcccctggtacctgccctggcaccaccatgtggctgcaggtttcgtcctccgccagtgtggtagtgatggccaatggggactttggagagaccatacacaatgtgagaacccagagaagaatgaggcctttctggaccaaaggctcatcttggagcggttgcaggtcatgtacactgtcggctactccctgtctctcgccacactgctgctagccctgctcatcttgagtttgttcaggcggctacattgcactagaaactatatccacatcaacctgttcacgtctttcatgctgcgagctgcggccattctcagccgagaccgtctgctacctcgacctggcccctaccttggggaccaggcccttgcgctgtggaaccaggccctcgctgcctgccgcacggcccagatcgtgacccagtactgcgtgggtgccaactacacgtggctgctggtggagggcgtctacctgcacagtctcctggtgctcgtgggaggctccgaggagggccacttccgctactacctgctcctcggctggggggcccccgcgcttttcgtcattccctgggtgatcgtcaggtacctgtacgagaacacgcagtgctgggagcgcaacgaagtcaaggccatttggtggattatacggacccccatcctcatgaccatcttgattaatttcctcatttttatccgcattcttggcattctcctgtccaagctgaggacacggcaaatgcgctgccgggattaccggctgaggctggctcgctccacgctgacgctggtgcccctgctgggtgtccacgaggtggtgtttgctcccgtgacagaggaacaggcccggggcgccctgcgcttcgccaagctcggctttgagatcttcctcagctccttccagggcttcctggtcagcgtcctctactgcttcatcaacaaggaggtgcagtcggagatccgccgtggctggcaccactgccgcctgcgccgcagcctgggcgaggagcaacgccagctcccggagcgcgccttccgggccctgccctccggctccggcccgggcgaggtccccaccagccgcggcttgtcctcggggaccctcccagggcctgggaatgaggccagccgggagttggaaagttactgcatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgccagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 22 - protein, 1178 aa, Artificial
  GIPR-G(1-71)-L-G(82-394) (RGLG-1) fusion protein
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYCMGCLGNSKTEDQRNEEKAQREANKKIEKQLQKDKQVYRATHRLLLLGAGESGKSTIVKQMRILHVNGFNGEHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQTTLYNKVASNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 23 - DNA, 3375 bp, Artificial
  A2AR-G(1-71)-L-G(82-394) (RGLG-1) fusion protein encoding DNA
atgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgccagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 24 - protein, 1124 aa, Artificial [sequence omitted]

SEQ ID NO: 25 - DNA, 55 bp, Artificial
  Primer 1
ggggacaagtttgtacaaaaaagcaggcttcgccaccatggtgagcaagggcgag

SEQ ID NO: 26 - DNA, 38 bp, Artificial
  Primer 2
ctgagatcctgggccatgtacagctcgttcatgccctc

SEQ ID NO: 27 - DNA, 38 bp, Artificial
  Primer 3
gagggcatgaacgagctgtacatggcccaggatctcag

SEQ ID NO: 28 - DNA, 50 bp, Artificial
  Primer 4
ggggaccactttgtacaagaaagctgggtatcagcttatcagacagccac

SEQ ID NO: 29 - DNA, 40 bp, Artificial
  Primer 5
gaaaggtggctgtctgataagcatggtgagcaagggcgag

SEQ ID NO: 30 - DNA, 53 bp, Artificial
  Primer 6
ggggaccactttgtacaagaaagctgggtatcagtacagctcgttcatgccct

SEQ ID NO: 31 - DNA, 57 bp, Artificial
  Primer 7
ggggacaagtttgtacaaaaaagcaggcttcgccaccatggcccaggatctcagcga

SEQ ID NO: 32 - DNA, 40 bp, Artificial
  Primer 8
ctcgcccttgctcaccatgcttatcagacagccacctttc

SEQ ID NO: 33 - DNA, 21 bp, Artificial
  Primer 9
agtctcagctgcaggaggcca

SEQ ID NO: 34 - DNA, 19 bp, Artificial
  Primer 10
gtcctgaactccccgccat

SEQ ID NO: 35 - DNA, 69 bp, Artificial
  Primer 11
ggggacaagtttgtacaaaaaagcaggcttcgaaggagatagaaccatggccggcgcccccggcccgct

SEQ ID NO: 36 - DNA, 52 bp, Artificial
  Primer 12
ggggacaactttgtatagaaaagttgggtggctgcaggaggcctggcaagtg

SEQ ID NO: 37 - DNA, 51 bp, Artificial
  Primer 13
ggggacaacttttctatacaaagttgccatgaccagcaaggtgtacgaccc

SEQ ID NO: 38 - DNA, 49 bp, Artificial
  Primer 14
ggggacaactttattatacaaagttgtctcgttcttcagcactctctcc

SEQ ID NO: 39 - DNA, 17 bp, Artificial
  Primer 15
atgggctgcctcgggaa

SEQ ID NO: 40 - DNA, 20 bp, Artificial
  Primer 16
ttagagcagctcgtactgac

SEQ ID NO: 41 - DNA, 46 bp, Artificial
  Primer 17
ggggacaactttgtataataaagttgccatgggctgcctcgggaac

SEQ ID NO: 42 - DNA, 50 bp, Artificial
  Primer 18
ggggaccactttgtacaagaaagctgggtattagagcagctcgtactgac

SEQ ID NO: 43 - DNA, 18 bp, Artificial
  Primer 19
atcccgccccctagcagt

SEQ ID NO: 44 - DNA, 18 bp, Artificial
  Primer 20
acgaaccagacccttcgc

SEQ ID NO: 45 - DNA, 72 bp, Artificial
  Primer 21
ggggacaagtttgtacaaaaaagcaggcttcgaaggagatagaaccatgactacctctccgatcctgcagct

SEQ ID NO: 46 - DNA, 48 bp, Artificial
  Primer 22
ggggacaactttgtatagaaaagttgggtggcagtaactttccaactc

SEQ ID NO: 47 - DNA, 20 bp, Artificial
  Primer 23
atgcccatcatgggctcctc

SEQ ID NO: 48 - DNA, 20 bp, Artificial
  Primer 24
tcaggacactcctgctccat

SEQ ID NO: 49 - DNA, 52 bp, Artificial
  Primer 25
ggggacaagtttgtacaaaaaagcaggcttcatgcccatcatgggctcctcg

SEQ ID NO: 50 - DNA, 51 bp, Artificial
  Primer 26
ggggacaactttgtatagaaaagttgggtgggacactcctgctccatcctg

SEQ ID NO: 51 - DNA, 45 bp, Artificial
  Primer 27
ggggacaacttttctatacaaagttgccatgggctgcctcgggaa

SEQ ID NO: 52 - DNA, 45 bp, Artificial
  Primer 28
ggggacaactttattatacaaagttgtgagcagctcgtactgacg

SEQ ID NO: 53 - DNA, 46 bp, Artificial
  Primer 29
ggggacaactttgtataataaagttgccatgaccagcaaggtgtac

SEQ ID NO: 54 - DNA, 50 bp, Artificial
  Primer 30
ggggaccactttgtacaagaaagctgggtattacttgtcatcgtcgtcct

SEQ ID NO: 55 - DNA, 61 bp, Artificial
  Primer 31
ggggacaagtttgtacaaaaaagcaggcttcgccaccatgggctgcctcgggaacagtaag

SEQ ID NO: 56 - DNA, 54 bp, Artificial
  Primer 32
ggggacaactttgtatagaaaagttgggtgctctccattaaacccattaacatg

SEQ ID NO: 57 - DNA, 51 bp, Artificial
  Primer 33
ggggacaactttattatacaaagttgtttgctcgttcttcagcactctctc

SEQ ID NO: 58 - DNA, 51 bp, Artificial; identical to SEQ ID NO: 37
  Primer 34

SEQ ID NO: 59 - DNA, 50 bp, Artificial
  Primer 35
ggggacaactttgtataataaagttgccagcaacagcgatggtgagaagg

SEQ ID NO: 60 - DNA, 50 bp, Artificial; identical to SEQ ID NO: 42
  Primer 36

SEQ ID NO: 61 - DNA, 61 bp, Artificial
  Primer 37
ggggacaagtttgtacaaaaaagcaggcttcgccaccatggccggcgcccccggcccgctg

SEQ ID NO: 62 - DNA, 32 bp, Artificial
  Primer 38
ttcccgaggcagcccatgctgcaggaggcctg

SEQ ID NO: 63 - DNA, 32 bp, Artificial
  Primer 39
caggcctcctgcagcatgggctgcctcgggaa

SEQ ID NO: 64 - DNA, 36 bp, Artificial
  Primer 40
ttcccgaggcagcccatgcagtaactttccaactcc

SEQ ID NO: 65 - DNA, 31 bp, Artificial
  Primer 41
tggaaagttactgcatgggctgcctcgggaa

SEQ ID NO: 66 - DNA, 52 bp, Artificial; identical to SEQ ID NO: 49
  Primer 42

SEQ ID NO: 67 - DNA, 33 bp, Artificial 44
  Primer 43
ttcccgaggcagcccatggacactcctgctcca

SEQ ID NO: 68 - DNA, 33 bp, Artificial
  Primer 44
tggagcaggagtgtccatgggctgcctcgggaa

SEQ ID NO: 69 - DNA, 50 bp, Artificial; identical to SEQ ID NO: 42
  Primer 45

SEQ ID NO: 70 - DNA, 52 bp, Artificial; identical to SEQ ID NO: 49
  Primer 46

SEQ ID NO: 71 - DNA, 50 bp, Artificial; identical to SEQ ID NO: 42
  Primer 47

SEQ ID NO: 72 - protein, 9 aa, Artificial
  Linker motif 1 in fusion proteins
HPTFLYKVG

SEQ ID NO: 73 - protein, 8 aa, Artificial
  Linker motif 2 in fusion proteins
TTLYNKVA

SEQ ID NO: 74 - DNA, 24 bp, Artificial
  Primer 48
atgggatgtactctgagcgcagag

SEQ ID NO: 75 - DNA, 18 bp, Artificial
  Primer 49
tcagtacaagccgcagcc

SEQ ID NO: 76 - DNA, 70 bp, Artificial
  Primer 50
ggggacaagtttgtacaaaaaagcaggctctggaggcggaggatctatgggatgtactctgagcgcagag

SEQ ID NO: 77 - DNA, 73 bp, Artificial
  Primer 51
ggggacaactttgtatagaaaagttgggtgagatccgcctcctccagacaaagtgtccatggcccggacgatg

SEQ ID NO: 78 - DNA, 70 bp, Artificial
  Primer 52
ggggacaactttgtataataaagttgcctctggaggaggaggctctggcatcgaatatggtgataaggag

SEQ ID NO: 79 - DNA, 48 bp, Artificial
  Primer 53
ggggaccactttgtacaagaaagctgggtatcagtacaagccgcagcc

SEQ ID NO: 80 - DNA, 58 bp, Artificial
  Primer 54
ggggacaagtttgtacaaaaaagcaggcttcgccaccatggccaacttcacacctgtc

SEQ ID NO: 81 - DNA, 60 bp, Artificial
  Primer 55
agtacatcccatagatcctccgcctccagacctggcagtgccgatgttccgatactggca

SEQ ID NO: 82 - DNA, 60 bp, Artificial
  Primer 56
tgccagtatcggaacatcggcactgccaggtctggaggcggaggatctatgggatgtact

SEQ ID NO: 83 - DNA, 3540 bp, Artificial
  hM4R-Go(1-91)-L-Go(92-354) (RGLG-2)) fusion protein encoding DNA
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactacgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctgccttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctagccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggtctggaggcggaggatctatgggatgtactctgagcgcagaggagagagccgccctcgagcggagcaaggcgattgagaaaaacctcaaagaggatggcatcagcgccgccaaagacgtgaaattactcctgctcggggctggagaatcaggaaaaagcaccattgtgaagcagatgaagatcatccatgaagatggcttctccggagaagacgtgaaacagtacaagcctgttgtctacagcaacactatccagtccctggcagccatcgtccgggccatggacactttgtctggaggaggcggatctcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgcctctggaggaggaggctctggcatcgaatatggtgataaggagagaaaggctgacgccaagatggtgtgtgatgtggtgagtcggatggaagacaccgagcccttctctgcagagctgctttctgccatgatgcggctctggggcgactcaggaatccaagagtgcttcaaccggtcccgggagtatcagctcaacgactctgccaaatactacctggacagcctggatcggattggggccgccgactaccagcccactgagcaggacatcctccgaaccagggtcaaaaccactggcatcgtagaaacccacttcacattcaagaacctccacttcaggctgtttgacgtcggaggccagcgatctgaacgcaagaagtggatccattgcttcgaggacgtcacggccatcattttctgtgtcgcgctcagcggctatgaccaggtgctccacgaagacgaaaccacgaaccgcatgcacgagtctctcatgctcttcgactccatctgtaacaacaagttcttcatcgatacctccatcattctcttcctcaacaagaaagatctctttggcgagaagatcaagaagtcacctttgaccatctgctttcctgaatacacaggccccaatacctatgaagacgcagccgcctacatccaagcacaatttgaaagcaaaaaccgctcacccaacaaagaaatatattgtcacatgacttgtgccacagacacgaataacatccaggtggtgttcgacgccgtcaccgacatcatcattgccaacaacctccggggctgcggcttgtactga

SEQ ID NO: 84 - protein, 1179 aa, Artificial
  hM4R-Go(1-91)-L-Go(92-354) (RGLG-2)) fusion protein
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDYVVSNASVMNLLIISFDRYFCVTKPLTYPARRTTKMAGLMIAAAWVLSFVLWAPAILFWQFVVGKRTVPDNQCFIQFLSNPAVTFGTAIAAFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTARSGGGGSMGCTLSAEERAALERSKAIEKNLKEDGISAAKDVKLLLLGAGESGKSTIVKQMKIIHEDGFSGEDVKQYKPVVYSNTIQSLAAIVRAMDTLSGGGGSHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQTTLYNKVASGGGGSGIEYGDKERKADAKMVCDVVSRMEDTEPFSAELLSAMMRLWGDSGIQECFNRSREYQLNDSAKYYLDSLDRIGAADYQPTEQDILRTRVKTTGIVETHFTFKNLHFRLFDVGGQRSERKKWIHCFEDVTAIIFCVALSGYDQVLHEDETTNRMHESLMLFDSICNNKFFIDTSIILFLNKKDLFGEKIKKSPLTICFPEYTGPNTYEDAAAYIQAQFESKNRSPNKEIYCHMTCATDTNNIQVVFDAVTDIIIANNLRGCGLY

SEQ ID NO: 85 - protein, 6 aa, Artificial
  Linker
SGGGGS

SEQ ID NO: 86 - DNA, 3498 bp, Artificial
  hM1R-Gq[1-124]-L-Gq(125-359) (RGLG-3) fusion protein encoding DNA
atgaacacttcagccccacctgctgtcagccccaacatcaccgtcctggcaccaggaaagggtccctggcaagtggccttcattgggatcaccacgggcctcctgtcgctagccacagtgacaggcaacctgctggtactcatctctttcaaggtcaacacggagctcaagacagtcaataactacttcctgctgagcctggcctgtgctgacctcatcatcggtaccttctccatgaacctctataccacgtacctgctcatgggccactgggctctgggcacgctggcttgtgacctctggctggccctggactatgtggccagcaatgcctccgtcatgaatctgctgctcatcagctttgaccgctacttctccgtgactcggcccctgagctaccgtgccaagcgcacaccccgccgggcagctctgatgatcggcctggcctggctggtttcctttgtgctctgggccccagccatcctcttctggcagtacctggtaggggagcggacagtgctagctgggcagtgctacatccagttcctctcccagcccatcatcacctttggcacagccatggctgccttctacctccctgtcacagtcatgtgcacgctctactggcgcatctaccgggagacagagaaccgagcacgggagctggcagcccttcagggctccgagacgccaggcaaagggggtggcagcagcagcagctcagagaggtctcagccaggggctgagggctcaccagagactcctccaggccgctgctgtcgctgctgccgggcccccaggctgctgcaggcctacagctggaaggaagaagaggaagaggacgaaggctccatggagtccctcacatcctcagagggagaggagcctggctccgaagtggtgatcaagatgccaatggtggaccccgaggcacaggcccccaccaagcagcccccacggagctccccaaatacagtcaagaggccgactaagaaagggcgtgatcgagctggcaagggccagaagccccgtggaaaggagcagctggccaagcggaagaccttctcgctggtcaaggagaagaaggcggctcggaccctgagtgccatcctcctggccttcatcctcacctggacaccgtacaacatcatggtgctggtgtccaccttctgcaaggactgtgttcccgagaccctgtgggagctgggctactggctgtgctacgtcaacagcaccatcaaccccatgtgctacgcactctgcaacaaagccttccgggacacctttcgcctgctgctgctttgccgctgggacaagagacgctggcgcaagatccccaagcgccctggctccgtgcaccgcactccctcccgccaatgctctggaggcggaggatctatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgctttttctggaggaggcggatctcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgcctctggaggaggaggctctgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaa

SEQ ID NO: 87 - protein, 1165 aa, Artificial
  hM1R-Gq[1-124]-L-Gq(125-359) (RGLG-3) fusion protein
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQCSGGGGSMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFSGGGGSHPTFLYKVAMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQTTLYNKVASGGGGSENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 88 - DNA, 3501 bp, Artificial
  hM2R-Go[1-91]-L-Go(92-354) (RGLG-2) fusion protein encoding DNA
atgaataactcaacaaactcctctaacaatagcctggctcttacaagtccttataagacatttgaagtggtgtttattgtcctggtggctggatccctcagtttggtgaccattatcgggaacatcctagtcatggtttccattaaagtcaaccgccacctccagaccgtcaacaattactttttattcagcttggcctgtgctgaccttatcataggtgttttctccatgaacttgtacaccctctacactgtgattggttactggcctttgggacctgtggtgtgtgacctttggctagccctggactatgtggtcagcaatgcctcagttatgaatctgctcatcatcagctttgacaggtacttctgtgtcacaaaacctctgacctacccagtcaagcggaccacaaaaatggcaggtatgatgattgcagctgcctgggtcctctctttcatcctctgggctccagccattctcttctggcagttcattgtaggggtgagaactgtggaggatggggagtgctacattcagtttttttccaatgctgctgtcacctttggtacggctattgcagccttctatttgccagtgatcatcatgactgtgctatattggcacatatcccgagccagcaagagcaggataaagaaggacaagaaggagcctgttgccaaccaagaccccgtttctccaagtctggtacaaggaaggatagtgaagccaaacaataacaacatgcccagcagtgacgatggcctggagcacaacaaaatccagaatggcaaagcccccagggatcctgtgactgaaaactgtgttcagggagaggagaaggagagctccaatgactccacctcagtcagtgctgttgcctctaatatgagagatgatgaaataacccaggatgaaaacacagtttccacttccctgggccattccaaagatgagaactctaagcaaacatgcatcagaattggcaccaagaccccaaaaagtgactcatgtaccccaactaataccaccgtggaggtagtggggtcttcaggtcagaatggagatgaaaagcagaatattgtagcccgcaagattgtgaagatgactaagcagcctgcaaaaaagaagcctcctccttcccgggaaaagaaagtcaccaggacaatcttggctattctgttggctttcatcatcacttgggccccatacaatgtcatggtgctcattaacaccttttgtgcaccttgcatccccaacactgtgtggacaattggttactggctttgttacatcaacagcactatcaaccctgcctgctatgcactttgcaatgccaccttcaagaagacctttaaacaccttctcatgtgtcattataagaacataggcgctacaaggtctggaggcggaggatctatgggatgtactctgagcgcagaggagagagccgccctcgagcggagcaaggcgattgagaaaaacctcaaagaggatggcatcagcgccgccaaagacgtgaaattactcctgctcggggctggagaatcaggaaaaagcaccattgtgaagcagatgaagatcatccatgaagatggcttctccggagaagacgtgaaacagtacaagcctgttgtctacagcaacactatccagtccctggcagccatcgtccgggccatggacactttgtctggaggaggcggatctcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgcctctggaggaggaggctctggcatcgaatatggtgataaggagagaaaggctgacgccaagatggtgtgtgatgtggtgagtcggatggaagacaccgagcccttctctgcagagctgctttctgccatgatgcggctctggggcgactcaggaatccaagagtgcttcaaccggtcccgggagtatcagctcaacgactctgccaaatactacctggacagcctggatcggattggggccgccgactaccagcccactgagcaggacatcctccgaaccagggtcaaaaccactggcatcgtagaaacccacttcacattcaagaacctccacttcaggctgtttgacgtcggaggccagcgatctgaacgcaagaagtggatccattgcttcgaggacgtcacggccatcattttctgtgtcgcgctcagcggctatgaccaggtgctccacgaagacgaaaccacgaaccgcatgcacgagtctctcatgctcttcgactccatctgtaacaacaagttcttcatcgatacctccatcattctcttcctcaacaagaaagatctctttggcgagaagatcaagaagtcacctttgaccatctgctttcctgaatacacaggccccaatacctatgaagacgcagccgcctacatccaagcacaatttgaaagcaaaaaccgctcacccaacaaagaaatatattgtcacatgacttgtgccacagacacgaataacatccaggtggtgttcgacgccgtcaccgacatcatcattgccaacaacctccggggctgcggcttgtactga

SEQ ID NO: 89 - protein, 1166 aa, Artificial [sequence omitted]

SEQ ID NO: 90 - DNA, 3195 bp, Artificial
  GHSR-L-G (RLG) fusion protein encoding DNA
atgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggtatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatcttcttcttccttcctgtcttctgtctcacggtcctctacagtctcatcggcaggaagctgtggcggaggaggcgcggcgatgctgtcgtgggtgcctcgctcagggaccagaaccacaagcaaaccgtgaaaatgctggctgtagtggtgtttgccttcatcctctgctggctccccttccacgtagggcgatatttattttccaaatcctttgagcctggctccttggagattgctcagatcagccagtactgcaacctcgtgtcctttgtcctcttctacctcagtgctgccatcaaccccattctgtacaacatcatgtccaagaagtaccgggtggcagtgttcagacttctgggattcgaacccttctcccagagaaagctctccactctgaaagatgaaagttctcgggcctggacagaatctagtattaatacatctggaggaggcggatctcacccaacttttctatacaaagttggaatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgcctctggaggcggaggatctatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaa

SEQ ID NO: 91 - protein, 1064 aa, Artificial [sequence omitted]

SEQ ID NO: 92 - DNA, 3825 bp, Artificial
  Glp2R-L-G (RLG) fusion protein encoding DNA
atgaagctgggatcgagcagggcagggcctgggagaggaagcgcgggactcctgcctggcgtccacgagctgcccatgggcatccctgccccctgggggaccagtcctctctccttccacaggaagtgctctctctgggcccctgggaggcccttcctcactctggtcctgctggtttccatcaagcaagttacaggatccctccttgaggaaacgactcggaagtgggctcagtacaaacaggcatgtctgagagacttactcaaggaaccttctggcatattttgtaacgggacatttgatcagtacgtgtgttggcctcattcttctcctggaaatgtctctgtaccctgcccttcatacttaccttggtggagtgaagagagctcaggaagggcctacagacactgcttggctcaggggacttggcagacgatagagaacgccacggatatttggcaggatgactccgaatgctccgagaaccacagcttcaagcaaaacgtggatcgttatgccttgctgtcaaccttgcagctgatgtacaccgtgggatactccttctctcttatctccctcttcctggctctcaccctcctcttgtttcttcgaaaactccactgcacgcgcaactacatccacatgaacttgtttgcttctttcatcctgagaaccctggctgtactggtgaaggacgtcgtcttctacaactcttactccaagaggcctgacaatgagaatgggtggatgtcctacctgtcagagatgtccacctcctgccgctcagtccaggttctcttgcattactttgtgggtgccaattacttatggctgctggtcgaaggcctctacctccacacgctgctggagcccacagtgcttcctgagaggcggctgtggcccagatacctgctgttgggttgggccttccctgtgctatttgttgtaccctggggtttcgcccgtgcacacctggagaacacagggtgctggacaacaaatgggaataagaaaatctggtggatcatccgaggacccatgatgctctgtgtaacagtcaatttcttcatcttcctgaaaattctcaagcttctcatttctaagctcaaagctcatcaaatgtgcttcagagattataaatacagattggcaaaatcaacactggtcctcattcctttattgggcgttcatgagatcctcttctctttcatcactgatgatcaagttgaaggatttgcaaaacttatacgacttttcattcagttgacactgagctcctttcatgggttcctggtggccttgcagtatggttttgccaatggagaggtgaaggctgagctgcggaaatactgggtccgcttcttgctagcccgccactcaggctgcagagcctgtgtcctggggaaggacttccggttcctaggaaaatgtcccaagaagctctcggaaggagatggcgctgagaagcttcggaagctgcagccctcacttaacagtgggcggctcctacatctagccatgcgaggtcttggggagctgggcgcccagccccaacaggaccatgcacgctggccccggggcagcagcctgtccgagtgcagtgagggggatgtcaccatggccaacaccatggaggagattctggaagagagtgagatccacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 93 - protein, 1274 aa, Artificial [sequence omitted]

SEQ ID NO: 94 - DNA, 3597 bp, Artificial
  GlucR-L-G (RLG) fusion protein encoding DNA
atgcccccctgccagccacagcgacccctgctgctgttgctgctgctgctggcctgccagccacaggtcccctccgctcaggtgatggacttcctgtttgagaagtggaagctctacggtgaccagtgtcaccacaacctgagcctgctgccccctcccacggagctggtgtgcaacagaaccttcgacaagtattcctgctggccggacacccccgccaataccacggccaacatctcctgcccctggtacctgccttggcaccacaaagtgcaacaccgcttcgtgttcaagagatgcgggcccgacggtcagtgggtgcgtggaccccgggggcagccttggcgtgatgcctcccagtgccagatggatggcgaggagattgaggtccagaaggaggtggccaagatgtacagcagcttccaggtgatgtacacagtgggctacagcctgtccctgggggccctgctcctcgccttggccatcctggggggcctcagcaagctgcactgcacccgcaatgccatccacgcgaatctgtttgcgtccttcgtgctgaaagccagctccgtgctggtcattgatgggctgctcaggacccgctacagccagaaaattggcgacgacctcagtgtcagcacctggctcagtgatggagcggtggctggctgccgtgtggccgcggtgttcatgcaatatggcatcgtggccaactactgctggctgctggtggagggcctgtacctgcacaacctgctgggcctggccaccctccccgagaggagcttcttcagcctctacctgggcatcggctggggtgcccccatgctgttcgtcgtcccctgggcagtggtcaagtgtctgttcgagaacgtccagtgctggaccagcaatgacaacatgggcttctggtggatcctgcggttccccgtcttcctggccatcctgatcaacttcttcatcttcgtccgcatcgttcagctgctcgtggccaagctgcgggcacggcagatgcaccacacagactacaagttccggctggccaagtccacgctgaccctcatccctctgctgggcgtccacgaagtggtcttcgccttcgtgacggacgagcacgcccagggcaccctgcgctccgccaagctcttcttcgacctcttcctcagctccttccagggcctgctggtggctgtcctctactgcttcctcaacaaggaggtgcagtcggagctgcggcggcgttggcaccgctggcgcctgggcaaagtgctatgggaggagcggaacaccagcaaccacagggcctcatcttcgcccggccacggccctcccagcaaggagctgcagtttgggaggggtggtggcagccaggattcatctgcggagacccccttggctggtggcctccctagattggctgagagccccttccacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 95 - protein, 1198 aa, Artificial [sequence omitted]

SEQ ID NO: 96 - DNA, 4719 bp, Artificial
  hmGluR2-Go[1-91]-L-Go(92-354) (RGLG-2) fusion protein encoding DNA
atgggatcgctgcttgcgctcctggcactgctgctgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcctgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttctggaggcggaggatctatgggatgtactctgagcgcagaggagagagccgccctcgagcggagcaaggcgattgagaaaaacctcaaagaggatggcatcagcgccgccaaagacgtgaaattactcctgctcggggctggagaatcaggaaaaagcaccattgtgaagcagatgaagatcatccatgaagatggcttctccggagaagacgtgaaacagtacaagcctgttgtctacagcaacactatccagtccctggcagccatcgtccgggccatggacactttgtctggaggaggcggatctcacccaacttttctatacaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagcaaacaactttgtataataaagttgcctctggaggaggaggctctggcatcgaatatggtgataaggagagaaaggctgacgccaagatggtgtgtgatgtggtgagtcggatggaagacaccgagcccttctctgcagagctgctttctgccatgatgcggctctggggcgactcaggaatccaagagtgcttcaaccggtcccgggagtatcagctcaacgactctgccaaatactacctggacagcctggatcggattggggccgccgactaccagcccactgagcaggacatcctccgaaccagggtcaaaaccactggcatcgtagaaacccacttcacattcaagaacctccacttcaggctgtttgacgtcggaggccagcgatctgaacgcaagaagtggatccattgcttcgaggacgtcacggccatcattttctgtgtcgcgctcagcggctatgaccaggtgctccacgaagacgaaaccacgaaccgcatgcacgagtctctcatgctcttcgactccatctgtaacaacaagttcttcatcgatacctccatcattctcttcctcaacaagaaagatctctttggcgagaagatcaagaagtcacctttgaccatctgctttcctgaatacacaggccccaatacctatgaagacgcagccgcctacatccaagcacaatttgaaagcaaaaaccgctcacccaacaaagaaatatattgtcacatgacttgtgccacagacacgaataacatccaggtggtgttcgacgccgtcaccgacatcatcattgccaacaacctccggggctgcggcttgtactga

SEQ ID NO: 97 - protein, 1572 aa, Artificial [sequence omitted]

SEQ ID NO: 98 - DNA, 3369 bp, Artificial
  Ox1R-L-G (RLG) fusion protein encoding DNA
atggagccctcagccaccccaggggcccagatgggggtcccccctggcagcagagagccgtcccctgtgcctccagactatgaagatgagtttctccgctatctgtggcgcgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcacggaggaagacagccaagatgctgatggtggtgctgctggtcttcgccctctgctacctgcccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggcaaattccgggagcagtttaaggctgccttctcctgctgcctgcctggcctgggtccctgcggctctctgaaggcccctagtccccgctcctctgccagccacaagtccttgtccttgcagagccgatgctccatctccaaaatctctgagcatgtggtgctcaccagcgtcaccacagtgctgccctctggaggaggcggatctacaactttgtatacaaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgcctctggaggcggaggatctatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaa

SEQ ID NO: 99 - protein, 1122 aa, Artificial [sequence omitted]

SEQ ID NO: 100 - DNA, 3426 bp, Artificial
  Ox2R-L-G (RLG) fusion protein encoding DNA
atgtccggcaccaaattggaggactcccccccttgtcgcaactggtcatctgcttcggagctgaatgaaactcaagagccctttttaaaccccaccgactatgacgacgaggaattcctgcggtacctgtggagggaatacctgcacccgaaagaatatgagtgggtcctgatcgccgggtacatcatcgtgttcgtcgtggctctcattgggaacgtcctggtttgtgtggcagtgtggaagaaccaccacatgaggacggtaaccaactacttcatagtcaatctttctctggctgatgtgctcgtgaccatcacctgccttccagccacactggtcgtggatatcactgagacctggttttttggacagtccctttgcaaagtgattccttatctacagaccgtgtcggtgtctgtgtctgtcctcacactgagctgtatcgccttggatcggtggtatgcaatctgtcaccctttgatgtttaagagcacagcaaagcgggcccgtaacagcattgtcatcatctggattgtctcctgcattataatgattcctcaggccatcgtcatggagtgcagcaccgtgttcccaggcttagccaataaaaccaccctctttacggtgtgtgatgagcgctggggtggtgaaatttatcccaagatgtaccacatctgtttctttctggtgacatacatggcaccactgtgtctcatggtgttggcttatctgcaaatatttcgcaaactctggtgtcgacagatccctggaacatcatctgtagttcagagaaaatggaagcccctgcagcctgtttcacagcctcgagggccaggacagccaacgaagtcccggatgagcgctgtggcggctgaaataaagcagatccgagccagaaggaaaacagcccggatgttgatggttgtgcttttggtatttgcaatttgctatctaccaattagcatcctcaatgtgctaaagagagtatttgggatgtttgcccatactgaagacagagagactgtgtatgcctggtttaccttttcacactggcttgtatatgccaatagtgctgcgaatccaattatttataattttctcagtggaaaatttcgagaggaatttaaagctgcgttttcttgctgttgccttggagttcaccatcgccaggaggatcggctcaccaggggacgaactagcacagagagccggaagtccttgaccactcaaatcagcaactttgataacatatcaaaactttctgaacaagttgtgctcactagcataagcacactcccagcagccaatggagcaggaccacttcaaaactggtctggaggaggcggatctacaactttgtatacaaaagttgccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccaccagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggagctgcactggccttccactacgcctacgagcaccaggacagaatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtcctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagagcgaccccggcttcttcagcaacgccatcgtggagggcgccaagaagttccccaacaccgagttcgtgaaggtgaagggcctgcactttctccaggaagacgcccccgacgagatgggcaagtacatcaagagcttcgtggagagagtgctgaagaacgagacaactttgtataataaagttgcctctggaggcggaggatctatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaa

SEQ ID NO: 101 - protein, 1141 aa, Artificial [sequence omitted]

SEQ ID NO: 102 - protein, 74 aa, Homo sapiens
MPVINIEDLTEKDKLKMEVDQLKKEVTLERMLVSKCCEEVRDYVEERSGEDPLVKGIPEDKNPFKELKGGCVIS

SEQ ID NO: 103 - protein, 71 aa, Homo sapiens
MASNNTASIAQARKLVEQLKMEANIDRIKVSKAAADLMAYCEAHAKEDPLLTPVPASENPFREKKFFCAIL

SEQ ID NO: 104 - protein, 75 aa, Homo sapiens
MKGETPVNSTMSIGQARKMVEQLKIEASLCRIKVSKAAADLMTYCDAHACEDPLITPVPTSENPFREKKFFCALL

SEQ ID NO: 105 - protein, 75 aa, Homo sapiens
MKEGMSNNSTTSISQARKAVEQLKMEACMDRVKVSQAAADLLAYCEAHVREDPLIIPVPASENPFREKKFFCTIL

SEQ ID NO: 106 - protein, 68 aa, Homo sapiens
MSGSSSVAAMKKVVQQLRLEAGLNRVKVSQAAADLKQFCLQNAQHDPLLTGVSSSTNPFRPQKVCSFL

SEQ ID NO: 107 - protein, 68 aa, Homo sapiens
MSATNNIAQARKLVEQLRIEAGIERIKVSKAASDLMSYCEQHARNDPLLVGVPASENPFKDKKPCIIL

SEQ ID NO: 108 - protein, 70 aa, Homo sapiens
MSNNMAKIAEARKTVEQLKLEVNIDRMKVSQAAAELLAFCETHAKDDPLVTPVPAAENPFRDKRLFCVLL

SEQ ID NO: 109 - protein, 68 aa, Homo sapiens
MSSGASASALQRLVEQLKLEAGVERIKVSQAAAELQQYCMQNACKDALLVGVPAGSNPFREPRSCALL

SEQ ID NO: 110 - protein, 73 aa, Homo sapiens
MPALHIEDLPEKEKLKMEVEQLRKEVKLQRQQVSKCSEEIKNYIEERSGEDPLVKGIPEDKNPFKEKGSCVIS

SEQ ID NO: 111 - protein, 72 aa, Homo sapiens
MSSKTASTNNIAQARRTVQQLRLEASIERIKVSKASADLMSYCEEHARSDPLLIGIPTSENPFKDKKTCIIL

SEQ ID NO: 112 - protein, 67 aa, Homo sapiens
MEEWDVPQMKKEVESLKYQLAFQREMASKTIPELLKWIEDGIPKDPFLNPDLMKNNPWVEKGKCTIL

(19) United States
(12) Patent Application Publication
(10) Pub. No.: US 2013/0035256 A1
(43) Pub. Date: Feb. 7, 2013

(54) Chimeric Polypeptides Useful in Proximal and Dynamic High-Throughput Screening Methods

(71) Applicants: Tina Gjoni, Plan-les-Ouates (CH); Dominik Schelshorn, Plan-les-Ouates (CH); Cornelia Hampe, Plan-les-Ouates (CH); Aurelie Gouiller, Plan-les-Ouates (CH); Robert Lutjens, Plan-les-Ouates (CH)

(72) Inventors: Tina Gjoni, Plan-les-Ouates (CH); Dominik Schelshorn, Plan-les-Ouates (CH); Cornelia Hampe, Plan-les-Ouates (CH); Aurelie Gouiller, Plan-les-Ouates (CH); Robert Lutjens, Plan-les-Ouates (CH)

(73) Assignee: ADDEX PHARMA SA, (CH)

(21) Appl. No.: 13/640,332

(22) PCT Filed: Apr. 20, 2011
(86) PCT No.: PCT/EP11/56389

§ 371 Date: Oct. 10, 2012

(30) Foreign Application Priority Data
Apr. 20, 2010 (EP) 10160519.4

(51) Int. Cl. C40B 30/04; C07K 14/705; C12N 9/96; C12N 5/10; C40B 30/06; C12N 15/62

(52) U.S. Cl. 506/9; 506/10; 536/23.4; 435/188; 435/369; 530/350

(57) ABSTRACT

The present invention provides a method of high-throughput screening (HTS) of active agents of a cell-surface G-Protein coupled receptor (GPCR) or another target receptor of interest. The method uses a non-invasive, sensitive reporting system that is proximal to the target of interest, combined with a dynamic, automated screening procedure so as to detect orthosteric ligands, such as agonists and antagonists, but is also suitable to detect allosteric or low affinity active agents of a GPCR and possibly other disease-related receptors.

### TECHNICAL FIELD

The present invention relates to methods of screening, drug discovery and high-throughput screening. Particularly, the present invention relates to a method of screening active agents of cell-surface receptors and ion channels, in particular G protein-coupled receptors (GPCRs), to cells expressing a recombinant and/or a chimeric receptor, to nucleotide sequences, amino acid sequences and cells useful in the methods of the invention.

### PRIOR ART AND THE PROBLEM UNDERLYING THE INVENTION

GPCRs comprise one of the largest protein superfamilies and the most diverse form of transmembrane signaling proteins. It has been estimated that 1% of the mammalian genome encodes GPCRs and about 450 of 950 predicted human GPCRs are expected to be receptors for endogenous ligands. The ligands, which bind to these receptors, activate them and trigger their respective signal transduction processes, include light-sensitive compounds, odors, pheromones, hormones (endocrine, exocrine and paracrine) and neurotransmitters. Upon binding of a ligand, GPCRs activate different pathways inside the cell, leading, for instance, to changes in intracellular levels of cyclic adenosine monophosphate (cAMP) and/or phosphatidylinositol (PI).

Receptors belonging to this family are broadly distributed in the body and are involved in a wide variety of physiological processes. In the nervous system, for example, they are involved in key physiological processes including cognition, mood, appetite, pain, synaptic transmission, vision, taste and smelling of odors. Among their other functions, they also have a pivotal role in immune response regulation and inflammation, as well as in metabolic processes. They have been implicated in a plethora of diseases, which makes them most important drug targets today. It has been estimated that almost 40% of all current therapeutic agents, mostly identified in ligand-binding assays, act upon GPCRs in a competitive manner to the natural ligand.

Possible therapeutic agents acting upon GPCRs can be orthosteric ligands, binding to the same site as natural ligands of GPCRs, which can be either agonists, that activate GPCRs in a manner similar to a natural ligand, or antagonists, that do not activate the receptor nor trigger the signaling cascade, but act in a competitive manner to agonists or natural ligands, by preventing their binding to the same orthosteric site, thus indirectly inhibiting their effects. A third kind of potential therapeutics binding to the orthosteric binding site are inverse agonists that stabilize the receptor in a more inactive state than it is in its resting state, when no ligand is bound. Inverse agonists are in competition for the receptor's orthosteric binding site with both natural ligands (or agonists) and antagonists.

Alternatively, other possible therapeutic agents can be, for example, allosteric modulators, which are substances that bind to receptors at a site termed allosteric binding site (or alternative binding site), which is topographically distinct from the above-mentioned orthosteric binding site that binds orthosteric ligands. The binding of an allosteric modulator to its binding site generally induces a conformational change of the receptor. The transmission of this conformational change from the allosteric to the orthosteric binding site and/or directly to effector-coupling sites is believed to enable allosteric ligands to modulate or fine-tune receptor activity. Depending on the nature of fine-tuning of receptor activity by allosteric modulators, they can either be positive, if they enhance the activity of orthosteric agonists, or negative allosteric modulators, if they inhibit it.

Methods of screening for orthosteric candidate ligands, agonists and antagonists, can be binding assays, which use, for example, a radioactively labeled known orthosteric ligand to label the receptor. In the screening process, a candidate ligand is added in the same sample as the known labeled orthosteric ligand and, if the candidate ligand is a positive hit, it will be in competition for the binding site with the known labeled orthosteric ligand and thus change (typically diminish) the signal from such a sample, when compared to a control sample with the known labeled orthosteric ligand alone. The information about the candidate compounds obtained from such a screen contains only their affinity for the target receptor, while the cellular context, i.e. functionality in terms of the cell signaling pathway related to the function of the target, is lost.

These above-described binding assays cannot be used for the screening for allosteric modulators, as the known labeled orthosteric ligand and allosteric modulators do not bind to the same binding site. The assays used for the screening for allosteric modulators will thus typically be assays measuring the function of the receptor i.e. functional assays. Of note, functional assays can also be used for the screening for orthosteric ligands.

Most of the functional assays used today are end-point assays, which monitor the accumulation of second messenger molecules following receptor activation. One such assay is, for example, the measurement of radioactively labeled GTPγ[³⁵S] or, more precisely, its accumulation over time (the samples are typically read after 30-90 minute incubation of the candidate compound with membranes expressing the receptor target), that is used to quantify the activation of a Gα-protein, the first step in the activation cascade of a GPCR.

Although this assay was widely used in HTS in the past, one of its drawbacks is that the assay is long, while a cellular response to the candidate receptor may be short and/or transient and, thus, more difficult to detect with such an end-point assay. The time delay from the incubation of the candidate compound and the receptor to the actual measurement can, in this case, result in false negative results i.e. missing potential hits in the screening.

Another disadvantage of this end-point assay is that it is mostly run in 96-well plates, as its sensitivity significantly decreases when further miniaturized, which makes it unsuitable in modern HTS practice. Moreover, this particular experimental system uses membrane preparations expressing the GPCR of interest and not live cells, as the radioactive tracer does not penetrate the cell-membrane. This also makes the assay less suitable for HTS, as preparation of the amount of membranes needed for a HTS campaign is long, cumbersome and costly. An additional handicap is that it is an end-point or “offline” assay, so it gives no opportunity to the experimentator for live or “online” monitoring of the events that happen in each sample. Finally, a crucial shortcoming of functional assays in screening for both orthosteric ligands and allosteric modulators is the ambiguity about the specificity of a positive hit (or detection of false positive “hits”). For example, in the case of the above-described GTPγ[³⁵S] assay system, it cannot be excluded that a positive “hit” detected with this assay actually acts on another unknown endogenously expressed GPCR that also activates a Gα-protein that generates the signal. This phenomenon is known in pharmacology as “receptor crosstalk” or “receptor-receptor crosstalk”.

Receptor crosstalk is often described as activation of one receptor by its ligand that affects the responses of other receptors. This occurs mainly as interferences in shared biochemical pathways downstream of the primary ligand-receptor interaction event. For example, activation of certain receptors affects signaling by other receptors that share the same pool of G-proteins, target signal-generating enzymes (such as, for example, adenylate cyclase) or other downstream effector proteins. There could also be more complex scenarios of receptor crosstalk, such as receptors belonging to one receptor family that can affect signaling by members of another receptor family via pathways interacting at mutual cellular regulatory proteins. The complexity of cellular signaling is underscored by the fact that hundreds of interacting receptors, G-proteins and effector proteins are typically expressed in a single cell. This is principally why end-point functional assays, which measure accumulation of second messenger molecules, generally share a problem of specificity of positive hits.

As at least 7 different Gα-protein subunits (Gαs, Gαi₁₋₃, Gαq, Gα₁₁ and Gα₁₃) are commonly co-expressed in individual cells. One strategy employed for examining specific GPCR-Gα interactions, mostly in the GTPγ[³⁵S] assay, is the use of fusion proteins (see review by Milligan et al., Current Opinion in Pharmacology 2007, No. 7, pp. 521). GPCR-Gα fusions generally link the C-terminus of a GPCR of interest to the N-terminus of the Gα-subunit which ensures physical proximity of the GPCR of interest and its Gα subunit and their 1:1 stoichiometry. Moreover, the use of such fusions has been reported in the past in a 96-well plate format GTPγ[³⁵S] assay to examine orphan receptors and/or receptors with very low signal/noise ratio, as the physical proximity of the GPCR target and the Gα subunit is likely to increase the signal/noise ratio of the assay. While the problem of low signal/noise ratio and the problem of specificity (i.e. detection of false positive hits) may well be improved by the use of such fusion proteins, the GTPγ[³⁵S] assay still remains unsuitable for modern HTS practice due to other reasons stated above.

Other examples of end-point functional assays that are widely used to monitor GPCR activation and that suffer from the problem of generating false negative (i.e. missing positive hits) and false positive (i.e. “picking up” hits non-specific for the target receptor) results mainly monitor accumulation of second messenger molecules that follow receptor activation. These are, for example, immunoassays used for quantification of cAMP or inositol-triphosphate (IP3) production or assays measuring reporter gene expression, or the deactivation of GPCR signaling by monitoring internalization of receptor upon binding to a protein called β-arrestin. All these mentioned experimental systems utilize long and non-physiological incubations of the candidate compound with cells expressing the target receptor, from minimally 20 minutes in case of, for instance, cAMP immunoassays to as long as 24 hours in several reporter gene assays. In that given time, candidate compounds often exert non-specific effects and are “picked up” as false positive hits. One additional problem of long incubation times of the candidate compound with a cellular system expressing a target receptor is receptor desensitization and down-regulation, which results in a loss of overall signal and in false negative results.

There are examples of functional assays that are dynamic and allow “online” monitoring and short measurements, but they suffer from specificity problems due to the measurement of a reporter that is distant from the activation event of the receptor. One such example is the measurement of the calcium flux into the cytoplasm of the cell upon GPCR activation. The signaling cascade steps are the following: translocation of the G-Protein subunit Gα to phospholipase C(PLC), conversion of phosphatidylinositol bisphosphate (PIP2) into diacylglycerol (DAG) and IP3 by PLC, binding of free cytosolic IP3 to the IP3-gated Calcium-channels in the endoplasmic reticulum membrane and Ca²⁺-flux into the cytosol. Measurement of intracellular Ca²⁺-flux can be done in either a fluorescent manner, which requires co-expression of the target GPCR and a chimeric or promiscuous G-protein that couples the signaling cascade to the PLC pathway in case the target GPCR does not naturally couple to it, or a luminescent one, which additionally requires co-expression of a luminescent calcium sensor such as, for example, the Aequorin protein. The incubation of a candidate compound and the above-mentioned cellular system is generally short and monitoring of events in all samples is measured “online”, so the danger of missing positive hits may indeed be lower than in end-point assays. However, despite the dynamicity of this assay, its drawbacks remain specificity and the fact that GPCR targets that do not normally couple to the PLC pathway need to be artificially coupled to it, which can result in even more false positive hits.

More recently, other dynamic functional assays have been developed, some which are using reporter systems based on resonance energy transfer, such as Förster resonance energy transfer (FRET) and bioluminescence resonance energy transfer (BRET). For example, Jiang et al. (Journal of Biological Chemistry, 282, no. 14, 2007) report a cAMP BRET sensor that is able to characterize regulation of cAMP by a novel sphingosine-1-phosphate/G₁₃ pathway. Their sensor “CAMYEL” comprises an EPAC cAMP binding domain fused between a yellow fluorescent protein (YFP) and a luciferase (RLuc). Resonant energy transfer is instant, which is why systems such as “CAMYEL” could in theory monitor dynamic changes upon the addition of candidate ligands.

In practice, however, while FRET and BRET systems described in the prior art provide valid results on laboratory scale, their application in industrial HTS procedures has proven to be difficult. In FRET, the signal is generated by light excitation of the fluorescent donor molecules. This can result in a range of problems including cell damage, photobleaching, low signal-to-noise ratios due to reflections form the assay plate, the intrinsic cellular autofluorescence, and in particular direct excitation of the acceptor molecule. The use of a bioluminescent donor molecule in BRET overcomes those problems and is generally regarded as a method of choice as it generated less background signal and results in a higher signal-to-noise ratio compared to FRET. However, only low light intensities are emitted by the luminescent and fluorescent proteins used in BRET sensors, which are only detectable by sophisticated detection equipment that is able to capture individual photons. Typically, photomultiplier tubes (PMTs) are employed. However, HTS based on an instrument using PMTs is slow, because only one or a few samples can be analyzed at the same time. When microtiter plates comprising 96 wells or more are analyzed, the time delay from incubation to actual measurement of a given sample may be such that the cellular response to a candidate compound of a library is not detected, because the cellular response at the potentially short moment when it is detectable by the reporting system may not yet have started or may be already over (problem of false negative results in HTS, see above for examples of GTPγ[³⁵S] and second messenger immunoassays).

In summary, even if the currently described molecular BRET tools inherently possess dynamic properties, current BRET reading set-ups, i.e. measurement with devices that use PMTs, do not permit their use in a dynamic fashion in an HTS format. They cannot be used for simultaneous “online” monitoring of all events in a screening plate because rapid repeated measurements of all samples are not possible. This however is critical for reliable finding of positive hits in HTS of allosteric modulators.

An additional drawback of the above-mentioned “CAMYEL” BRET cAMP biosensor is the possibility of non-specificity of positive hits, which refers to the problematic of receptor crosstalk discussed above. For example, in the case of a cAMP biosensor, it cannot be excluded that a positive “hit” detected in the assay actually acts directly on adenylate cyclase instead of activating the GPCR or on another unknown endogenously expressed receptor that also activates adenylate cyclase.

Even more importantly, the use of a cAMP sensitive sensor as “CAMYEL” is limited to the screening of candidate compounds of GPCR targets that signal through the cAMP signal transduction pathway. Candidate compounds of GPCRs that signal exclusively through the PLC pathway, or possibly yet unknown signal pathways, cannot be found using this prior art biosensor.

In summary, it is an objective of the present invention to provide a method of screening compounds that are capable of affecting a GPCR mediated cellular response, wherein said method is proximal to the activation step of the target receptor and thus specific for the target, able to measure rapid changes in all wells of a screening plate simultaneously (possessing dynamic or “online” properties), cell-based and sufficiently sensitive so as to detect low affinity binding active agents including allosteric modulators.

In other words, it would be advantageous to have a system allowing for observing cellular responses to allosteric and orthosteric effectors at a high temporal resolution, which should be capable to detect an early event in the signal transduction process following receptor binding and activation by an active agent.

More generally, it is an objective of the present invention to provide a method of screening candidate active principles at a high throughput, preferably entire libraries of potentially active compounds. It is an objective to provide HTS that is fast and specific for the target, but, at the same time, sensitive enough and adapted to detect subtle cellular responses and/or cellular responses of short or delayed duration, for example.

The present invention addresses the problems above and provides methods for achieving the objectives and purposes described above. The problems, objectives and goals described above are part of the present invention.

### SUMMARY OF THE INVENTION

Remarkably, the present inventors were able to provide new polypeptides, cells containing the polypeptide and methods of screening candidate active principles, which solve the above depicted problems. In particular, the inventors surprisingly disclose chimeric polypeptide receptors in combination with chimeric polypeptides involved in very early stages of receptor activation and signal transduction.

Surprisingly, said chimeric receptors and/or polypeptides can be used in novel HTS assays, which can monitor fast kinetic events following receptor activation in real-time before and after the addition of the candidate agent using BRET as a dynamic and “online” readout. The molecular tools described herein ensure detection of a natural response of a target receptor that is proximal to it (i.e. natural G-protein coupling), discovery of difficult-to-detect candidate compounds (e.g. with fast and/or transient responses) and elimination of artifacts originating from downstream receptor crosstalk.

In a first aspect, the present invention provides a method of screening active agents.

In a second aspect, the present invention provides a method of screening, the method comprising the steps of:
exposing a candidate active agent to cells containing at least a chimeric polypeptide receptor as disclosed and/or defined herein and/or expressing at least one nucleotide sequence encoding at least one polypeptide of the invention;
conducting measurements of light emitted from the cells; and,
determining, from said light measurements, if said candidate active agent is an active agent.

In a third aspect, the present invention provides a method of screening active agents, said method comprising the steps:
exposing a sample of cells to a candidate agent to be screened;
measuring, in an automated manner, the presence or absence of a reporting signal from a plurality of samples provided in wells of a microtiter plate; and
determining, from the reporting signal as measured, whether or not said candidate agent is considered an active agent and/or an active agent of interest.

In a fourth aspect, the present invention provides a method of screening, said method comprising the steps:
exposing a sample of cells to a candidate agent;
conducting, in an automated manner, a series of repeated measurements of light emitted from the sample, wherein said measurements are repeated within determined time intervals of 0.5 seconds to 10 minutes over a total period of up to 30 minutes;
wherein said sample of cells is provided in a well of a microtiter plate comprising a plurality of further wells with further samples, and wherein a plurality of different candidate agents and/or candidate agents at different concentrations are comprised in an individual microtiter plate;
wherein, in each of the repeated measurements light emission of all samples in the wells of an entire microtiter plate is measured at the determined time intervals simultaneously;
wherein said candidate agent is considered an active agent, if it has a detectable effect on the quantity of light of a specific wavelength or wavelength range emitted from and/or reflected by the cells of said sample.

In a fifth aspect, the present invention provides a method of screening for agents exhibiting an activity on and/or binding to a cell-surface G protein-coupled receptor (GPCR), said method comprising the steps:
exposing a sample of cells to a candidate agent;
wherein said cells express at least a recombinant nucleotide sequence encoding a chimeric protein (a) comprising a GPCR fused, in any order, to a Gα subunit and also to one selected from a luminescent protein and a fluorescent protein, and a recombinant nucleotide sequence encoding a chimeric protein (b) comprising a Gγ and/or a Gβ subunit fused to one selected from a luminescent protein and a fluorescent protein;
with the provisos that if (a) comprises a luminescent protein, the Gγ and/or the Gβ subunit of (b) comprises a fluorescent protein, and if the Gγ and/or the Gβ subunit of (b) comprises a luminescent protein, (a) comprises a fluorescent protein;
conducting measurements of light emitted from the sample,
   wherein said candidate agent is considered an active agent of a GPCR, if it has a detectable effect on the quantity of light units emitted and/or reflected by said protein (a) and/or said protein (b) compared to a sample devoid of said candidate agent.

In a sixth aspect, the present invention provides a method of high-throughput screening for compounds that are capable of affecting the activity of a G Protein-Coupled Receptor (GPCR), said method comprising the steps:
exposing a sample of cells to a candidate agent;
conducting, in an automated manner, measurements of light emitted from the sample;
wherein said cells express at least a recombinant nucleotide sequence encoding a chimeric protein (a) comprising an amino acid sequence of a GPCR fused to, in any order, the amino acid sequence of a Gα subunit and also to an amino acid sequence of one selected from a luminescent protein and a fluorescent protein, said cells also expressing a recombinant nucleotide sequence encoding a chimeric protein (b) comprising a Gγ and/or a Gβ subunit fused to one selected from a luminescent protein and a fluorescent protein;
with the provisos that if (a) comprises a luminescent protein, the chimeric protein (b) comprises a fluorescent protein, and if chimeric protein (b) comprises a luminescent protein, (a) comprises a fluorescent protein, and that the luminescent protein or fluorescent protein of chimeric protein (a) emits light of a different wavelength than the luminescent protein or fluorescent protein of the chimeric protein (b);
wherein said protein (a) and said protein (b) can exist in said cells in a first state, where there is no or little energy transfer, and a second state, where there is a substantial energy transfer from the luminescent protein or fluorescent protein of chimeric protein (a) to the luminescent protein or fluorescent protein of chimeric (b), or vice versa, wherein said energy transfer affects the quantity of measurable light emitted by said fluorescent protein(s) and/or said luminescent protein;
wherein an activation of said GPCR by said candidate agent determines if (a) and (b) are in said first or second state and therefore the quantities of light emitted from (a) and/or (b)

In a seventh aspect, the present invention provides a method of HTS for agents exhibiting an activity on and/or binding to a G protein-coupled receptor (GPCR), said method comprising the steps:
exposing a sample of cells to a candidate agent;
conducting, in an automated manner, a series of repeated measurements of light emitted from the sample, wherein said measurements are repeated within determined time intervals of 0.4 seconds to 10 minutes over a total period of up to 30 minutes;
wherein said cells express at least a recombinant nucleotide sequence encoding a chimeric protein (a) comprising an amino acid sequence of the GPCR fused to the amino acid sequence of a Gα subunit and also fused to an amino acid sequence of one selected from a luminescent protein and a fluorescent protein, and a recombinant nucleotide sequence encoding a chimeric protein (b) comprising a Gγ and/or a Gβ subunit fused to one selected from a luminescent protein and a fluorescent protein;
with the provisos that if (a) comprises a luminescent protein, the chimeric protein (b) comprises a fluorescent protein, and if chimeric protein (b) comprises a luminescent protein, (a) comprises a fluorescent protein, and that the luminescent protein or fluorescent protein of chimeric protein (a) emits light of a different wavelength than the luminescent protein or fluorescent protein of the chimeric protein (b);
wherein said protein (a) and said protein (b) can exist in said cells in a first state, where there is no or little energy transfer, and a second state, where there is a substantial energy transfer from the luminescent protein or fluorescent protein of chimeric protein (a) to the luminescent protein or fluorescent protein of chimeric (b), or vice versa, wherein said energy transfer affects the quantity of measurable light emitted by and/or reflected from said fluorescent protein(s) and/or said luminescent protein;
wherein an activation of said GPCR by said candidate agent determines if (a) and (b) are in said first or second state and therefore the quantities of light emitted from (a) and/or (b);
wherein said sample of cells is provided in a well of a microtiter plate comprising a plurality of further wells with further samples, and wherein a plurality of different candidate agents and/or candidate agents at different concentrations are comprised in a individual microtiter plate;
wherein, in each of the repeated measurements light emission of all samples in the wells of an entire microtiter plate is measured at the determined time intervals simultaneously;
wherein said candidate agent is considered an active agent, if it has a detectable effect on the quantity of light units emitted and/or reflected by said protein (a) and/or said protein (b) compared to a sample devoid of said candidate agent.

In an aspect, the method of the invention provides a method of high-throughput screening for agents, which are capable of affecting the activity of a G Protein-Coupled Receptor (GPCR), said method comprising the steps: exposing a sample of cells to a candidate agent; conducting, in an automated manner, measurements of light emitted from the sample; wherein said cells express at least a recombinant nucleotide sequence encoding a chimeric protein (a) comprising an amino acid sequence of a GPCR fused to the amino acid sequence of a Gα subunit, wherein, in said cells, said chimeric protein (a) is either further fused to a luminescent protein or is connected to a fluorescent entity, said cells also expressing a recombinant nucleotide sequence encoding a protein (b) comprising a Gγ and/or a Gβ subunit, wherein, in said cells, said protein (b) is either is connected to a fluorescent entity or further fused to a luminescent protein; with the provisos that if (a) comprises a luminescent protein, the protein (b) comprises a fluorescent entity, and if protein (b) comprises a luminescent protein, (a) comprises a fluorescent entity, and that the luminescent protein or fluorescent entity of chimeric protein (a) emits light of a different wavelength than the luminescent protein or fluorescent entity of the protein (b); wherein said protein (a) and said protein (b) can exist in said cells in a first state, where there is no or little energy transfer, and a second state, where there is a substantial energy transfer from the luminescent protein or fluorescent entity of chimeric protein (a) to the luminescent protein or fluorescent entity of protein (b), or vice versa, wherein said energy transfer affects the quantity of measurable light emitted by said fluorescent protein(s) and/or said luminescent protein; wherein an activation of said GPCR by said candidate agent determines if (a) and (b) are in said first or second state and therefore the quantities of light emitted from (a) and/or (b); wherein said candidate agent is considered an active agent if it has a detectable effect on the quantity of light units emitted by said protein (a) and/or said protein (b) compared to a sample devoid of said candidate agent.

In an aspect, the present invention provides a nucleotide sequence encoding a chimeric protein (a), said nucleotide sequence comprising a nucleotide sequence part encoding a GPCR, a nucleotide sequence part encoding a Gα subunit, and a nucleotide sequence part encoding one selected from a luminescent protein and a fluorescent protein. Preferably, said sequence parts are translationally fused to each other in the indicated order.

In a further aspect, the present invention provides a nucleic acid molecule comprising a nucleotide sequence encoding a chimeric protein (a) comprising a GPCR fused to a Gα subunit and also fused to one selected from a luciferase and a fluorescent protein.

In an aspect, the invention provides a nucleotide sequence encoding a chimeric protein (a), said nucleotide sequence comprising a nucleotide sequence part encoding a GPCR, a nucleotide sequence part encoding a Gα subunit, and a nucleotide sequence part encoding one selected from a luminescent protein and a fluorescent protein.

In another aspect, the present invention provides a chimeric protein (a) comprising a GPCR fused, in any order, to a Gα subunit and to one selected from a luciferase and a fluorescent protein.

In another aspect, the invention provides a chimeric protein (a) comprising a GPCR fused to a Gα subunit and either also fused to a luminescent protein or connected to a fluorescent entity.

In another aspect, the present invention provides chimeric proteins (b) as defined herein and nucleotide sequences encoding said protein (b).

In further aspects, the invention provides cells expressing one or more of the nucleotide sequences of the invention, cells containing one or more of the chimeric polypeptides disclosed herein and/or cells, in the plasma membrane of which is embedded a chimeric polypeptide (a) as defined herein.

The present invention also encompasses the use of chimeric proteins (a) and/or (b) in screening methods, in particular in HTS.

Further aspects and preferred embodiments of the invention are defined in the description below and in the appended claims.

### BRIEF DESCRIPTION OF THE DRAWINGS

FIG. 1 schematically represents the fusion constructs of preferred embodiments of the present invention.

FIGS. 2 A-2 C schematically show the preparation of vectors suitable for the expression of the fusion polypeptides RLG, RGL, Gαs¹⁻⁷¹-RLuc-Gαs⁸²⁻³⁹⁴ (GLG1), Gαo¹⁻⁹¹-RLuc-Gαo⁹²⁻³⁵⁴ (GLG2) and Gq¹⁻¹²⁴-RLuc-Gq¹²⁵⁻³⁵⁹ (GLG3) respectively, according to different embodiments of the present invention.

FIG. 3 A shows a concentration-response curve obtained with the method according to an embodiment of the present invention. Cells co-expressing a construct comprising a gastric inhibitory polypeptide (GIP) receptor (GIPR) fused to a Gαs protein and further fused to a luciferase (fusion construct RGL in FIG. 1) and the Gγ9 protein fused to a fluorescent protein at its C-terminus (GT-FP) were exposed to increasing concentrations of GIP, a reported natural ligand (orthosteric agonist) of the GIP receptor (GIPR) (full squares). Upon receptor activation, a change of the BRET (Bioluminescence Resonance Energy Transfer) ratio is detected, indicating a dissociation of the Gγ9 protein from the chimeric receptor protein. The maximal GIP response corresponds to the highest value of the ratio of blue light to yellow light, or, in other words, to the lowest level of BRET. The graph also shows the basal value, obtained upon addition of buffer (empty square). The data on graph represent mean values±SEM of duplicates and they are normalized to the maximal effect of the orthosteric agonist after the subtraction of the basal values.

FIG. 3 B shows a concentration-response curve as FIG. 3 A, with the difference that the GIPR construct used in this example is first fused to a luciferase and the latter is further fused to a Gαs protein (fusion construct RLG in FIG. 1). As in FIG. 3A, cells are also co-expressing the Gγ9 protein fused to a fluorescent protein at its C-terminus (GT-FP). Cells were exposed to increasing concentrations, as indicated, of GIP (full squares). The graph also shows the basal value, obtained upon addition of buffer (empty square). The data on graph represent mean values±SEM of duplicates and they are normalized to the maximal effect of the orthosteric agonist after the subtraction of the basal values.

FIG. 4 shows a concentration-response curve as FIGS. 3A and B, but with a chimeric acetylcholine muscarinic₄ receptor (M₄R) that is first fused to a part of the Gαo aminoacids 1-91, followed by the sequence of the luciferase and finally by the Gαo aminoacids 92-354 (fusion construct RGLG in FIG. 1, using GLG2 from Table 6).

FIGS. 5 A to C show concentration response curves of cells co-expressing different embodiments, respectively of the chimeric polypeptides of the invention. In all three figures, values obtained with different concentrations of a natural or reported agonist are shown, as well as values obtained with the addition of EC₈₀ concentration of the natural or reported agonist alone (empty circles) and in combination with different concentrations of a selected active principle (antagonists), and basal values obtained by addition buffer alone (empty square), as indicated in the figures. The data on graph represent mean values±SEM of duplicates and they are normalized to the maximal effect of the orthosteric agonist after the subtraction of the basal values.

FIG. 5 A shows concentration-response curves of cells co-expressing a construct comprising a glucagon-like peptide-1 receptor (GLP-1R) fused to a luciferase and further fused to a Gαs protein (fusion construct RLG in FIG. 1) and the Gγ9 protein fused to a fluorescent protein at its N-terminus (FP-Gγ9) in accordance with the present invention. The cells were exposed to different compounds in increasing concentrations: GLP-1(7-36), a reported natural ligand (orthosteric agonist) of the GLP-1R (full circles); Exendin(9-39), a reported competitive antagonist of GLP-1R administrated in the presence of the EC₈₀ concentration of GLP-1(7-36) (full squares). Additionally, in this figure Novo Nordisk compound 2, a reported allosteric agonist, is also shown when it was administered alone (Novo2, empty diamonds).

FIG. 5 B shows a concentration-response curve of cells co-expressing a construct comprising a gastric inhibitory polypeptide (GIP) receptor (GIPR) fused to a Gαs protein and further fused to a luciferase (fusion construct RGL in FIG. 1) and the Gγ9 protein fused to a fluorescent protein at its N-terminus (FP-Gγ9) in accordance with the present invention. Cells were exposed to different compounds in increasing concentrations: GIP, a reported natural ligand (orthosteric agonist) of the GIP receptor (GIPR) (full squares), [Pro³]-GIP, a competitive antagonist of GIP, in presence of the EC₈₀ value of GIP (full inverted triangles), GIP (6-30), a competitive antagonist of GIP, in presence of the EC₈₀ value of GIP (full circles).

FIG. 5 C shows concentration-response curves in which a chimeric adenosine_{2A} receptor (A_{ZA}R), fused to a Gαs protein and further fused to the luciferase (fusion construct RGL in FIG. 1), was used co-expressed with FP-Gγ9 as above. 5′-N-ethylcarboxamidoadenosine (NECA, full circles) was used as agonist. In different wells, cells were exposed to increasing concentrations of SCH442416, a reported A_{2A}R-selective competitive antagonist, in presence of the EC₈₀ value of NECA (full squares).

FIG. 6 shows the specificity of the response of the chimeric GIPR (construct and cell-line from FIG. 5B). Specifically, it shows activation of the chimeric GIPR by increasing concentrations of its natural ligand GIP, and in contrast, lack of activation by increasing concentrations of ligands of other receptors potentially endogenously expressed in the HEK cellular background (as shown in FIG. 25 for example). These ligands are the following: NECA (full diamonds), Corticotropin releasing factor (CRF, empty circles), calcitonine (full triangles), carbachol (empty triangles), isoproterenol (full squares) and uridine-5′-triphosphate (UTP, empty diamonds).

FIG. 7 A shows dynamic traces of the response of the chimeric A_{2A}R (construct RGL, FIG. 1) to various concentrations of agonist NECA over time (full circles on FIG. 5C). Exposure time for both, blue and yellow, channels was 2.5 seconds. The two arrows indicate the addition of buffer (left arrow, 1^{st} addition) and the addition of NECA (right arrow, 2^{nd} addition) to the cells. The 1^{st} addition (buffer) took place 80 seconds after the beginning of the read, and the 2^{nd} addition (NECA) after 300 seconds. The traces are means of smoothened duplicates (smoothen function, GraphPad Prism® software version 5, 4 neighbours, 0 order) for the purposes of this graph only. A mean buffer value was subtracted from all traces to set the starting ratio to 0.

FIG. 7 B shows dynamic traces of the response of the chimeric GIPR (construct RLG, FIG. 1) to various concentrations of GIP over time (full squares on FIG. 5B). Exposure time for both, blue and yellow, channels was 2 seconds. GIP was added to the cells after 100 seconds (indicated by the arrow). The traces are means of smoothened duplicates (smoothen function, GraphPad Prism® software version 5, 4 neighbours, 0 order) for the purposes of this graph only. A mean buffer value was subtracted from all traces to set the starting ratio to 0.

FIG. 7 C shows dynamic traces of the response of the chimeric muscarinic acetylcholine receptor (M₄R) (construct RGLG, FIG. 1, using GLG2 from Table 6) to various concentrations of acetylcholine (ACh) over time (full squares on FIG. 4). Exposure time for both, blue and yellow, channels was 2 seconds. ACh was added to the cells after 100 seconds (indicated by the arrow). The traces represent mean values of non-smoothened quadruplicates. A mean buffer value was subtracted from all traces to set the starting ratio to 0.

FIG. 8 shows basal, EC₂₀ and EC₁₀₀ values of GIP on the chimeric BRET system of the GIPR, described in FIG. 5B, that were used for the calculation of their respective Z′-factor values (see Methodology, Results and Z′-Factor of the Screening Experiments section below). A mean of all basal values was subtracted from all traces to set the starting ratio to 0.

FIGS. 9-26 show screening methods according to different embodiments of the present invention in which different GPCRs with reported and other modulators were tested. In FIGS. 21-24 embodiments of the method of the invention are compared with state of the art calcium assays. Generally, cells were exposed to different (increasing) concentrations of test compounds as indicated in the figures. Most graphs show the basal value (except FIGS. 10B, 10C, 11B), obtained upon addition of buffer (symbol as indicated in the respective legend). In several figures, EC₂₀ and/or EC₈₀ values of the orthosteric agonist alone are shown, as indicated in the legend of the respective figure (FIGS. 9, 10A, 11A, 12, 15, 16, 17, 18). The data on graphs represent mean values±SEM of duplicates and they are normalized to the maximal effect of the orthosteric agonist after the subtraction of the basal values.

FIG. 9 shows a concentration-response curve obtained with the method of an embodiment of the invention. Cells were co-expressing a construct comprising an adenosine_{2A} receptor (A_{2A}R), fused to a Gαs protein and further fused to a bioluminescent protein (fusion construct RGL in FIG. 1) and the FP-Gγ9 construct (as above). Cells were exposed to different compounds in increasing concentrations: Adenosine, a reported natural ligand (orthosteric agonist) of the A_{2A}R (full squares), 2-chloro-Adenosine, a specific agonist of the A_{2A}R (full triangles), MRS1754, a reported A_{2A}R competitive antagonist, in presence of the EC₈₀ value of Adenosine (full diamonds), SCH58261, a reported A_{2A}R competitive antagonist, in presence of the EC₈₀ value of Adenosine (full circles), ZM241385, a reported A_{2A}R competitive antagonist, in presence of the EC₈₀ value of Adenosine (empty circles).

FIG. 10 A shows a concentration-response curve obtained with the method of an embodiment of the invention. Cells used for the assay co-express a construct comprising a human muscarinic 4 receptor (M4R) fused to a split Gαo protein harboring a luminescent protein (fusion construct RGLG-2 in FIG. 2C, Table 6) and the FP-Gγ9 construct as above. Cells were exposed to different compounds in increasing concentrations: Acetylcholine (Ach), a reported natural ligand (orthosteric agonist of M4R) (full squares), LY2033298, a positive allosteric modulator (PAM)/agonist of M2R and M4R (empty inverted triangles), and VU152100, PAM of M4R in presence of the EC₂₀ value of Achetylcholine (empty triangles), Tropicamide, antagonist of M2R and M4R (empty squares), and WIN51708, a negative allosteric modulator (NAM) of M4R (empty circles), in presence of the EC₈₀ value of Achetylcholine.

FIG. 10 B demonstrates the involvement of the Gα protein of the construct of the invention in BRET-signalling. The same cells as described for FIG. 10 A were used. Cells were incubated over night in culture medium containing 100 ng/ml of Gαi/o-inhibiting Bordetella pertussis toxin (PTX) (empty circles) or vehicle (full squares) and were exposed to Acetylcholine (Ach) in increasing concentration. PTX effectively blocks the BRET signal changes compared to the non-PTX treated condition by promoting the ADP-ribosylation of the C-terminal Cysteine³⁵¹ of Gαo, indicating that the PTX-sensitive Gαi/o are involved in the signalling. The decrease of the signal below baseline in the PTX-treated condition confirms a constitutive activity that has been described for the M4R.

FIG. 10 C shows the same experimental conditions as FIG. 10 B using a PTX-insensitive mutant (C351S) of Gαo in the RGLG-2 fusion construct. The Gαo-inhibiting effects of PTX are rescued by the C351S substitution, demonstrating that the G protein used in the fusion construct, and not the ones expressed endogenously by the cells, is the one actively participating in the BRET signalling.

FIG. 11A shows a concentration-response curve obtained with the method of an embodiment of the invention. Cells co-express a construct comprising a human M1 receptor (M1R) fused to a split Gαq protein harboring a luminescent protein (construct RGLG-3, FIG. 2C, Table 6) and the FP-Gγ9 construct (as above). Cells were exposed to different compounds in increasing concentrations: Acetylcholine (Ach), a reported natural ligand (orthosteric agonist of M1R) (full squares), BQCA, a positive allosteric modulator (PAM) of M1R, in presence of the EC₂₀ value of Ach (empty triangles), Scopolamine (empty squares) and Pirenzepine (empty circles) (antagonists of M1R) in presence of the EC₈₀ value of Achetylcholine.

FIG. 11B corresponds to FIG. 10 B, but using the cells as described in FIG. 11A (M1R-GLG-3, FP-Gγ9). Overnight incubation with PTX (empty squares) or vehicle (full squares) shows no effect on the BRET signal changes obtained in response to Ach, demonstrating that PTX selectively blocks Gαi/o, while not affecting Gαq signaling.

FIG. 12 shows a concentration-response curve obtained with the method of an embodiment of the invention. Cells co-express a construct comprising a human M2 receptor (M2R) fused to a split Gαo protein harboring a luminescent protein (fusion construct RLGL-2 in FIG. 2C, Table 6) and the FP-Gγ9 construct (as above). Cells were exposed to different compounds in increasing concentrations: Acetylcholine (Ach), a reported natural ligand (orthosteric agonist of M2R) (full squares), LY2033298 (allosteric agonist/PAM of M4R and M2R) (empty inverted triangles) and VU152100 (PAM of M4R) (empty triangles) in presence of the EC₂₀ value of Ach, Tropicamide (antagonist of M2R and M4R) (empty squares) and WIN51708 (empty circles) (NAM of M4R) in presence of the EC₈₀ value of Ach.

FIG. 13 shows a concentration-response curve obtained with the method of an embodiment of the invention. Cells were co-expressing a construct comprising a growth hormone secretagogues (GHS) receptor (GHSR) fused to a luminescent protein and further fused to a Gαq protein (fusion construct RLG in FIG. 1) and the FP-Gγ9 construct (as above). Cells were exposed to increasing concentrations of Ghrelin, a reported natural ligand (orthosteric agonist) of the GHS receptor (GHSR) (full squares).

FIG. 14 shows a concentration-response curve obtained with the method of an embodiment of the present invention. Cells were co-expressing a construct comprising a glucagon-like peptide-2 receptor (GLP-2R) fused to a luminescent protein and further fused to a Gαs protein (fusion construct RLG in FIG. 1) and the FP-Gγ9 construct (as above). Cells were exposed to increasing concentrations of GLP-2, a reported natural ligand (orthosteric agonist) of the GLP-2 receptor (GLP-2R) (full squares).

FIG. 15 shows a concentration-response curve obtained with the method of an embodiment of the present invention. Cells were co-expressing a construct comprising a glucagon receptor (GCGR) fused to a luminescent protein and further fused to a Gαs protein (fusion construct RLG in FIG. 1) and the FP-Gγ9 construct (as above). Cells were exposed to different compounds in increasing concentrations: Glucagon, a reported natural ligand (orthosteric agonist) of the glucagon receptor (GCGR) (full squares), 13-alanine GCGR antagonist, a competitive antagonist of Glucagon, in presence of the EC₈₀ value of Glucagon (full diamonds), GLP-1 (7-36) administered alone (full circles), GLP-2 administered alone (full triangles) and GIP administered alone (full inversed triangles).

FIG. 16 shows a concentration-response curve obtained with the method of an embodiment of the present invention. Cells were co-expressing a construct comprising a metabotropic glutamate receptor 2 (mGluR2), that is first fused to a split Gαo protein harboring a luminescent protein (fusion construct RGLG-2, FIG. 2C, Table 6) and the FP-Gγ9 construct (as above). Cells were exposed to different compounds in increasing concentrations: Glutamate, a reported natural ligand (orthosteric agonist) of the mGluR2 (full squares), LY379268, a specific agonist of the mGluR2 (full diamonds), ADX90206, a NAM in presence of the EC₈₀ value of Glutamate (full triangles), LY487379, a reported PAM in presence of the EC₂₀ value of Glutamate (full inverted triangles), ADX71151, a PAM in presence of the EC₂₀ value of Glutamate (full circles).

FIG. 17 shows a concentration-response curve obtained with a method according to an embodiment of the present invention. Cells were co-expressing a construct comprising a orexin 1 receptor (O×1R) fused to a luminescent protein and further fused to a Gαq protein (fusion construct RLG in FIG. 1) and the FP-Gγ9 construct (as above). Cells were exposed to different compounds in increasing concentrations: Orexin A, a reported natural ligand (orthosteric agonist) of the orexin 1 receptor (O×1R) (full squares), SB-408124, a competitive antagonist of 0x1R, in presence of the EC₈₀ value of Orexin A (full circles).

FIG. 18 shows a concentration-response curve obtained with a method according to an embodiment of the present invention. Cells were co-expressing a construct comprising a orexin 2 receptor (O×2R) fused to a luciferase and further fused to a Gαq protein (fusion construct RLG in FIG. 1) and the FP-Gγ9 construct (as above). Cells were exposed to different compounds in increasing concentrations: Orexin A, a reported natural ligand (orthosteric agonist) of the orexin 2 receptor (O×2R) (full squares), TCS OX2 29, a competitive antagonist of 0x2R, in presence of the EC₈₀ value of Orexin A (full circles).

FIG. 19 shows a concentration-response curve obtained with the method according to an embodiment of the invention. Cells were co-expressing the RGL construct comprising GIPR (FIG. 3A) and one of different constructs of various Gγ proteins (Gγ1, Gγ2, Gγ9) fused to a fluorescent protein at its N- or C-terminus (FP-Gγ, Gγ-FP, respectively), as indicated in the legend of the figure. Cells were exposed to increasing concentrations of GIP. The results show that the invention is workable with different Gγ proteins and in various orientations fused to the (fluorescent) reporter protein, as is also seen from Tables 7 and 8 of the examples.

FIG. 20 shows a concentration-response curve obtained with the according to an embodiment of the invention. Cells were co-expressing RGLG-2 construct comprising M₄R (FIG. 10 A) and different constructs of various Gγ proteins (Gγ2, Gγ7, Gγ8, Gγ9) fused to a fluorescent protein at its respective N-terminus (FP-Gγ). Cells were exposed to increasing concentrations of Ach (see also Tables 7 and 8).

FIG. 21 shows concentration-response curves of ADX95477, a PAM of GLP-1R, in presence of EC₂₀ value of GLP-1 in 2 different assays. In calcium assay, a calcium flux is measured in cells co-expressing recombinant GLP1-R and Gcc16 (full circles) and labelled with Fluo-4 (Molecular Probes). In the assay of an embodiment of the present invention, BRET was measured in cells co-expressing a chimeric GLP-1R RLG (construct and cell-line see description of FIG. 5A) and the FP-Gγ9 construct (full inverted triangles). It can be seen that ADX95477 is significantly detected at low concentrations with the assay of an embodiment of the present invention, whereas it is not detected by the calcium assay.

FIG. 22 shows affinity measurement for radioligand ^{125I}-GLP1 binding in presence of ADX95477 on membrane expressing GLP-1R (see FIG. 21). It can be seen that ADX95477 behaves like a PAM as it increases the affinity of GLP-1 receptor for its ligand GLP1. The effect is concentration dependent.

FIG. 23 shows concentration-response curves of ADX95615 in presence of EC₂₀ value of GLP-1 in 2 different assays. In the calcium assay, a calcium flux is measured in cells co-expressing GLP1-R and Gcc16 (full circles) and labelled with Fluo-4 (Molecular Probes). In the assay of an embodiment of the present invention, BRET is measured in the same GLP-1R-RLG and FP-Gγ9 co-expressing cells as described with respect to FIG. 21 above (full inverted triangles). ADX95615 was falsely determined to be a hit (“false-positive”) PAM by the calcium assay, whereas the assay in accordance with the invention did (correctly) not indicate this compound as being active on the GLP-1R receptor.

FIG. 24 shows affinity measurement for radioligand ^{125I}-GLP1 binding in presence of ADX95615 on membrane expressing GLP-1R. No change of affinity of the GLP-1R for its ligand GLP-1 was detected with increasing concentrations of ADX95615, suggesting this compound has no PAM activity.

FIG. 25 shows by applying increasing concentrations of various ligands, the activation, measured here by cAMP accumulation, of receptors endogenously expressed in non-transfected HEK cells. These ligands are the following: NECA (empty inverted triangles), Corticotropin releasing factor (CRF) (empty diamonds), calcitonin (full circles), isoproterenol (full triangles), UTP (full inverted triangles) and carbachol (full squares). An increase in cAMP demonstrates that these receptors are expressed endogenously in these host cells.

FIG. 26 shows the specificity of the response of the chimeric M₄R (construct and cell-line from FIG. 10A). Specifically, it shows activation of the chimeric M₄R by increasing concentrations of its natural ligand Ach (full circles), an allosteric agonist, LY2033298 (full squares) and an agonist, Carbachol (full triangles), and in contrast, lack of activation by increasing concentrations of ligands of other receptors endogenously expressed in host HEK cells as demonstrated in FIG. 25. These ligands are the following: isoproterenol (full inverted triangles), ATP (full diamonds), NECA (empty squares), Corticotropin releasing factor (CRF) (empty inverted triangles), and calcitonin (empty diamonds).

### DETAILED DESCRIPTION OF THE PREFERRED EMBODIMENTS

The present invention provides methods of screening compounds and/or compositions of matter exhibiting and/or exerting an activity, in particular a biological activity, on a receptor, in particular a G protein-coupled receptor (GPCR). Preferably, “activity” refers to receptor activating, inhibiting and/or modulating and/or cell signalling activity. Such compounds may be referred to as “active agents” or “active principles” in this specification. According to an embodiment, “compounds that are capable of affecting the activity of a G Protein-Coupled Receptor (GPCR)” comprise compounds that in some way can affect or modulate the conformational status of the GPCR and/or of the affinity of the GPCR to its respective Ga subunit. Furthermore, such compounds comprise compounds that can affect the affinity of the GPCR to other ligands, such as to one or more of the natural ligands, orthosteric and/or allosteric binding ligands. In this regard, a compound may affect the activity of the receptor only in presence of another ligand of the receptor.

Active agents or compounds, as understood in this specification, encompass agonists, inverse agonists, antagonists and modulators, for example. The agents may be binding to orthosteric and/or allosteric sites of a GPCR and/or the polypeptide of the invention. The terms agonists and antagonists encompass natural ligands—endogenous (ant)agonists—as well as exogenous (ant)agonists.

Modulators are generally compounds that act in a modulating manner mainly and/or only in conjunction with an agonist or antagonist, in particular with a natural ligand. Modulators may again be classified as “active modulators” on the one hand, which encompass and preferably consist of “inhibitors” and “activators”, and, on the other hand, “neutral modulators” of a receptor. “Neutral modulators” are chemical entities that bind to the target without direct activation, inhibition or modulation of its function, but they prevent the binding of the natural ligand and/or other modulators or bioactive principles that share the same binding site on the target receptor, and in that way indirectly affect its activity. Interestingly, the method of the present invention is suitable to screen also for compounds that act in a modulating manner mainly and/or only in conjunction with an agonist For example, an agent binding to or close to the orthosteric or allosteric site and preventing a natural ortho- or allosteric ligand, respectively, from binding to its site on the receptor is also an agent that affects the activity of the receptor for the purpose of this specification.

According to an embodiment, the method of the present invention is suitable to screen for PAMs (positive allosteric modulators) and NAMs (negative allosteric modulators), which increase or inhibit the activity of a ligand, such as a natural orthosteric ligand, for example.

According to an embodiment, the invention provides a method for screening active agents, in particular of a GPCR. Preferably, the GPCR is a cell-surface GPCR. According to an embodiment, the invention provides a method for screening also allosterically binding active agents of a GPCR, such as allosteric modulators.

For the purpose of the present specification, the expression “candidate”, such as in the expressions “candidate agent”, for example, is a substance of matter, in particular a molecule, that is screened in the screening methods of the invention, and for which the activity on a target receptor is assessed in the course of the screening process. Accordingly, a “candidate agent” may actually not be, and will in most cases not be, an active agent of the target receptor. However, the screening process has the purpose of finding out if a candidate agent has an activity. A “candidate agent” may also be any composition of matter, for example a composition comprising several chemical molecules. For example, a biological extract, such as a plant extract is also encompassed by the term “candidate agent”.

According to a preferred embodiment, the present invention relates to high-throughput screening (HTS). The term “high-throughput screening” refers to the screening of a plurality of samples in an automated process. Automation may refer to one or several different steps, but in particular to the step of reading and/or measuring any kind of signal emitted or sent from the samples, wherein the signal will be interpreted as a hit or a non-hit, when a candidate agent is active or inactive, respectively, if compared to a control sample devoid of said agent.

The terms “automated”, “automatically” and “automation”, and the like, as used for example in the expression “automated process” or “in an automated way/manner”, refers to the fact that the respective step is conducted by any kind of machine or robot. Generally, such machines can be operated by and/or are under the control of a data processing machine, in particular a computer. Preferably, the data processing machine can be programmed to conduct specific steps in a specific way, as determined by the programmer and/or user. The term also refers to the fact that the respective step is not conducted by a human operator, for example by hand. Generally, the respective step is conducted for a number of different samples simultaneously and more preferably for a plurality of microtiter plates successively and possibly different steps are conducted for different microtiter plates simultaneously.

HTS, according to a preferred embodiment, may also involve at least at some stage and/or in at least one step the automated handling of a microtiter plate as disclosed herein, comprising a plurality of wells with samples.

According to an embodiment, besides the step of “signal reading”, also the step of interpretation of the signal may be conducted in an automated process, in particular by a data processing machine. Interpretation of the signal my involve the comparison of a given measured value with a corresponding control value, and the determination, in dependence of the presence or absence of substantial and/or significant difference between the compared values, that a candidate agent is or is not, respectively, a “hit”, that is, considered as an active agent.

According to an embodiment, one or several steps prior to the step of signal reading is/are conducted in an automated way. For example, one or more of the following steps are conducted in an automated manner: the step of adding and/or removing a buffer solution from a sample; the step of adding cells to the wells of the microtiter plate; the step of adding elements of or required for the reporting system to the sample; the step of adding a known standard agent of established effect to the sample, for example a positive control; the step of adding the candidate agent to be screened to a sample. The latter two may involve the addition of the respective agent in different concentrations, for example, in concentrations that vary from sample to sample; the step of removing solutions from the microtiter plate following the last measurement/sample reading; one or more steps of treating, handling and/or moving a plurality of microtiter plates successively. For example, microtiter plates may be transported from a stack where they are stored, incubated and/or placed. The different steps may be conducted at different times and may be coordinated as desired. For example, cells may be fed into plates and then a succession of microtiter plates may be stored, for example as one or more stacks, in a storage compartment. After a determined storage time, the microtiter plate may be subjected to testing and so on as mentioned. The steps cited above, can be made simultaneously for all wells of the microtiter plate. For example, a candidate agent, such as a candidate allosteric modulator, and/or a control substance or agent such as a known natural and/or a reported orthosteric ligand can be added to all samples (to all wells) at the same time.

According to a particular embodiment of the screening methods of the invention, the step of adding the candidate agent to be screened is conducted in an automated manner. According to an embodiment, a particular agent is added automatically in different concentrations to different samples. Preferably, the candidate agent is added to all samples of a microtiter plate at the same time and/or at a predetermined point in time. For example, this can be achieved by a robotic pipettor head, programmable to take up a programmable amount of liquid from one or more reservoirs into disposable tips and add the determined amount of liquid to the wells of a microtiter plate.

According to an embodiment, samples to be screened in the methods of the present invention are provided in a microtiter plate. The microtiter plate preferably harbors 50 or more, in particular 96 or more wells, preferably 300 or more, in particular 384 or more wells, more preferably 1000 or more, in particular 1536 or more wells, for example 3000 or more, in particular 3456 or more wells for an according number of samples. In each well, one sample can be provided. Since the present invention has the goal of providing rapid HTS, the method of the invention is preferably susceptible of processing/testing standard microtiter plates with 384 or more wells.

According to another embodiment, the microtiter plate harbors at least 100, more preferably at least 200, even more preferably at least 300, still more preferably at least 400 and most preferably at least 500 wells.

For the purpose of the present invention, one step of “signal reading”, “measuring”, “conducting a measurement”, “conducting measurements”, “measurement” in general, “measurement taking” and the like refers to the taking of as many individual and separate sub- or partial measurements as necessary and/or sufficient for the generation of a comparable and/or interpretable dataset. Depending on the reporting system used, such as molecular markers and the like, several measurements may be required in order to obtain an interpretable and/or comparable dataset. These several measurements are referred to herein as “partial” measurements, understanding that such a “partial” measurement as such is not incomplete, but needs to be combined with at least one further “partial” measurement in order to obtain said comparable and/or interpretable dataset. This is for example the case with FRET and BRET systems discussed further below, where light of specific, different wavelengths is generally measured successively for any individual sample in order to obtain a FRET or BRET ratio, respectively, wherein said ratio constitutes the interpretable and/or comparable dataset.

According to an embodiment, one step of signal reading and/or conducting measurements is conducted for all samples/wells of an individual microtiter plate at the same point in time, that is, simultaneously. If several successive partial measurements as discussed above have to be conducted, each individual partial measurement is conducted simultaneously for all wells of the microtiter plate.

Furthermore, steps of signal reading for each sample, and therefore, for each microtiter plate, are repeated several times over time. In other words, every individual sample is monitored over time. In this way, dynamic screening methods are obtained.

According to a preferred embodiment, the method of the invention comprises the steps of conducting, preferably in an automated manner, a series of repeated measurements, wherein said measurements are preferably repeated within determined time intervals.

The series of repeated signal reading and/or conducting measurements, in particular at the time intervals specified below, account for the dynamic properties of the screening methods of the present invention. Thanks to the series of repeated measurement, it is assured that a hit is detected even if the measurable signal and/or cellular response triggered by a candidate compound are very short or delayed, for example. The screening methods of the present invention thus allow for the identification of hits that would not be detectable with conventional screening methods, in particular those based on photomultiplier tubes that measure each well/sample only once or with repetitions following a comparatively long time period only.

According to a preferred embodiment, each sample is subjected to a step of signal reading for several times over a determined time span or period following addition of a candidate agent. Preferably, measurements are thus continuously made of each individual sample. According to an embodiment, the total period of repeated steps of measurements starting shortly (for example up to 10 seconds) before or at the moment of the addition of a candidate agent lasts up to 45, preferably up to 30, more preferably up to 15, and most preferably up to minutes. Preferably, measurements are conducted for at least 1, more preferably for at least 2, even more preferably for at least 3 and most preferably for at least 5 minutes. For examples, measurements on each sample are conducted for a time period of 2 seconds to 30 minutes, preferably 4 seconds to 15 minutes, and most preferably 6 seconds to 10 minutes.

According to an embodiment, measurements on each sample are repeated at intervals of 0.4 second to 10 minutes, 0.5 seconds to 5 minutes, 0.5 seconds to 2 minutes or 0.5 seconds to 1 minute, preferably 1 to 30 seconds, more preferably 1 to 15, even more preferably 1.1, 1.2 or 1.5 to 10, and most preferably 2 to 8 seconds. Preferably, measurements are made at regular intervals. The measurements may be repeated within determined time intervals of 2 seconds to 2 minutes.

The time (t) passed between the start of signal reading of a specific sample of a microtiter plate (followed by the reading of all samples of the plate) until the start of reading the same sample again is the “read frequency” of the assay. The read frequency is dependent on various factors such as the time spent of measuring for each sample, the number of partial measurements necessary for completing an interpretable dataset, adjustments necessary for taking a (partial) measurement. The read frequency can be expressed in seconds or, if expressed as 1/(t), in Hertz (Hz).

According to an embodiment, the frequency of reading is not longer than 10 seconds, preferably not longer than 6, more preferably not longer than 4, even more preferably not longer than 2, not longer than 1.5 seconds, and most preferably not longer than 1 second.

According to an embodiment, the read frequency is in the range of 0.4 to 8 seconds, preferably 0.5 to 7 seconds, more preferably 0.8 to 5, and most preferably 1.1 to 4.5 seconds.

Since, according to an embodiment, all samples of an entire microtiter plate are read simultaneously, the above frequencies apply to the entire microtiter plate, that is, all samples contained in the microtiter plate. The microtiter plate that can be used for the purpose of the present invention, for example those with 300 or more wells, are specified above.

As indicated above, the signal reading and/or conducting of measurements for each sample of an individual microtiter plate is preferably conducted in an automated manner and preferably simultaneously for the entire plate.

Of course, it is advantageous to conducting measurements at a high speed and at a high frequency. As the skilled person will understand, besides the sensitivity and speed of the signal reading system, the signal produced by the biological systems needs to be strong enough so that a relevant signal is captured in little time. As will become apparent from this specification, the present invention provides a reporting system that is capable of producing a strong signal that requires little time of signal reading by the signal reading system.

The signal produced by the reporting system and/or read during a step of signal reading is preferably electromagnetic radiation, and in particular light, for example visible light. For example, signal reading can involve the effect of absorption of light of a sample (extinction).

According to an embodiment, the signal read in a step of signal reading and/or conducting one or more measurements and/or in a partial measurement is a light signal. Light signals may be read, for example, by devices comprising light sensors, such as cameras.

Preferably, signal reading comprises the measurement of light of a specific wavelength or of one or more specific wavelength ranges, and more preferably the measurement of two or more distinct and/or different wavelengths and/or wavelength ranges. The two or more different wavelengths and/or wavelength ranges may be measured as two or more partial measurements as discussed above. The exact wavelength or wavelength range(s) measured in the step of signal reading depends on the reporting system used in the method of the present invention. Preferred reporting systems and their associated wavelengths and/or wavelength ranges suitable for interpretation will be discussed further below.

According to an alternative embodiment, all light emitted from a sample may be measured, recorded and/or directly analysed. The composition of the emitted light in terms of intensity at a specific wavelength or wavelength range may be done by the light sensing device or following recording of the light by the sensing device. For example, one can envisage a color photograph (or other type of recording or registration) being taken and being analysed following taking of the photograph (recording, registration), or possibly directly while sensing light emission, directly registering the wavelength distribution of the light emitted from a sample. According to an example, a camera is used, which contains pixels that only collects light from one light emitting entity (e.g. luminescent protein) and other pixels that collect light from the other light emitting entity (e.g. fluorescent protein).

Devices capable of performing simultaneous signal reading from an entire microtiter plate are commercially available. For measuring light signals, for example, a camera may be used to take a picture of an entire microtiter plate, in particular of every well of the microtiter plate. In this way, light emission of all samples in the wells of an entire microtiter plate can be measured at the determined time intervals simultaneously.

In HTS, devices using charge-coupled devices (CCDs) may be used, for example a CCD camera. According to an embodiment, the signal measurement, for example, measurement of light emission is determined by an intensified charge coupled device (ICCD) camera. For example, an ICCD camera may take a photo of an entire microtiter plate. Preferably, the ICCD camera intensifies the signal and calculates signal (in particular light) units per well.

Such cameras thus capture non-amplified photons, transform them to electrons, and use the electrons to amplify the signal. While ICCD cameras do generally not possess the same sensitivity as photomultiplier tubes frequently used in the prior art, the former possess the advantage that electromagnetic radiation of all wells of a single microtiter plate can be measured at the same point in time, while with the latter measurements are conducted for one well after the other, or at best for a row or column of the microtiter plate at a time. The method of the present invention, providing the step of signal reading for the entire microtiter plate simultaneously thus represents an important advantage of speed and of the dynamic characteristics of the methods of the invention. In addition, as set out above, one or more steps may be conducted in an automated manner, for example during reading time. For example, a test or candidate agent can be added to each well, or different test or candidate agents can be added to all wells, at the same time, and, according to an embodiment, simultaneously as signal is measured. This allows the direct measurement of the effects of adding compounds to the wells.

It is an objective of the present invention to provide a dynamic screening assay. In other words, the potential effect of a candidate agent on a target receptor is preferably monitored over time in order to also identify candidate agent that only have a short, delayed or little activity on the target receptor. Therefore, measurements are preferably conducted on a living biological system containing the receptor. According to a preferred embodiment, the biological system comprises living cells, as discussed in more detail further below. Preferably, the methods of the invention, in particular the step of taking measurements, are non-destructive and/or non-invasive.

The cells of the present invention preferably harbor, produce and/or express a reporting system. The reporting system, reports a signal that can be interpreted and assigned to a state of activity of a target receptor and/or the presence or absence of signal transduction.

Since, as mentioned above, the step of signal reading and/or conducting measurements preferably comprises the step of reading a light signal, the reporting system preferably is a light producing, emitting, reflecting and/or absorbing system. Preferably, light emission from the cells harboring the system can be measured. The signal emitted by the reporter system is preferably sufficiently strong to differentiate over background or noise signals emitted from the cells as such and/or possibly resulting from illumination of the cells. The present invention in particular addresses the problem of providing a reporter system that emits a sufficiently strong and long signal so as to permit the detection of comparatively low or little variations with respect to the binding of an agent to the receptor and/or the activity state of the receptor. The present invention in particular provides a reporter system that is sensitive, in particular sensitive enough to also detect signals emitted due to little variations of receptor activity resulting from the action of an allosteric modulator or of a low affinity active agent, for example.

According to an embodiment of the invention, a chimeric receptor is used, which incorporates at least part of the reporting system.

According to an embodiment, the light produced by the reporting system is fluorescent and/or bioluminescent light.

It is also reconsidered, at this position, that it is an objective of the present invention to provide proximal screening methods. The reporting system thus produces a signal upon an early event in the process of receptor activation and/or modulation and signal transduction. Preferably, the reporting system is also capable of reporting a modulation of the receptor activity.

The reporting system is preferably based on one or more chimeric proteins of the invention. For the purpose of the present specification, the reference to the proteins also includes reference to the cells containing the protein(s), to nucleotide sequences encoding the protein(s), to cells expressing one or more nucleotide sequence encoding the protein(s), cells containing one or more expression vectors comprising the nucleotide sequences, the expression vectors as such, and so forth. The cells, protein(s), vectors, nucleic acids comprising the nucleotide sequences disclosed in this specification may be provided in isolated form.

According to an embodiment, said reporter system comprises at least a chimeric protein (a) comprising a GPCR fused to a Gα subunit and also fused to one selected from a luminescent protein and a fluorescent protein, and/or a chimeric protein (b) comprising a Gγ and/or a Gβ subunit fused to one selected from a luminescent protein and a fluorescent protein. In chimeric proteins (a) and (b), the order in which the indicated components (e.g. GPCR, Gc subunit, luminescent or fluorescent protein) are fused to each other is not relevant and all combinations are explicitly encompassed by the present invention.

In other words, the chimeric protein (a) comprises an amino acid sequence of a GPCR fused to the amino acid sequence of a Gα subunit and also fused to an amino acid sequence of one selected from a luminescent protein and a fluorescent protein.

Accordingly, the chimeric protein (b) comprises an amino acid sequence of a Gγ and/or a Gβ subunit fused to an amino acid sequence of one selected from a luminescent protein and a fluorescent protein.

Specific examples of GPCRs, Gα, Gγ and/or Gβ subunits are given further below. Preferably, nucleotide or amino acid sequences of human or animal GPCR Gα, Gγ and/or a Gβ subunit are, independently used. However, the present invention covers, independently, the use of functional GPCRs, Gα, Gγ and/or a Gβ subunits having, independently, at least 50%, 55%, 60%, 65% 70%, 75%, 80%, preferably at least 85%, 90%, 91%, 92%, 93%, 94%, 95%, 96%, 97%, or at least 98%, or 99% amino acid sequence identity with a known or yet to be discovered GPCR, Gα, Gγ and/or a Gβ subunit sequence, respectively. Preferred identity levels and the method for determining sequence identity as discussed further below applies. The present invention also encompasses GPCRs, Gα, Gγ and/or a Gβ subunits having amino acid sequences of functional variants, as defined below, of known or yet to be discovered GPCRs, Gα, Gγ and/or a Gβ subunits. The above sequence identities apply to each one of GPCR, Gα, Gγ and/or a Gβ independently. Preferably they apply specifically to the GPCR. According to an embodiment, they apply to any one or both of the Gα and Gγsubunits. According to an embodiment, said GPCR is functional, meaning that it retains the capacity of being activated by its orthosteric and allosteric ligands, in particular by the natural orthosteric ligand. The capacity of being activated of a construct comprising a mutated GPCR can be assessed by the method of the invention, using the original or natural GPCR sequence.

Linkers, targeting sequences, and/or other amino acid moieties and/or peptides having or not having a specific function, may be, independently, comprised within or an the N- and/or C-terminal ends of the chimeric proteins (a) and/or (b), independently. Amino acid sequences encoding the linkers, targeting sequences, and other peptides may be flanking the sequences of the GPCRs, Gα, Gγ and/or a Gβ subunits, fluorescent or luminescent protein, or they may be provided between two functional proteins, for example between the amino acid sequence encoding a GPCR and the one encoding a Gα subunit. It is also possible, in particular with the GPCRs, Gα, Gγ and/or a Gβ subunits, that sequences of the latter are interrupted and another functional protein, for example a fluorescent and/or luminescent protein, is situated within the amino acid sequence of one or more of GPCR, Gα, Gγ and/or a Gβ. Accordingly, the expression “fused to” is not intended to be limited to “directly fused to”, but also encompasses the possibility that different amino acid residues or entire sequences are situated between two or more protein fused one to the other. According to an embodiment, in said protein (a), said Gα subunit is fused in a continuous manner to said GPCR. According to another embodiment, in said protein (a), said Gα subunit is split in at least two separated sequence parts fused to said GPCR.

Preferably, if said chimeric protein (a) comprises a luminescent protein, the chimeric protein (b) comprises a fluorescent protein, and if chimeric protein (b) comprises a luminescent protein, (a) comprises a fluorescent protein.

According to an embodiment, said chimeric protein (a) is selected from the following chimeric proteins:

i) GPCR—Gα—luminescent protein;
ii) GPCR—Gα—fluorescent protein;
iii) GPCR—luminescent protein—Ga;
iv) GPCR—fluorescent protein—Ga;
v) GPCR—split Gα part I—luminescent protein—split Gα part II;
vi) GPCR—split Gα part I—fluorescent protein—split Gα part II;
wherein the protein components (GPCR, Gα, luminescent protein, fluorescent protein, split Gα part I, split Gα part II) of the indicated chimeric proteins are present in the indicated order in said chimeric protein, and wherein amino acid moieties or sequences having or, independently, not having further functionalities may be provided terminally and in positions indicated with “-”.

With respect to v) and vi), it is noted that that position of the insert of the luminescent or fluorescent protein within the Gα sequence can vary. The only requisite will be for the fusion protein to remain functional, that is, the G protein activity remains unchanged in spite of the split in two separate parts interrupted by another sequence.

According to an embodiment, said chimeric protein (b) is selected from the following chimeric proteins:

vii) Gγ—luminescent protein;
viii) Gγ—fluorescent protein;
ix) luminescent protein—Gγ;
x) fluorescent protein—Gγ;
wherein the protein components (Gγ, Gα, luminescent protein, fluorescent protein) of the indicated chimeric proteins are present in the indicated order in said chimeric protein, and wherein amino acid moieties or sequences having or, independently, not having further functionalities may be provided terminally and in positions indicated with “-”.

For example, in the embodiments mentioned above, linker amino acid sequences may independently be provided at positions indicated with “-”.

Preferably, in i), ii), iii), iv), v), vi), xi), and xii) and/or in vii), viii), ix), x), xiii), xiv), xv), and xvi) proteins are shown from the N (on the left) to the C terminus (on the right).

According to an embodiment, the invention provides a nucleotide sequence encoding, in this order from 3′ to 5′ or 5′ to 3′, a chimeric protein (a) selected from i), ii), iii), iv), v), vi), xi) and xii) as defined above and below.

According to an embodiment, the invention provides a nucleotide sequence encoding, in this order from 3′ to 5′ or 5′ to 3′, a chimeric protein (b) selected from vii), viii), ix), x), xiii), xiv), xv), and xvi) as defined above and below.

It is noted also that the fluorescent and/or bioluminescent proteins may, independently be split in partial sequences as shown with respect to the examples of v) and vi) above. Accordingly, said chimeric protein (a) may further be selected from:

xi) GPCR—split Gα part I—split luminescent protein part I—split Gα part II—split luminescent protein part II;
xii) GPCR—split Gα part I—split fluorescent protein part I—split Gα part II—split luminescent protein part II.

Similarly and independently, said chimeric protein (b) may be selected from the following chimeric proteins:

xiii) Split Gγ part I—split luminescent protein part I—Split Gγ part II— split luminescent protein part II;
xiv) Split Gγ part I—split fluorescent protein part I—Split Gγ part II— split fluorescent protein part II;
xv) Split luminescent protein part I—split Gγ part I—Split luminescent protein part II—split Gγ part II;
xvi) Split fluorescent protein part I—split Gγ part I—Split fluorescent protein part II—split Gγ part II.

With respect to the “-” and to the preferred orientation (3′ to 5′, 5′ to 3′) the same as above applies also independently to x)-xvi).

According to an embodiment, the nucleotide sequence of the invention comprises the sequence parts encoding the individual proteins and/or polypeptides as detailed above and elsewhere in this specification, including modified polypeptides preferably having the indicated sequence identity levels, and possibly additional marker, linker or other functional peptide sequences. The order in which the individual sequence parts are linked to each other is not relevant as long as a functional polypeptide is obtained, which can be used for the purpose of screening in accordance with the present invention.

According to an embodiment, in the nucleotide sequence of the invention, said nucleotide sequence parts (encoding fully or partially a GPCR, Gα, luminescent protein, fluorescent protein, Gγ, Gβ, etc) are translationally fused in any order to the respective other sequence parts (for example, the GPCR to the Gα and further to a luminescent protein or fluorescent protein), “Translationally fused” means that the nucleotide sequence when translation in protein biosynthesis yields a single, continuous polypeptide comprising amino acid sequences encoded by said sequence parts. In the encoded polypeptide, any functional protein (GPCR, Gα, luminescent or fluorescent protein; Gγ or Gβ and fluorescent or luminescent protein) may be interrupted by intermittent amino acids or peptide sequences, on the basis of a coding region or ORF of said nucleotide sequence.

According to an embodiment, said Gα can be any known or yet to be discovered Gα-subunit, such as Gαₛ, Gα_{i/o, Gα}_{q}, Gα₁₁, Gα₁₂, Gα₁₃, Gα₁₄, Gα₁₅, Gα₁₆, Gα_{T} and Gα_{Z} G-protein subunit, for example.

According to an embodiment, the Gα is selected from Gαₛ (Gas), Gα_{q} (Gαq) and Gα_{i/o} (Gαi/o). According to an embodiment, since specific GPCRs use specific Gα protein for signalling, the above selection of preferred Gα proteins also entails a selection to the GPCRs using these Gα proteins, or even GPCRs using these Gα proteins as the preferred Gα subunit, in case more than one Gα subunits are used.

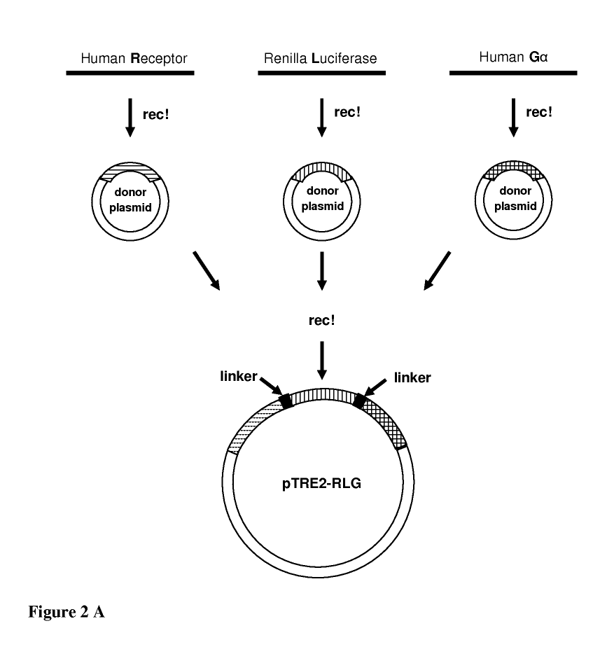

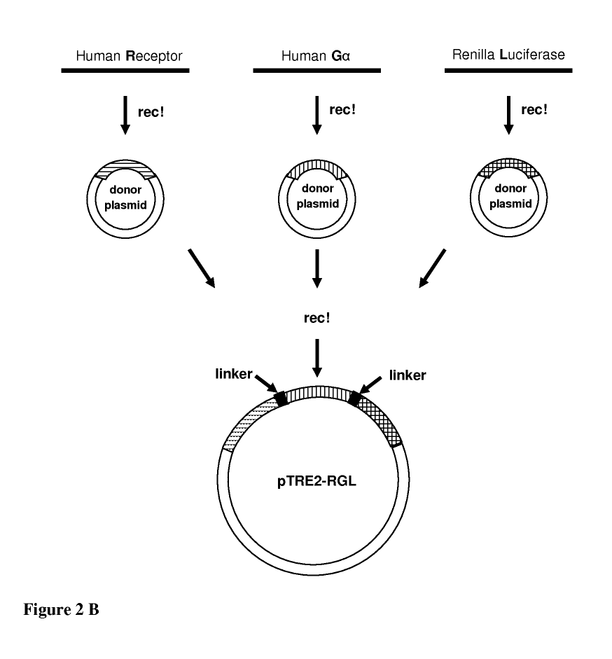

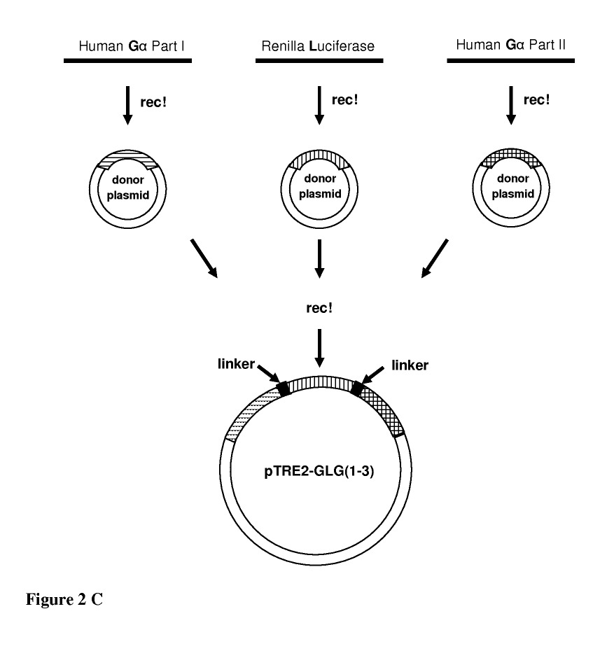

In accordance with the exemplary embodiments v) and vi), several examples in which a Gα protein is provided in a split form on said chimeric protein, with the luminescent protein (or fluorescent protein) being provided within the two parts (part I, part II) of the Gα protein. It is noted that a split for separating the two parts may, in principle be made at any position, as long as a functional chimeric polypeptide is obtained. In the examples shown (GLG1-3, FIG. 2C), the Gα protein is split at one of the loop domains, as may be identified by looking at protein crystal structures or sequence analysis, for example. In general, a split has to be such that correct folding of the protein or its separated parts that are part of the chimeric protein can fold correctly.

According to an embodiment, said Gγ can be any known Gγ subunit, from Gγ1 to Gγ13 G-protein subunit. For example, the Gγ subunit may be selected from any one of the following Gγ subunits isolated from Homo sapiens and identified by their accession numbers, as well as the homologues in other species and sequences being substantially identical to the following: NM_{—}021955, NM_{—}053064, NM_{—}012202, NM_{—}004485, NM_{—}005274, NM_{—}052847, NM_{—}033258, NM_{—}001017998, NM_{—}004126, NM_{—}018841, NM_{—}016541.

According to an embodiment, the fluorescent entity is a fluorescent protein.

According to an embodiment, the fluorescent protein is a yellow fluorescent protein (YFP). Many different YFPs are disclosed in the prior art and indeed may be suitable for practicing the present invention. Best results, however, producing clear signals in particular also in case of screening for allosteric modulators, are found when a YFP comprising one, several or all mutations selected from the group of F46L, 147L, F64L, R79K, M153T, V163A, S175G, S208F, S208F, V224L, H231E and D234N, if aligned with YFP derived from green fluorescent protein (GFP) isolated from Aequorea victoria jellyfish and having the mutations S65G, S72A K79R, T203Y. The original gene is green fluorescent protein (GFP) that was isolated from Aequorea victoria jellyfish (Accession number CAA58790.1).

According to an embodiment, the FP is a yellow FP(YFP) and comprises one, two, three or all four mutations selected from S65G, S72A, K79R, T203Y if aligned with CAA58790.1. In addition, the FP used in the BRET-based assay of the invention comprises, if aligned with Aequorea victoria green-FP (Accession number CAA58790.1), at least one, two, three, four, five, six, seven, eight, nine, ten, eleven, or all mutations selected from the group of F46L, 147L, F64L, R79K, M153T, V163A, S175G, S208F, S208F, V224L, H231E and D234N. In particular, the mutant FP of the present invention comprises at least the mutations F46L, 147L, S208F, V224L, H231E and D234N. According to an embodiment, the FP of the invention comprises the mutations F64L, R79K, M153T, V163A, S175G, which are also present on YFP venus.

YPF is mutated from GFP and the preferred YFP used in accordance with the invention is a mutated form of YFP as disclosed in Nguyen and Daugherty (see below).

According to an embodiment, the fluorescent protein is a yellow fluorescent protein comprising at least one amino acid sequence selected from (i) an amino acid sequence encoded by the nucleotide sequence of SEQ. ID. NO.: 2, (ii) an amino acid sequence having at least 70%, preferably at least 80%, 85%, 90%, 95%, 96%, 97%, 98%, or at least 99% identity with the amino acid sequence of (i), and (iii) a functional variant of (i) or (ii). The fluorescent protein according to this embodiment preferably comprises one or more of the (preferred) mutations according to the embodiments specified for fluorescent proteins elsewhere in this specification.

The YFP encoded by SEQ. ID. NO.: 2, which is particularly useful for the purpose of the present invention, is also known as YPet and is disclosed by A. Nguyen and P. Daugherty, in “Evolutionary optimization of fluorescent proteins for intracellular FRET”, Nature Biotechnology, vol. 23, no. 3, pp. 355-360.

According to an embodiment, said fluorescent protein accepts light of wavelengths having a peak lying within the range of 490-530 nm (acceptor wavelength) and emits fluorescent light of wavelengths having a peak lying within the range of 500-560 nm (emission wavelength). Preferably, said fluorescent protein accepts light having a peak within the range of 500-520 nm and emits light of wavelengths having a peak lying within the range of 520-540 nm.

Preferably, in the fluorescent protein, the acceptor and emission wavelengths have peaks of maximum intensity that lie apart, at different wavelengths. More preferably, the acceptor and donor wavelength peaks are at least 10, more preferably at least 15 nm apart.

According to an embodiment, the expression “fluorescent protein” also encompasses proteins that are by themselves not fluorescent but which may be modified to be fluorescent, for example by fluorescent labelling, like SNAP-tags. Accordingly, the expression “fluorescent protein” also encompasses “fluorescent labels that can be bound, for example covalently, to a protein”.

According to an embodiment of the invention, the fluorescent protein may be replaced by a fluorescent entity, for example a fluorescent label, and the entity may be attached directly to the protein otherwise being fused to the fluorescent protein. To illustrate this principle, instead of a FP-Gγ, Gγ-FP, Gβ-FP, FP-Gβ protein, a FE-Gγ protein or modified protein may be used, where FE is any fluorescent entity, such as small molecules, that can have fluorescent properties. Other references of “fluorescent protein” or “FP” may thus be replaced, in an alternative embodiment, by fluorescent entity (“FE”) in general. For example, the chimeric protein (a) may comprise a GPCR fused to a Gα protein and further connected to a FE. The FE may be connected to the GPCR or to the Gα protein. The expression “connected”, in the context of the FE encompasses any kind of association, such as binding by a covalent bond, electrostatic forces, hydrophobic attractive forces, hydrogen bridges, van der Waals forces or other bonds.

Examples of FE are SNAP-, CLIP-, ACP- and MCP-tags, for example (www.neb.com). Using these tags, fluorescent molecules (tags) may be bound to a target of interest, such as chimeric protein (a) or protein (b).

Bioluminescent proteins have been isolated from a variety of different organisms and are available to the skilled person. Exemplary luminescent proteins are luciferases and photoproteins. Examples of luciferases are the firefly luciferase of Photinus pyralis, the Renilla luciferase of Renilla reniformis (RLuc), and the Metridia luciferase of Metridia longa (MetLuc), for example. Mutated luciferases with different properties were created and may be used as well for the purpose of the present invention. According to an embodiment, a wild-type or mutated Renilla luciferase is used.

According to an embodiment, said luminescent or bioluminescent protein is a luciferase, for example a luciferase selected from a luciferase (RLuc) present in Renilla reniformis and mutants thereof. Preferably, said luminescent protein is a mutant luciferase comprising, if aligned with a luciferase (RLuc) present in Renilla reniformis, one, several or all of the mutations selected from the group of: A55T, C124A, 5130A, K136R, A143M, M185V, M253L and S287L. Luciferases of this type are particularly advantageous for the purpose of the invention, as they combine well with the preferred fluorescent protein mentioned above. Preferably, said luciferase (RLuc) present in Renilla reniformis is the sequence with Accession number AAA29804.1/GI 160820).

According to an embodiment, the mutant luminescent protein comprises at least the mutation C124A. According to an embodiment, the mutant luciferase comprises at least the mutation M185V. According to an embodiment, the mutant luciferase comprises at least the mutations C124A and M185V. Furthermore, the luciferase preferably comprises one or more further mutations selected from A55T, 5130A, K136R, A143M, M253L and S287L. According to an embodiment, the mutant luciferase comprises at least the mutation S287L, preferably the mutations C124A and S287L, more preferably the mutations C124A, S287L and M185V, besides, optionally, one or more mutations selected from A55T, 5130A, K136R, A143M, and M253L. According to a preferred embodiment, the mutant Renilla reniformis is the luciferase RLuc8 (Andreas Markus Loening, Timothy David Fenn, Anna M. Wul and Sanjiv Sam Gambhir Protein Engineering Design and Selection 2006 19(9):391-400)

According to a preferred embodiment, the luminescent protein comprises at least one amino acid sequence selected from (i) an amino acid sequence encoded by the nucleotide sequence according to SEQ. ID. NO.: 1, (ii) an amino acid sequence having at least 70%, preferably at least 80%, 85%, 90%, 95%, 96%, 97%, 98%, or at least 99% identity with the amino acid sequence of (i), and (iii) a functional variant of (i) or (ii). The luminescent protein according to this embodiment preferably comprises one or more of the (preferred) mutations according to the embodiments specified for luminescent proteins elsewhere in this specification. The luciferase encoded by the nucleotide sequence of SEQ. ID. NO.: 1 is particularly useful for the purpose of the present invention, in particular when used in combination with the preferred embodiments of the fluorescent protein disclosed above.

It is noted that the amino acid sequence encoded by nucleotide sequence of SEQ ID NO: 1 contains a C terminal Sigma®'s FLAG® Tag encompassing the last nine amino acid moieties. This tag is also present in other sequences encoding or containing the luciferase encoded by SEQ. ID. NO.: 1 (e.g. SEQ. ID. NO.: 7-24, 83, 84, 86-101). This allows for antibody detection, but has no purpose with respect to the functionality of the screening assay as such. Preferably, this tag is not considered for the purpose of sequence identity determination and sequence comparison. On the other hand, this tag is an example of terminal or intermediate sequences that may be present within the scope of the nucleotide and amino acid sequences of the invention.

According to an embodiment, said luminescent protein emits light of wavelengths having a peak lying within the range of 420 and 520 nm, preferably within the range of 440-500, and more preferably within the range of 470-490. Preferably, said luminescent protein emits light over a large wavelength range. For example, the luminescent protein emits light in the wavelength range of 500-520 nm at ⅓ or more of the intensity of light emitted at the wavelength peak (maximum intensity of the luminescent protein). Preferably, the luminescent protein emits light in the wavelength range of 490-510 nm at an intensity corresponding to ½ or more of the maximum intensity.

According to an embodiment, the chimeric protein (a) comprises at least one selected from (i) an amino acid sequence of one selected from any one of SEQ. ID. NO.: 8, 10, 12, 14, 16, 18, 20, 22, 24, 84, 87, 89, 91, 93, 95, 97, 99, and 101; (ii) an amino acid sequence having at least 70%, preferably at least 80%, 85%, 90%, 95%, 96%, 97%, 98%, or at least 99% with any one of SEQ ID. NO.: 8, 10, 12, 14, 16, 18, 20, 22, 24, 84, 87, 89, 91, 93, 95, 97, 99, and 101; and (iii) a functional variants of any one sequence of (i) or (ii).

Nucleotide sequences encoding exemplary chimeric proteins (a) of the invention may be selected from the sequences given in SEQ. ID. NO.: 7, 9, 11, 13, 15, 17, 19, 21, 23, 83, 86, 88, 90, 92, 94, 96, 98 and 100.

According to an embodiment, the chimeric protein (b) comprises at least one selected from (i) an amino acid sequence selected from any one of SEQ. ID. NO.:4 and 6, or a Gγsequence selected from any one of any one of SEQ. ID. NO.: 102-112, or of the Gγ9 sequence contained in any one of SEQ. ID. NO.:4 and 6; (ii) an amino acid sequence having at least 70%, preferably at least 80%, 85%, 90%, 95%, 96%, 97%, 98%, or at least 99% identity with any one of SEQ. ID. NO.: 4 and 6, or a Gγ sequence selected from any one of any one of SEQ. ID. NO.: 102-112, or of the Gγ9 sequence contained in any one of SEQ. ID. NO.:4 and 6; and (iii) a functional variant of any one sequence of (i) or (ii).

Nucleotide sequences encoding exemplary chimeric proteins (b) of the invention may be selected from the nucleotide sequences SEQ. ID. NO.: 3 and 5. Further exemplary chimeric proteins (b) may be selected from fusions of any one of SEQ. ID. NO.: 102-113 (but also of Gγ9) with any one selected from a fluorescent and a luminescent protein in accordance with the invention, for example one selected from SEQ. ID. NO: 1 (luminescent protein) and 2 (fluorescent protein). The fluorescent and luminescent protein may be fused to the N-terminus or the C-terminus of the encoded polypeptide (any one of the 5′ or 3′ end with respect to the nucleotide sequence). Linkers and other functionalities may be present. It is also possible that sequences (e.g. of any one selected from the Gγ, of the fluorescent protein and of the luminescent protein) are split up in partial sequences and fused in this way to each other, as long a functional construct and chimeric protein (b) is obtained.

According to a preferred embodiment, the proteins used for the purpose of the present invention have, independently, at least 60%, more preferably at least 70%, even more preferably at least 75%, 80%, 85%, 90%, 91%, 92%, 93%, 94%, 95%, 96%, 97%, 98% or 99% identity with any one of the original amino acid sequences disclosed herein, such as those listed in the sequence listing that is part of the present application. These percentages may be selected independently for any peptide or amino acid sequence as defined herein.

Amino acid sequence identity is preferably determined by using the basic protein blast on the internet (http://blast.ncbi.nlm.nih.gov) with preset standard parameters and database selections. This sequence comparison tool is based on algorithms detailed in the two following publications: Stephen F. Altschul, Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. Stephen F. Altschul, John C. Wootton, E. Michael Gertz, Richa Agarwala, Aleksandr Morgulis, Alejandro A. Schäffer, and yl-Kuo Yu (2005) “Protein database searches using compositionally adjusted substitution matrices”, FEBS J. 272:5101-5109.

Standard parameters include the selection of blastp (protein-protein BLAST, automatic adjustment of parameters to short input sequences; expect threshold 10, word size 3, use of the matrix BLOSUM62; Gap costs: existence: 11, extension 1; conditional compositional score matrix adjustment, no filters and no masking).

Sequence identity of a sequence of comparison with respect to an original sequence is reduced when, for example, any one of the compared or the original sequence lacks amino acid residues, has additional amino acid residues and/or has one or more amino acid residue substituted by another residue. Sequences having as little as 60% sequence identity with any sequence as defined herein may still provide functional, that is, having receptor functionality, fluorescent functionality, luminescent protein functionality, G protein (α, β and/or γ) subunit functionality and/or modulating functionality, and are thus suitable to meet the objectives of the invention.

A functional variant, for the purpose of the present specification, mainly covers the following situations.

First, variants relate to peptides comprising amino acid sequences as defined in this specification, which sequences contain insertions and/or deletions encompassing at least three but possibly more amino acids. Such deletions and/or insertions may have no effect on the functional properties of the peptides, but may be used to provide further, other or modified functionalities to the peptide as such. Such insertions and/or deletions of larger scale may to a large extent affect the sequence identity so that the latter is rendered meaningless, even if the functional properties of the sequences disclosed herein are exploited. Therefore, the present invention also encompasses such variants. According to an embodiment, the variant includes, independently one from the other, zero, one, two, three, four or more insertions and/or deletions encompassing continuous stretches which independently may extend over three or more continuous amino acids, preferably 4 or more, 5, 10, 15, 20, 30, 40, 50, 60, 70, or more continuous amino acids. For example, the insertion and/or deletion may extend to up to 5, 10, 20, 30, 40, 50, 60, 70, 80, 90, 100, 120, 150, 170, 200 amino acid residues.

In addition, variants refer to substitutions of entire regions, for example of the same number of continuous amino acids as the continuous stretches mentioned above, with stretches having similar hydrophobicity characteristics, wherein several amino acids in a row are replaced by a residue having similar physiochemical characteristics. Examples of conservative substitutions include substitution of one aliphatic amino acid residue for another, such as Ile, Val, Leu, or Ala for one another, or substitutions of one polar residue for another, such as between Lys and Arg; Glu and Asp; or Gln and Asn. See Zubay, Biochemistry, Addison-Wesley Pub. Co. (1993). The effects of such substitutions can be calculated using substitution score matrices such as PAM-120, PAM-200, and PAM-250 as discussed in Altschul (J. Mol. Biol. 219:555-65, 1991). Conservative substitutions are more frequently present in domains or positions of the amino acid sequence that are outside or do not constitute the active pocket or site of the protein, but they may also be found in this region.

Variants comprising continuous stretches of conservatively substituted amino acids are separately considered here because such substitutions can have an important impact on the identity level of compared to an original sequence as defined herein, so that the figures of identity indicated above would be rendered meaningless to some extent. Accordingly, in an embodiment, conservatively substituted amino acid residues may make up to 50%, preferably however 40% or less, 30, 20, 15, 10, 9, 8, 7, 6, 5, 4, 3, 2, 1% or less of the amino acid sequences disclosed herein.

Further variants are, for example, naturally peptide variants that result from alternate mRNA splicing events or from proteolytic cleavage of the peptides described herein. Variants attributable to proteolysis include, for example, differences in the N- or C-termini upon expression in different types of host cells, due to proteolytic removal of one or more terminal amino acids from the polypeptides encoded by the sequences of the invention.

Fusion peptides and/or proteins may also be encompassed by the variants for the purpose of the present invention. Fusions of additional peptide sequences at the amino and/or carboxyl terminal ends of the chimeric polypeptides of the invention may be used to enhance expression and/or extracellular secretion and/or may aid in the purification of the protein, for example. For example, peptides as defined herein further comprising a signal peptide and/or a His-tag and/or a different tag fulfilling any specific function are also encompassed by the present invention.

According to an embodiment, said protein (a) and said protein (b) can exist in said cells in a first state, where there is no or little energy transfer, and a second state, where there is a substantial energy transfer from the luminescent protein or fluorescent protein of chimeric protein (a) to the luminescent protein or fluorescent protein of chimeric (b), or vice versa, wherein said energy transfer affects the quantity of measurable light emitted by said fluorescent protein(s) and/or said luminescent protein.

Said first and second states depend, in an embodiment, on the close physical proximity and/or favorable orientation or absence of proximity and/or unfavorable orientation, respectively, of (a) with respect to (b), and/or, more specifically, of said luminescent protein or fluorescent protein of chimeric protein (a) with respect to the luminescent protein or fluorescent protein, respectively, of chimeric protein (b).

The expressions “first state” and “second state” preferably do not imply any order or priority of the two states. These expressions express the presence or absence of energy transfer. These terms may, for example, be replaced by “energy transfer state” (for example BRET), corresponding to basal state, and “no-energy transfer” (for example absence of BRET), corresponding to activated state, respectively.

Furthermore, these states are, according to an embodiment, not absolute values but relative values and the method of the invention is suitable to detect relative changes of energy transfer. Therefore, the expression “a first state, where there is no or little energy transfer, and a second state, where there is a substantial energy transfer” can also preferably be reformulated to state “a first state, where there is no or little energy transfer and a second state, where there is more energy transfer than in said first state”. Another formulation would be: “a first state, where there is a first (amount, intensity of) energy transfer and a second state, where there is a second (amount, intensity of) energy transfer”. In this latter case, an agent is considered an active agent if the difference between said first and second energy transfers are detectable and/or this difference is above a threshold difference value. Such a threshold value may be determined beforehand.

Said energy transfer is preferably resonance energy transfer. The reporting system may thus be preferably selected from a FRET and a BRET system. Preferably, said energy transfer is bioluminescence resonance energy transfer (BRET). BRET is considered advantageous over Förster resonance energy transfer (FRET) for the purpose of the present invention. FRET measurements are tedious. The requirement for the excitation of donor molecules produces a range of problems including cell damage, photobleaching, low signal-to-noise ratios due to intrinsic cellular autofluorescence, and in particular direct excitation of the acceptor molecule.

Although the use of BRET system is preferred for the purpose of the present invention, the use of a FRET system may also be encompassed by the present invention. In this case, two fluorescent proteins are used, one of them being part of chimeric protein (a) and the other part of the chimeric protein (b).

Therefore, according to a preferred embodiment, one selected of chimeric protein (a) and protein (b) comprises a luminescent protein and the respective other comprises a fluorescent entity, for example a fluorescent protein. According to a preferred embodiment, proteins (a) and (b) each comprise only either one luminescent protein or one fluorescent entity or protein. In order for a BRET system to work, it is not relevant which of (a) and (b) comprises the luminescent protein and which comprises fluorescent entity, e.g. protein. In the embodiments shown in the examples, chimeric protein (a) comprises the luminescent protein.

If protein (b) comprises a fluorescent protein or a luminescent protein, protein (b) is a chimeric protein (b).

According to an embodiment, in the step of reading a signal emitted from the sample/cells, in particular light, light emitted by the luminescent protein or the fluorescent protein comprised in protein (a) (hereinafter for reasons of convenience more shortly referred to as: light emitted by (a)) and light emitted by the luminescent protein or the fluorescent entity, for example protein comprised in (b) (hereinafter: light emitted by (b)) is determined specifically and/or separately, and a ratio expressing the relative amount of light units, such as light intensity, emitted by (a) with respect to the light units, such as light intensity, emitted by (b), or vice versa, is determined for each sample. The ratio of light emitted by (a) with respect to (b), or vice versa, is generally referred to as the BRET ratio, which changes with the change from said first to said second state and vice versa, and, when determined thus constitutes an interpretable signal and/or dataset.

For example, during the step of light measurement, an emission filter is used, which lets only or to a larger extent the light of (a) or (b), respectively, passing, or, alternatively, which filters light emitted principally from (a) or (b), respectively. According to an embodiment, two emission filters are used, a first filter letting at least some light of (a) pass and a second emission filter letting at least some light of (b) pass. Preferably, the first filter does let no or only little (e.g. 20%) light of (b) pass and the second filter does no or only little (e.g. 20%) light emitted by (a) pass. According to an embodiment, a first filter is used that filters high-intensity light emitted by (b) and a second filter filters high-intensity light emitted by (a).

Accordingly, during a measurement step, it may be necessary to change the emission filters and to take measurements twice, in two partial measurements, once with each emission filter. In this way, a ratio of light emitted by (a) to light emitted by (b) can be determined and an interpretable dataset is produced.

Generally, each respective emission filter lets light of a wavelength or wavelength range pass that encompasses the emission peak and/or light of a wavelength range encompassing the wavelength were the strongest, most intense light signal of the luminescent protein or fluorescent entity of (a) and/or of (b), respectively, is produced. In this way, a strong light signal of the luminescent protein and/or fluorescent entity of (a) or (b), respectively, but not of both, can pass the respective emission filter.

According to an embodiment, the luminescent protein and the fluorescent entity comprised in chimeric protein (a) and protein (b) are selected in order to provide an optimal BRET pair, providing light signals that can easily be separated and/or separately analysed. In particular, the acceptor wavelength range of the fluorescent protein corresponds in a widest possible range to the donor wavelength emitted by the luminescent protein, or, in case of a FRET pair, to the donor fluorescent entity, for example protein.

Preferably, the acceptor wavelength peak and/or range of the fluorescent protein are close to the emission wavelength peak of the luminescent protein. Furthermore, the emission wavelength peak of the fluorescent protein is further away from the emission wavelength peak of the luminescent protein than the acceptor wavelength peak of the fluorescent protein.

The principles indicated above with respect to a BRET system also apply to FRET systems analogously. In this case, one has to distinguish between a first fluorescent protein, which takes the role of the luminescent protein of a corresponding BRET system, and which, in said second state, transfers energy to a second fluorescent protein.

In the present invention, the activation mechanism of a GPCR generally involving dissociation of the G protein subunits from the receptor and the dissociation of the G protein subunits Gα, Gβ and Gγ from each other is advantageously exploited. When the Gγ or Gβ subunit dissociates from the Gα subunit, the reporting system produces a change in BRET ratio, in particular a decrease of BRET between the fluorescent and bioluminescent protein components of the chimeric protein (a) and protein (b). In this way, and preferably in combination with the further features of the present invention, the production of HTS-readable and interpretable signals, which can be attributed to proximal events associated with receptor binding and/or activation, becomes possible.

As mentioned further above, the methods of the invention preferably use living cells. Preferably, the cells are free, dissociated cells. Preferably, the cells are genetically transformed and/or exhibit heterologous protein expression. In particular, the cells preferably express at least one, two or more recombinant coding nucleotide sequence, which is part of and/or which constitutes the preferred reporting system of the screening methods of the present invention.

Preferably, the cells are immortalized. Preferably, the cells are provided as a cell line expressing at least one heterologous and/or recombinant expression system and/or nucleotide sequence, such as the one or those disclosed herein.

According to an embodiment, the method uses differently transformed and possibly non-transformed cells so as to provide positive and negative control systems.

The present invention provides cells expressing recombinant genes and/or nucleic acids comprising nucleotide sequences encoding chimeric proteins as defined herein. In particular, the cells express recombinant genes and/or nucleotide sequences encoding protein (a) and protein (b) as disclosed independently anywhere in this specification. The present invention also provides cells containing one or both of the chimeric protein (a) and protein (b) as defined herein. In particular, the chimeric protein (a) is a chimeric receptor expressed on the surface of the cell. The cells may be bacterial, plant, fungal, animal and/or human cells, for example.

The two recombinant nucleotide sequences encoding chimeric protein (a) and protein (b), respectively, of the present invention may be introduced in cells by way of a single or separate transfection vectors.

The recombinant nucleotide sequences encoding proteins (a) and/or (b) may be constitutively expressed in their host cells. Alternatively, according to a preferred embodiment, they may be expressed in an inducible and/or repressible manner. Inducible expression is advantageous for the purpose of the present invention, since it enables the experimenter to prepare the cells in accordance with the experimental setting and hypothesis to be tested. Furthermore, inducible expression of the reporting system allows the provision of a negative control, for example in cells where the recombinant nucleotide sequences are not expressed. It is preferably also possible to control and/or determine the amount of protein expressed.

Various control mechanisms are known to induce and/or repress gene transcription and/or expression, including expression of recombinant nucleotide sequences encoding the proteins and/or chimerical proteins of the invention. For example, the TetOn inducible gene expression system is used, in which a nucleotide sequence or gene is under the control of a tet-responsive element (TET). A Tet-On cell line produces the transcription factor rtTA in presence of Doxycycline, thereby making the expression of the nucleotide sequence or gene dependent on the presence of Doxycycline in the culture medium. Of course, other gene expression and/or transcription control systems are available to the skilled person and may be used for the purpose of providing an inducible and/or repressible system controlling the expression of nucleotide sequences that may be part of the invention.

The chimeric protein (a) of the invention comprises a GPCR. As a GPCR, any GPCR of interest can be used. The skilled person can choose a suitable GPCR in dependence of the target of the screening, on the type of activity looked for, the medical purpose of the active agents to be screened and so forth. In the same line, a GPCR from any organism may be selected, depending on the organism in which the active agent is intended to exert an activity. Of course, this specification does not preclude the possibility of using a GPCR isolated of an organism of a given species for screening for agents that have an activity in another species.

GPCRs can be grouped into 6 classes based on sequence homology and functional similarity: Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), Class F (or 6) (Frizzled/Smoothened). In Tables 1-4 below, specific receptors of classes A, B, C and Frizzled, which are found in humans and that can be used for the purpose of the present invention are listed.

According to an embodiment, the GPCR is selected from any one receptor of these classes, in particular from receptors listed in the Tables 1-4 given below, or from groups of such receptors, for example from the receptors of any one of the indicated receptor families. The GPCR may also be selected from homologues of the receptors listed in Tables 1-4 or GPCRs that are not listed in Tables 1-4, for example as found in human or no-human organisms, such as animals, in particular vertebrates, for example mammals, such as, for example rodents, ungulates, non-human primates. The GPCR may be as well found in domestic animals, such as pets (for example, from families Canidae or Felidae) or in livestock. Furthermore, the GPCR may be an artificial and/or a mutated receptor. It is an objective of the present invention to provide a screening method that can be used with different and/or on a variety of receptors. GPCRs constitute a particularly large family of receptors and the principle of the present invention can be applied on any one such receptor.

GPCRs can also be differentiated by the α-subunit of the G protein that is separated from the remainder of the protein (β and γ subunits) during receptor activation. According to an embodiment, the receptor is selected from and/or the cells express a target receptor, which is selected from Gαₛ- and Gα_{i/o}-coupled receptors of all GPCR classes and GPCRs naturally coupled to other G-proteins, for example Gα_{q}, Gα₁₁, Gα₁₂, Gα₁₃, Gα₁₄, Gα₁₅, Gα₁₆, Gα_{T} and Gα_{Z}.

According to an embodiment, the GPCR is selected from any GPCR using any one selected from Gαs, Gαi/o and Gαq for signalling, for example.

According to an embodiment, the GPCR is selected from a human glucagon-like peptide 1 (GLP-1) receptor (GLP-1R), a gastric inhibitory polypeptide receptor (GIPR), an adenosine_{2A} receptor (A_{2A}R) and a muscarinic acetylcholine receptor₄ (M₄R).

According to an embodiment, the GPCR is selected from any one of the group comprising and/or consisting of: a glucagon-like peptide 1 (GLP-1) receptor (GLP-1R), a gastric inhibitory polypeptide receptor (GIPR), an adenosine_{2A} receptor (A_{2A}R), a muscarinic acetylcholine receptor₄ (M₄R), a muscarinic acetylcholine receptor M1 (M1R), a muscarinic acetylcholine receptor M2 (M2R), growth hormone secretagogue receptor (GHSR), a glucagon-like peptide 2 receptor (Glp2R), glucagon receptor (GCGR), a metabotropic glutamate receptor 2 (GluR2), hypocretin (orexin) receptor 1 (O×1R), and orexin receptor type 2 (O×2R).

The Gα subunit of comprised in protein (a) is preferably the one naturally coupling to the selected GPCR. The invention does, however, not exclude the use of a Gα subunit that is not (yet) known or reported to be naturally coupling to the GPCR comprised in the chimeric protein (a), nor the use of a Gα subunit that indeed does not naturally couple to the GPCR selected from protein (a). Accordingly, the Gα subunit in protein (a) may be selected independently from any one of the eight above-listed types of Gα subunits. With respect to the origin of the Gα, Gβ and Gγ subunits, the same as said above with respect to the GPCR applies independently.

The methods of screening of the present invention may be used for the screening of orthosterically and/or allosterically binding active agents. Since the screening methods as disclosed herein are sensitive, dynamic and proximal, also allosterically binding active agents, in particular allosteric modulators can be screened.

According to an embodiment of the screening method of the invention, the cells are preferably exposed to an orthosteric ligand, such as the natural orthosteric ligand, a known agonist or antagonist binding to the binding site of the natural ligand, in the course of the screening procedure. This orthosteric ligand, herein also referred to as “reported orthosteric ligand”, is used to produce a known and/or controlled effect on the target receptor, which is the GPCR present in chimeric protein (a). The reported orthosteric ligand may be added to the sample simultaneously with the candidate agent to be screened (“one-addition” or “co-addition” protocol), or, on the other hand, before or after the addition of a candidate agent (“two-addition protocol”), preferably in an automated manner. In this way, it is possible to also identify allosteric modulators as active agents, which are missed in many screening methods used in the prior art.

The present invention also encompasses the use of a “three-” or another “multiple-addition protocol” in a screening process. A “three-addition” protocol can, for example, include online and/or offline additions of, firstly, the candidate agents, followed by the EC₂₀ of the orthosteric ligand and finally by its EC₈₀ or EC₁₀₀. The third addition might, in another example, comprise the orthosteric antagonist. Other “multiple-addition” protocols (more than three) are also possible, consisting of online additions and/or offline additions.

Preferably, the candidate agent is added before the addition of the reported orthosteric ligand, in the “two-addition protocol”, for example.

Preferably, the reported orthosteric ligand (the control agent) is, independently from the candidate agent, added to all wells of a microtiter plate simultaneously, preferably in an automated manner.

According to an embodiment, the orthosteric ligand producing a known effect is preferably added at a concentration taken from the range of effective concentrations from 5 to 100% (EC₅₋₁₀₀), preferably 5 to 95% (EC₅-EC₉₅) or EC5-EC90 when administered in absence of any other active agent. Preferably, the range is EC₁₀-EC₉₀, more preferably EC₁₅-EC₈₅, most preferably EC₂₀-EC₈₀. The respective EC concentration is preferably pre-determined from exposing the cellular system to increasing concentrations of the orthosteric ligand (concentration-response experiment) using the following formula that models a non-linear regression: Y=Bottom+(Top−Bottom)/(1+10̂((LogEC50−X)*Hill Slope)). The inventors have utilized software for analysis of pharmacological data called GraphPad Prism®, but any such similar software can be used.

A natural ligand is a compound that binds to a receptor under natural conditions in an organism in vivo, and which triggers a corresponding natural cellular response or achieves a corresponding effect. A natural ligand is thus a ligand that is produced by the body of an organism or which is contained in or derived from normal macro- or micronutrients nutrients. A natural ligand preferably binds to the orthosteric site of a receptor, but also allosteric modulators may be natural. The reported orthosteric ligand may thus be a natural ligand of the respective GPCR.

Signal reading may be performed before or after addition of the reported orthosteric ligand, and, independently, before or after addition of the candidate agent to be screened. All these steps may be performed in an automated manner, as described above.

The methods of screening of the present invention may be used for screening libraries of candidate agents. Since a high-throughput and rapid screening is enabled by the methods of the present invention, the HTS methods of the invention are particularly advantageous.

The screening method or assay of the invention, using chimeric polypeptides, is preferably useful for HTS, which can be demonstrated by calculating a “Z′-factor” for the particular assay.

The Z′-factor is a statistical measure indicating the usefulness of an assay for HTS. A score close to 1 indicates that an assay is ideal for HTS and a score less than 0 indicates that an assay is of little use for HTS (see Zhang et al., 1999, J. Biomol. Screen. 4: 67-73). Four parameters needed to calculate the Z-factor are: mean (μ) and standard deviation (σ) of both positive (p) and negative (n) control data (μₚ, σₚ, μₙ, σₙ, respectively). Z′-factor is calculated using the following formula: Z′−factor=1−[3×(σₚ+σₙ)/|μₚ−μₙ|].

According to an embodiment, the Z′-factor of the assay is preferably above 0.0, more preferably equal to or greater than 0.1, 0.2, 0.3, 0.4 and most preferably equal to or greater than 0.5. Z-′factors of specific examples are shown in the Methodology, Results and Z′-Factor of the Screening Experiments section below.

TABLE 1
Class A GPC-Receptors Useful for the Purpose of the Present Invention

| | | | | | | |
|---|---|---|---|---|---|---|
| | Off. | | | | Protein | α- |
| | IUPHAR | Human | HGNC | Nucleotide | Accession | subunit |
| N.l. | name | gene name | ID | Accession number | number | type |

5-Hydroxytryptamine receptors

| | | | | | | |
|---|---|---|---|---|---|---|
| 1 | 5-HT1A | HTR1A | 5286 | NM_000524 | NP_000515 | Gᵢ/G₀ |
| 1 | 5-HT1B | HTR1B | 5287 | NM_000863 | NP_000854 | Gᵢ/G₀ |
| 1 | 5-HT1D | HTR1D | 5289 | NM_000864 | NP_000855 | Gᵢ/G₀ |
| 1 | 5-ht1e | HTR1E | 5291 | NM_000865 | NP_000856 | Gᵢ/G₀ |
| 1 | 5-HT1F | HTR1F | 5292 | NM_000866 | NP_000857 | Gᵢ/G₀ |
| 1 | 5-HT2A | HTR2A | 5293 | NM_000621 | NP_000612 | Gᵢ/G₀ |
| 1 | 5-HT2B | HTR2B | 5294 | NM_000867 | NP_000858 | G_{q}/G₁₁ |
| 1 | 5-HT2C | HTR2C | 5295 | NM_000868 | NP_000859 | G_{q}/G₁₁ |
| 1 | 5-HT4 | HTR4 | 5299 | NM_000870 | NP_000861 | Gₛ |
| 1 | 5-ht5a | HTR5A | 5300 | NM_024012 | NP_076917 | Gᵢ/G₀ |
| 1 | 5-HT6 | HTR6 | 5301 | NM_000871 | NP_000862 | Gₛ |
| 1 | 5-HT7 | HTR7 | 5302 | NM_000872 | NP_000863 | Gₛ |

Acetylcholine receptors (muscarinic)

| | | | | | | |
|---|---|---|---|---|---|---|
| 2 | M1 | CHRM1 | 1950 | NM_000738 | NP_000729 | G_{q}/G₁₁ |
| 2 | M2 | CHRM2 | 1951 | NM_000739 | NP_000730 | Gᵢ/G₀ |
| 2 | M3 | CHRM3 | 1952 | NM_000740 | NP_000731 | G_{q}/G₁₁ |
| 2 | M4 | CHRM4 | 1953 | NM_000741 | NP_000732 | Gᵢ/G₀ |
| 2 | M5 | CHRM5 | 1954 | NM_012125 | NP_036257 | G_{q}/G₁₁ |

Adenosine receptors

| | | | | | | |
|---|---|---|---|---|---|---|
| 3 | A1 | ADORA1 | 262 | NM_000674 | NP_000665 | Gᵢ/G₀ |
| 3 | A2 | ADORA2A | 263 | NM_000675 | NP_000666 | Gₛ |
| 3 | A2B | ADORA2B | 264 | NM_000676 | NP_000667 | Gᵢ/G₀ |
| 3 | A3 | ADORA3 | 268 | NM_000677 | NP_000668 | Gₛ |

Adrenoceptors

| | | | | | | |
|---|---|---|---|---|---|---|
| 4 | α-1A-ac¹⁰⁷⁾ | ADRA1A | 277 | NM_000680 | NP_000671 | G_{q}/G₁₁ |
| 5 | α-1B-ac¹⁰⁷⁾ | ADRA1B | 278 | NM_000679 | NP_000670 | G_{q}/G₁₁ |
| 5 | α-1D-ac¹⁰⁷⁾ | ADRA1D | 280 | NM_000678 | NP_000669 | G_{q}/G₁₁ |
| 5 | α-2A-ac¹⁰⁷⁾ | ADRA2A | 281 | NM_000681 | NP_000672 | Gᵢ/G₀ |
| 5 | α-2B-ac¹⁰⁷⁾ | ADRA2B | 282 | NM_000682 | NP_000673 | Gᵢ/G₀ |
| 5 | α-2C-ac¹⁰⁷⁾ | ADRA2C | 283 | NM_000683 | NP_000674 | Gᵢ/G₀ |
| 4 | β-1-ac¹⁰⁷⁾ | ADRB1 | 285 | NM_000684 | NP_000675 | Gₛ |
| 5 | β-2-ac¹⁰⁷⁾ | ADRB2 | 286 | NM_000024 | NP_000015 | Gₛ |
| 5 | β-3-ac¹⁰⁷⁾ | ADRB3 | 288 | NM_000025 | NP_000016 | Gₛ |

Anaphylatoxin receptors

| | | | | | | |
|---|---|---|---|---|---|---|
| 6 | C3a | C3AR1 | 1319 | NM_004054 | NP_004045 | n/d |
| 7 | C5a | C5R1 | 1338 | NM_001736 | NP_001727 | Gᵢ/G₀ |
| 8 | C5L2 | GPR77 | 4527 | NM_018485 | NP_060955 | n/d |

Angiotensin receptors

| | | | | | | |
|---|---|---|---|---|---|---|
| 9 | AT1 | AGTR1 | 336 | NM_000685 | NP_000676 | Gᵢ/G₀ |
| 9 | AT2 | AGTR2 | 338 | NM_000686 | NP_000677 | Gᵢ/G₀ |

Apelin receptor

| | | | | | | |
|---|---|---|---|---|---|---|
| 10 | APJ | AGTRL1 | 339 | NM_005161 | NP_005152 | Gᵢ/G₀ |

Bile acid receptor

| | | | | | | |
|---|---|---|---|---|---|---|
| 11 | GPBA | GPBAR1 | 19680 | NM_170699 | NP_001070662 | Gₛ |

Bombesin receptors

| | | | | | | |
|---|---|---|---|---|---|---|
| 12 | BB1 | NMBR | 7843 | NM_002511 | NP_002502 | G_{q}/G₁₁ |
| 13 | BB2 | GRPR | 4609 | NM_005314 | NP_005305 | G_{q}/G₁₁ |
| | BB3 | BRS3 | 1113 | NM_001727 | NP_001718 | G_{q}/G₁₁ |

Bradykinin receptors

| | | | | | | |
|---|---|---|---|---|---|---|
| 14 | B1 | BDKRB1 | 1029 | NM_000710 | NP_000701 | Gᵢ/G₀, |
| | | | | | | G_{q}/G₁₁ |
| 14 | B2 | BDKRB2 | 1030 | NM_000623 | NP_000614 | Gₛ, Gᵢ/G₀, |
| | | | | | | G_{q}/G₁₁ |

Cannabinoid receptors

| | | | | | | |
|---|---|---|---|---|---|---|
| 15 | CB1 | CNR1 | 2159 | NM_016083 | NP_057167 | Gᵢ/G₀ |
| 15 | CB2 | CNR2 | 2160 | NM_001841 | NP_001832 | Gᵢ/G₀ |

Chemokine receptors

| | | | | | | |
|---|---|---|---|---|---|---|
| 16 | CCR1 | CCR1 | 1602 | NM_001295 | NP_001286 | Gᵢ/G₀ |
| 17 | CCR2 | CCR2 | 1603 | NM_000647 | NP_000639; | Gᵢ/G₀ |
| | | | | | NP_000638 | |
| 18 | CCR3 | CCR3 | 1604 | NM_001837 | NP_001828 | Gᵢ/G₀ |
| 19 | CCR4 | CCR4 | 1605 | NM_005508 | NP_005499 | Gᵢ/G₀ |
| 20 | CCR5 | CCR5 | 1606 | NM_000579 | NP_000570 | Gᵢ/G₀ |
| 21 | CCR6 | CCR6 | 1607 | NM_004367 | NP_004358 | Gᵢ/G₀ |
| 22 | CCR7 | CCR7 | 1608 | NM_001838 | NP_001829 | Gᵢ/G₀ |
| 23 | CCR8 | CCR8 | 1609 | NM_005201 | NP_005192 | Gᵢ/G₀ |
| 24 | CCR9 | CCR9 | 1610 | NM_006641 | NP_006632 | Gᵢ/G₀ |
| 25 | CCR10 | CCR10 | 4474 | NM_016602 | NP_057686 | Gᵢ/G₀ |
| 26 | CXCR1 | IL8RA | 6026 | NM_000634 | NP_000625 | Gᵢ/G₀ |
| 27 | CXCR2 | IL8RB | 6027 | NM_001557 | NP_001548 | Gᵢ/G₀ |
| 28 | CXCR3 | CXCR3 | 4540 | NM_001504 | NP_001495 | Gᵢ/G₀ |
| 29 | CXCR4 | CXCR4 | 2561 | NM_003467 | NP_003458 | Gᵢ/G₀ |
| 30 | CXCR5 | CXCR5 | 1060 | NM_032966; | NP_116743; | Gᵢ/G₀ |
| | | | | NM_001716 | NP_001707 | |
| 31 | CXCR6 | CXCR6 | 16647 | NM_006564 | NP_006555 | Gᵢ/G₀ |
| 32 | CX3CR1 | CX3CR1 | 2558 | NM_001337 | NP_001328 | Gᵢ/G₀ |
| 33 | XCR1 | XCR1 | 1625 | NM_005283 | NP_005274 | Gᵢ/G₀ |

Cholecystokinin receptors

| | | | | | | |
|---|---|---|---|---|---|---|
| 34 | CCK1 | CCKAR | 1570 | NM_000730 | NP_000721 | G_{q}/G₁₁ |
| 35 | CCK2 | CCKBR | 1571 | NM_176875 | NP_795344 | G_{q}/G₁₁ |

Dopamine receptors

| | | | | | | |
|---|---|---|---|---|---|---|
| 36 | D1 | DRD1 | 3020 | NM_000794 | NP_000785 | Gₛ |
| 36 | D2 | DRD2 | 3023 | NM_000795 | NP_000786 | Gᵢ/G₀ |
| 36 | D3 | DRD3 | 3024 | NM_000796 | NP_000787 | Gᵢ/G₀ |
| 36 | D4 | DRD4 | 3025 | NM_000797 | NP_000788 | Gᵢ/G₀ |
| 36 | D5 | DRD5 | 3026 | NM_000798 | NP_000789 | Gₛ |

Endothelin receptors

| | | | | | | |
|---|---|---|---|---|---|---|
| 37 | ETA | EDNRA | 3179 | NM_001957 | NP_001948 | G_{q}/G₁₁ |
| 38 | ETB | EDNRB | 3180 | NM_000115 | NP_000106 | Gₛ, Gᵢ/G₀, |
| | | | | | | G_{q}/G₁₁ |

Estrogen (G protein coupled) receptor

| | | | | | |
|---|---|---|---|---|---|
| GPER | GPER | 4485 | NM_001505 | NP_001496 | Gᵢ/G₀ |

Formylpeptide receptors

| | | | | | |
|---|---|---|---|---|---|
| FPR1 | FPR1 | 3826 | NM_002029 | NP_002020 | Gᵢ/G₀ |
| FPR2/ALX | FPR2 | 3827 | NM_001462 | NP_001453 | Gᵢ/G₀ |
| FPR3 | FPR3 | 3828 | NM_002030 | NP_002021 | Gᵢ/G₀ |

Free fatty acid receptors

| | | | | | | |
|---|---|---|---|---|---|---|
| 39 | FFA1 | FFAR1 | 4498 | NM_005303 | NP_005294 | G_{q}/G₁₁ |
| | FFA2 | FFAR2 | 4501 | NM_005306 | NP_005297 | G_{q}/G₁₁ |
| | FFA3 | FFAR3 | 4499 | NM_005304 | NP_005395 | Gᵢ/G₀ |
| | GPR42* | GPR42 | 4500 | NM_005305 | NP_005296 | n/d |

Galanin receptors

| | | | | | | |
|---|---|---|---|---|---|---|
| 40 | GAL1 | GALR1 | 4132 | NM_001480 | NP_001471 | n/d |
| 40 | GAL2 | GALR2 | 4133 | NM_003857 | NP_003848 | n/d |
| 40 | GAL3 | GALR3 | 4134 | NM_003614 | NP_003605 | n/d |

Ghrelin receptor

| | | | | | | |
|---|---|---|---|---|---|---|
| 41 | ghrelin | GHSR | 4267 | NM_198407 | NP_940799 | G_{q}/G₁₁ |

Glycoprotein hormone receptors

| | | | | | | |
|---|---|---|---|---|---|---|
| 42 | FSH | FSHR | 3969 | NM_000145 | NP_000136 | Gₛ |
| 43 | LH | LHCGR | 6585 | NM_000233 | NP_000224 | Gₛ |
| 44 | TSH | TSHR | 12373 | NM_000369 | NP_000360 | Gₛ |

Gonadotrophin-releasing hormone receptors

| | | | | | | |
|---|---|---|---|---|---|---|
| 45 | GnRH | GNRHR | 4421 | NM_000406 | NP_000397 | G_{q}/G₁₁ |
| 45 | GnRH2 | GNRHR2 | 16341 | NR_002328 | | n/d |

Histamine receptors

| | | | | | | |
|---|---|---|---|---|---|---|
| 46 | H1 | HRH1 | 5182 | NM_000861 | NP_000852 | G_{q}/G₁₁ |
| 46 | H2 | HRH2 | 5183 | NM_022304 | NP_071640 | G_{q}/G₁₁ |
| 46 | H3 | HRH3 | 5184 | NM_007232 | NP_009163 | Gᵢ/G₀ |
| 46 | H4 | HRH4 | 17383 | NM_021624 | NP_067637 | Gᵢ/G₀ |

KiSS1-derived peptide receptor

| | | | | | |
|---|---|---|---|---|---|
| KiSS1 | KISS1R | 4510 | NM_032551 | NP_115940 | G_{q}/G₁₁ |

Leukotriene receptors

| | | | | | | |
|---|---|---|---|---|---|---|
| 47 | BLT1 | LTB4R | 6713 | NM_181657 | NP_858043 | n/d |
| 47 | BLT2 | LTB4R2 | 19260 | NM_019839 | NP_062813 | n/d |
| 48 | CysLT1 | CYSLTR1 | 17451 | NM_006639 | NP_006630 | n/d |
| 49 | CysLT2 | CYSLTR2 | 18274 | NM_020377 | NP_065110 | n/d |
| 50 | OXE | OXER1 | 24884 | NM_148962 | NP_683765 | n/d |
| | FPR2/ALX | FPR2 | 3827 | NM_001462 | NP_001453 | Gᵢ/G₀ |

Lysophospholipid receptors

| | | | | | | |
|---|---|---|---|---|---|---|
| 51 | LPA1 | LPAR1 | 3166 | NM_001401 | NP_001392 | n/d |
| 51 | LPA2 | LPAR2 | 3168 | NM_004720 | NP_004711 | n/d |
| 51 | LPA3 | LPAR3 | 14298 | NM_012152 | NP_036284 | n/d |
| 52 | S1P1 | S1PR1 | 3165 | NM_001400 | NP_001391 | n/d |
| 52 | S1P2 | S1PR2 | 3169 | NM_004230 | NP_004221 | n/d |
| 52 | S1P3 | S1PR3 | 3167 | NM_005226 | NP_005217 | n/d |
| 52 | S1P4 | S1PR4 | 3170 | NM_003775 | NP_003766 | n/d |
| 52 | S1P5 | S1PR5 | 14299 | NM_030760 | NP_110387 | n/d |

Melanin-concentrating hormone receptors

| | | | | | |
|---|---|---|---|---|---|
| MCH1 | MCHR1 | 4479 | NM_005297 | NP_005288 | Gₛ, Gᵢ/G₀, |
| | | | | | G_{q}/G₁₁ |
| MCH2 | MCHR2 | 20867 | NM_032503; | NP_115892; NP_001035269 | G_{q}/G₁₁ |
| | | | NM_001040179 | | |

Melanocortin receptors

| | | | | | | |
|---|---|---|---|---|---|---|
| 53 | MC1 | MC1R | 6929 | NM_002386 | NP_002377 | Gₛ |
| 54 | MC2 | MC2R | 6930 | NM_000529 | NP_000520 | Gₛ |
| 55 | MC3 | MC3R | 6931 | NM_019888 | NP_063941 | Gₛ |
| 56 | MC4 | MC4R | 6932 | NM_005912 | NP_005903 | Gₛ |
| 53 | MC5 | MC5R | 6933 | NM_005913 | NP_005904 | Gₛ |

Melatonin receptors

| | | | | | | |
|---|---|---|---|---|---|---|
| 57 | MT1 | MTNR1A | 7463 | NM_005958 | NP_005949 | Gᵢ/G₀ |
| 57 | MT2 | MTNR1B | 7464 | NM_005959 | NP_005950 | Gᵢ/G₀ |

Motilin receptor

| | | | | | | |
|---|---|---|---|---|---|---|
| 58 | motilin | MLNR | 4495 | NM_001507 | NP_001498 | G_{q}/G₁₁, |
| | | | | | | G₁₂/G₁₃ |

Neuromedin U receptors

| | | | | | | |
|---|---|---|---|---|---|---|
| 59 | NMU1 | NMUR1 | 4518 | NM_006056 | NP_006047 | G_{q}/G₁₁ |
| 59 | NMU2 | NMUR2 | 16454 | NM_020167 | NP_064552 | n/d |

Neuropeptide FF/neuropeptide AF receptors

| | | | | | |
|---|---|---|---|---|---|
| NPFF1 | NPFFR1 | 17425 | NM_022146 | NP_071429 | Gᵢ/G₀ |
| NPFF2 | NPFFR2 | 4525 | | | Gᵢ/G₀ |

Neuropeptide S receptor

| | | | | | |
|---|---|---|---|---|---|
| NPS | NPSR1 | 23631 | NM_207172 | NP_997055 | G_{q}/G₁₁ |

Neuropeptide FF/neuropeptide B receptors

| | | | | | |
|---|---|---|---|---|---|
| NPBW1 | NPBWR1 | 4522 | NM_005285 | NP_005276 | Gᵢ/G₀ |
| NPBW2 | NPBWR2 | 4530 | NM_005286 | NP_005277 | Gᵢ/G₀ |

Neuropeptide Y receptors

| | | | | | | |
|---|---|---|---|---|---|---|
| 60 | Y1 | NPY1R | 7956 | NM_000909 | NP_000900 | Gᵢ/G₀ |
| 60 | Y2 | NPY2R | 7957 | NM_000910 | NP_000901 | Gᵢ/G₀ |
| 61 | Y4 | PPYR1 | 9329 | NM_005972 | NP_005963 | Gᵢ/G₀ |
| 60 | Y5 | NPY5R | 7958 | NM_006174 | NP_006165 | Gᵢ/G₀ |

Neurotensin receptors

| | | | | | | |
|---|---|---|---|---|---|---|
| 62 | NTS1 | NTSR1 | 8039 | NM_002531 | NP_002522 | G_{q}/G₁₁ |
| 62 | NTS2 | NTSR2 | 8040 | NM_012344 | NP_036476 | G_{q}/G₁₁ |

Nicotinic acid receptor family

| | | | | | | |
|---|---|---|---|---|---|---|
| 63 | GPR81¹⁰⁸⁾ | GPR81 | 4532 | NM_032554 | NP_115943 | Gᵢ/G₀ |
| 64 | GPR109A¹⁰⁸ | GPR109A | 24827 | NM_177551 | NP_808219 | Gᵢ/G₀ |
| 65 | GPR109B¹⁰⁸ | GPR109B | 16824 | NM_006018 | NP_006009 | Gᵢ/G₀ |

Opioid receptors

| | | | | | | |
|---|---|---|---|---|---|---|
| 66 | δ | OPRD1 | 8153 | NM_000911 | NP_000902 | Gᵢ/G₀ |
| 67 | kappa | OPRK1 | 8154 | NM_000912 | NP_000903 | Gᵢ/G₀ |
| 68 | mu | OPRM1 | 8156 | NM_000914 | NP_000905 | Gᵢ/G₀ |
| 69 | NOP | OPRL1 | 8155 | NM_000913 | NP_000904 | Gᵢ/G₀ |

Orextn receptors

| | | | | | | |
|---|---|---|---|---|---|---|
| 70 | OX1 | HCRTR1 | 4848 | NM_001525 | NP_001516 | Gᵢ/G₀ |
| 70 | OX2 | HCRTR2 | 4849 | NM_001526 | NP_001517 | G_{q}/G₁₁ |

P2Y receptors

| | | | | | | |
|---|---|---|---|---|---|---|
| 71 | P2Y1 | P2RY1 | 8539 | NM_002563 | NP_002554 | G_{q}/G₁₁ |
| 72, | P2Y2 | P2RY2 | 8541 | NM_002564 | NP_002555 | G_{q}/G₁₁ |
| 73 | | | | | | |
| 72 | P2Y4 | P2RY4 | 8542 | NM_002565 | NP_002556 | G_{q}/G₁₁ |
| 74 | P2Y6 | P2RY6 | 8543 | NM_004154 | NP_004145 | G_{q}/G₁₁ |
| 73 | P2Y11 | P2RY11 | 8540 | NM_002566 | NP_002557 | G_{q}/G₁₁ |
| 71 | P2Y12 | P2RY12 | 18124 | NM_022788 | NP_073625 | Gᵢ/G₀ |
| 71 | P2Y13 | P2RY13 | 4537 | NM_176894; | NP_795713; NP_076403 | Gᵢ/G₀ |
| | | | | NM_023914 | | |
| 75 | P2Y14 | P2RY14 | 16442 | NM_014879 | NP_055694 | Gᵢ/G₀ |

Peptide P518 receptor

| | | | | | | |
|---|---|---|---|---|---|---|
| 76 | QRFP | QRFPR | 15565 | NM_198179 | NP_937822 | n/d |

Platelet-activating factor receptor

| | | | | | | |
|---|---|---|---|---|---|---|
| 77 | PAF | PTAFR | 9582 | NM_000952 | NP_000943 | G_{q}/G₁₁ |

Prokineticin receptors

| | | | | | |
|---|---|---|---|---|---|
| PKR1 | PROKR1 | 4524 | NM_138964 | NP_620414 | G_{q}/G₁₁ |
| PKR2 | PROKR2 | 15836 | NM_144773 | NP_658986 | G_{q}/G₁₁, |
| | | | | | Gᵢ/G₀ |

Prolactin-releasing peptide receptor

| | | | | | |
|---|---|---|---|---|---|
| PRRP | PRLHR | 4464 | NM_004248 | NP_004239 | n/d |

Prostanoid receptors

| | | | | | | |
|---|---|---|---|---|---|---|
| 78 | DP1 | PTGDR | 9591 | NM_000953 | NP_000944 | Gₛ |
| 79 | DP2 | GPR44 | 4502 | NM_004778 | NP_004769 | Gᵢ/G₀ |
| 80 | EP1 | PTGER1 | 9593 | NM_000955 | NP_000946 | G_{q}/G₁₁ |
| 80 | EP2 | PTGER2 | 9594 | NM_000956 | NP_000947 | Gₛ |
| 80 | EP3 | PTGER3 | 9595 | NM_000957 | NP_000948 | Gᵢ/G₀ |
| 80 | EP4 | PTGER4 | 9596 | NM_000958 | NP_000949 | Gₛ |
| 81 | FP | PTGFR | 9600 | NM_000959 | NP_000950 | G_{q}/G₁₁ |
| 82 | IP1 | PTGIR | 9602 | NM_000960 | NP_000951 | Gₛ |
| 83 | TP | TBXA2R | 11608 | NM_001060 | NP_001051 | G_{q}/G₁₁ |

Protease-activated receptors

| | | | | | | |
|---|---|---|---|---|---|---|
| 84 | PAR1 | F2R | 3537 | NM_001992 | NP_001983 | n/d |
| 85 | PAR2 | F2RL1 | 3538 | NM_005242 | NP_005233 | n/d |
| 84 | PAR3 | F2RL2 | 3539 | NM_004101 | NP_004092 | n/d |
| 85 | PAR4 | F2RL3 | 3540 | NM_003950 | NP_003941 | n/d |

Relaxin family peptide receptors

| | | | | | |
|---|---|---|---|---|---|
| RXFP1 | RXFP1 | 19718 | NM_021634 | NP_067647 | Gₛ, Gᵢ/G₀ |
| RXFP2 | RXFP2 | 17318 | NM_130806 | NP_570718 | Gₛ, Gᵢ/G₀ |
| RXFP3 | RXFP3 | 24883 | NM_016568 | NP_057652 | Gᵢ/G₀ |
| RXFP4 | RXFP4 | 14666 | NM_181885 | NP_871001 | Gᵢ/G₀ |

Somatostatin receptors

| | | | | | | |
|---|---|---|---|---|---|---|
| 86 | sst1 | SSTR1 | 11330 | NM_001049 | NP_001040 | Gᵢ/G₀ |
| 86 | sst2 | SSTR2 | 11331 | NM_001050 | NP_001041 | Gᵢ/G₀ |
| 86 | sst3 | SSTR3 | 11332 | NM_001051 | NP_001042 | Gᵢ/G₀ |
| 86 | sst4 | SSTR4 | 11333 | NM_001052 | NP_001043 | Gᵢ/G₀ |
| 86 | sst5 | SSTR5 | 11334 | NM_001053 | NP_001044 | Gᵢ/G₀ |

Tachykinin receptors

| | | | | | | |
|---|---|---|---|---|---|---|
| 87 | NK1 | TACR1 | 11526 | NM_001058 | NP_001049 | Gₛ, |
| | | | | | | G_{q}/G₁₁ |
| 88 | NK2 | TACR2 | 11527 | NM_001057 | NP_001048 | Gₛ, |
| | | | | | | G_{q}/G₁₁ |
| 89 | NK3 | TACR3 | 11528 | NM_001059 | NP_001050 | G_{q}/G₁₁ |

Thyrotropin-releasing hormone receptor

| | | | | | | |
|---|---|---|---|---|---|---|
| 90 | TRH1 | TRHR | 12299 | NM_003301 | NP_003292 | G_{q}/G₁₁ |

Trace amine receptor

| | | | | | |
|---|---|---|---|---|---|
| TA1 | TAAR1 | 17734 | NM_138327 | NP_612200 | Gₛ |

Urotensin receptor

| | | | | | | |
|---|---|---|---|---|---|---|
| 91 | UT | UTS2R | 4468 | NM_018949 | NP_061822 | G_{q}/G₁₁ |

Vasopressin and oxytocin receptors

| | | | | | | |
|---|---|---|---|---|---|---|
| 92 | V1A | AVPR1A | 895 | NM_000706 | NP_000697 | G_{q}/G₁₁ |
| 92 | V1B | AVPR1B | 896 | NM_000707 | NP_000698 | G_{q}/G₁₁ |
| 92 | V2 | AVPR2 | 897 | NM_000054 | NP_000045 | Gₛ |
| 93 | OT | OXTR | 8529 | NM_000916 | NP_000907 | G_{q}/G₁₁ |

Class A Orphans

| | | | | | | |
|---|---|---|---|---|---|---|
| | CCRL2 | CCRL2 | 1612 | NM_003965 | NP_003956 | n/d |
| 94 | CMKLR1 | CMKLR1 | 2121 | NM_004072 | NP_004063 | n/d |
| | CMKOR1 | CMKOR1 | 23692 | NM_020311 | NP_064707 | n/d |
| | EBI2 | GPR183 | 3128 | NM_004951 | NP_004942 | n/d |
| | GPR42* | GPR42 | 4500 | NM_005305 | NP_005296 | n/d |
| | GPR1 | GPR1 | 4463 | NM_005279 | NP_005270 | n/d |
| 52 | GPR3 | GPR3 | 4484 | NM_005281 | NP_005272 | n/d |
| 95 | GPR4 | GPR4 | 4497 | NM_005282 | NP_005273 | n/d |
| 52 | GPR6 | GPR6 | 4515 | NM_005284 | NP_005275 | n/d |
| 52 | GPR12 | GPR12 | 4466 | NM_005288 | NP_005279 | n/d |
| | GPR15 | GPR15 | 4469 | NM_005290 | NP_005281 | n/d |
| 96 | GPR17 | GPR17 | 4471 | NM_005291 | NP_005282 | n/d |
| | GPR18 | GPR18 | 4472 | NM_005292 | NP_005283 | n/d |
| | GPR19 | GPR19 | 4473 | NM_006143 | NP_006134 | n/d |
| | GPR20 | GPR20 | 4475 | NM_005293 | NP_005284 | n/d |
| | GPR21 | GPR21 | 4476 | NM_005294 | NP_005285 | n/d |
| | GPR22 | GPR22 | 4477 | NM_005295 | NP_005286 | n/d |
| 51 | GPR23 | GPR23 | 4478 | NM_005296 | NP_005287 | n/d |
| | GPR25 | GPR25 | 4480 | NM_005298 | NP_005289 | n/d |
| | GPR26 | GPR26 | 4481 | NM_153442 | NP_703143 | n/d |
| | GPR27 | GPR27 | 4482 | NM_018971 | NP_061844 | n/d |
| | GPR31 | GPR31 | 4486 | NM_005299 | NP_005290 | n/d |
| | GPR32 | GPR32 | 4487 | NM_001506 | NP_001497 | n/d |
| | GPR33** | GPR33 | 4489 | NG_001132 | | n/d |
| | GPR34 | GPR34 | 4490 | NM_005300 | NP_005291 | n/d |
| 97 | GPR35 | GPR35 | 4492 | NM_005301 | NP_005292 | n/d |
| 98 | GPR37 | GPR37 | 4494 | NM_005302 | NP_005293 | n/d |
| | GPR37L1 | GPR37L1 | 14923 | NM_004767 | NP_004758 | n/d |
| 99 | GPR39 | GPR39 | 4496 | NM_001508 | NP_001499 | n/d |
| | GPR45 | GPR45 | 4503 | NM_007227 | NP_009158 | n/d |
| | GPR50 | GPR50 | 4506 | NM_004224 | NP_004215 | n/d |
| | GPR52 | GPR52 | 4508 | NM_005684 | NP_005675 | n/d |
| 100 | GPR55 | GPR55 | 4511 | NM_005683 | NP_005674 | n/d |
| | GPR61 | GPR61 | 13300 | NM_031936 | NP_144142 | n/d |
| | GPR62 | GPR62 | 13301 | NM_080865 | NP_543141 | n/d |
| | GPR63 | GPR63 | 13302 | NM_030784 | NP_110411 | n/d |
| 95 | GPR65 | GPR65 | 4517 | NM_003608 | NP_003599 | n/d |
| 95 | GPR68 | GPR68 | 4519 | NM_003485 | NP_003476 | n/d |
| 101 | GPR75 | GPR75 | 4526 | NM_006794 | NP_006785 | n/d |
| | GPR78 | GPR78 | 4528 | NM_080819 | NP_543009 | n/d |
| | GPR79* | GPR79 | 4529 | NG_001272 | | n/d |
| | GPR82 | GPR82 | 4533 | NM_080817 | NP_543007 | n/d |
| | GPR83 | GPR83 | 4523 | NM_016540 | NP_057624 | n/d |
| | GPR84 | GPR84 | 4535 | NM_020370 | NP_065103 | n/d |
| | GPR85 | GPR85 | 4536 | NM_018970 | NP_061842 | n/d |
| | GPR87 | GPR87 | 4538 | NM_023915 | NP_076404 | n/d |
| | GPR88 | GPR88 | 4539 | NM_022049 | NP_071332 | n/d |
| 51 | GPR92 | GPR92 | 13307 | NM_020400 | NP_065133 | n/d |
| | GPR101 | GPR101 | 14963 | NM_054021 | NP_473362 | n/d |
| 102 | GPR119 | GPR119 | 19060 | NM_178471 | NP_848566 | n/d |
| 103 | GPR120 | GPR120 | 19061 | NM_181745 | NP_859529 | n/d |
| 95 | GPR132 | GPR132 | 17482 | NM_013345 | NP_037477 | n/d |
| | GPR135 | GPR135 | 19991 | NM_022571 | NP_072093 | n/d |
| | GPR139 | GPR139 | 19995 | NM_001002911 | NP_001002911 | n/d |
| | GPR141 | GPR141 | 19997 | NM_181791 | NP_861456 | n/d |
| | GPR142 | GPR142 | 20088 | NM_181790 | NP_861455 | n/d |
| | GPR146 | GPR146 | 21718 | NM_138445 | NP_612454 | n/d |
| | GPR148 | GPR148 | 23623 | NM_207364 | NP_997247 | n/d |
| | GPR149 | GPR149 | 23627 | NM_001038705 | NP_001033794 | n/d |
| | GPR150 | GPR150 | 23628 | NM_954713 | NP_954713 | n/d |
| | GPR151 | GPR151 | 23624 | NM_194251 | NP_919227 | n/d |
| | GPR152 | GPR152 | 23622 | NM_206997 | NP_996880 | n/d |
| | GPR153 | GPR153 | 23618 | NM_207370 | NP_997253 | n/d |
| | GPR160 | GPR160 | 23693 | NM_014373 | NP_055188 | n/d |
| | GPR161 | GPR161 | 23694 | NM_153832 | NP_722561 | n/d |
| | GPR162 | GPR162 | 16693 | NM_019858 | NP_062832 | n/d |
| | GPR171 | GPR171 | 30057 | NM_013308 | NP_013308 | n/d |
| | GPR173 | GPR173 | 18186 | NM_018969 | NP_061842 | n/d |
| | GPR174 | GPR174 | 30245 | NM_032553 | NP_115942 | n/d |
| | GPR182 | GPR182 | 13708 | NM_007264 | NP_009195 | n/d |
| | LGR4 | LGR4 | 13299 | NM_018490 | NP_060960 | n/d |
| | LGR5 | LGR5 | 4504 | NM_003667 | NP_003658 | n/d |
| | LGR6 | LGR6 | 19719 | NM_021636 | NP_067649 | n/d |
| | MAS1 | MAS1 | 6899 | NM_002377 | NP_002377 | n/d |
| | MAS1L | MAS1L | 13961 | NM_052967 | NP_443199 | n/d |
| 104 | MRGPRD | MRGPRD | 29626 | NM_198923 | NP_944605 | n/d |
| | MRGPRE | MRGPRE | 30694 | NM_001039165 | NP_001034254 | n/d |
| | MRGPRF | MRGPRF | 24828 | NM_145015 | NP_659452 | n/d |
| | MRGPRG | MRGPRG | 24829 | XM_001126608 | XP_001126608 | n/d |
| | MRGPRX1 | MRGPRX1 | 17962 | NM_147199 | NP_671732 | n/d |
| | MRGPRX2 | MRGPRX2 | 17983 | NM_054030 | NP_054030 | n/d |
| | MRGPRX3 | MRGPRX3 | 17980 | NM_054031 | NP_473372 | n/d |
| | MRGPRX4 | MRGPRX4 | 17617 | NM_054032 | NP_473373 | n/d |
| | OPN3 | OPN3 | 14007 | NM_014322 | NP_055137 | n/d |
| | OPN5 | OPN5 | 19992 | NM_181744 | NP_859528 | n/d |
| 105 | OXGR1 | OXGR1 | 4531 | NM_080818 | NP_543008 | n/d |
| | P2RY5 | P2RY5 | 15520 | NM_005767 | NP_005758 | n/d |
| | P2RY8 | P2RY8 | 15524 | NM_178129 | NP_835230 | n/d |
| | P2RY10 | P2RY10 | 19906 | NM_014499 | NP_055314 | n/d |
| 106 | SUCNR1 | SUCNR1 | 4542 | NM_033050 | NP_149039 | n/d |
| | TAAR2 | TAAR2 | 4514 | NM_001033080 | NP_00128252 | n/d |
| | TAAR3* | TAAR3 | 4513 | NG_002481 | | n/d |
| | TAAR4* | | | | | n/d |
| | TAAR5 | TAAR5 | 30236 | NM_003967 | NP_003958 | n/d |
| | TAAR6 | TAAR6 | 20978 | NM_175067 | NP_778237 | n/d |
| | TAAR8 | TAAR8 | 14964 | NM_053278 | NP_444508 | n/d |
| | TAAR9 | TAAR9 | 20977 | NM_175057 | NP_778227 | n/d |

Non-signalling 7TM chemokine-binding proteins

| | | | | | |
|---|---|---|---|---|---|
| CCPB2 | CCBP2 | 1565 | NM_001296 | NP_001287 | n/d |
| CCRL1 | CCRL1 | 1611 | NM_178445 | NP_848540 | n/d |
| FY | DARC | 4035 | NM_002036 | NP_002027 | n/d |

Legend Table 1:
N.l.: Natural ligands: 1) 5-hydroxytryptamine; 2) acetylcholine; 3) adenosine; 4) noradrenaline; 5) adrenaline; 6) anaphylatoxin C3a; 7) anaphylatoxin C5a; 8) anaphylatoxin C5a, C5a des Arg74; 9) angiotensin; 10) apelin; 11) bile acids; 12) neuromedin B; 13) gastrin-releasing peptide; 14 bradykinin; 15) cannabinoid; 16) CCL3, CCL5, CCL7, CCL8, CCL13-16, CCL23; 17) CCL2, CCL7, CCL8, CCL13; 18) CCL11; CCL5, CCL7, CCL8, CCL13, CCL15, CCL24, CCL26; 19) CCL17, CCL22; 20) CCL3, CCL4, CCL5, CCL8, CCL14; 21) CCL20; 22) CCL19, CCL21; 23) CCL1, CCL4, CCL17; 24) CCL25; 25) CCL26-28; 26) CXCL8; 27) CXCL1-3, CXCL5-8, macrophage derived lectin; 28) CXCL9-11; 29) CXCL12; 30) CXCL13; 31) CXCL16; 32) CX3CL1; 33) XCL1, XCL2; 34) cholecystokinin; 35) cholecystokinin, gastrin; 36) dopamine; 37) endothelin 1 and 2; 38) endothelins 1, 2 and 3; 39) long chain carboxylic acids; 40) galanin; 41) ghrelin; 42) follicle-stimulating hormone; 43) luteinizing hormone, chorionic gonadotropin; 44) thyroid-stimulating hormone; 45) gonadotrophin-releasing hormone; 46) histamine; 47) leukotriene B4; 48) leukotriene D4; 49) leukotriene C4; 50) 5-oxo-ETE; 51) lysophosphatidic acid; 52) sphingosine-1-phosphate; 53) α-melanocyte stimulating hormone; 54) adrenocorticotropic hormone; 55) γ-melanocyte stimulating hormone; 56) β- melanocyte stimulating hormone; 57) melatonin; 58) motilin; 59) neuromedin U; 60) neuropeptide Y; 61) pancreatic polypeptide; 62) neurotensin; 63) lactate; 64) nicotinic acid (high affinity); 65) nicotinic acid (low affinity); 66) β-endorphin; 67) dynorphin A; 68) β-endorphin; 69) nociceptin/orphanin FQ; 70) orexin A, orexin B; 71) ADP; 72) UTP; 73) ATP; 74) UDP; 75) UDP-glucose; 76) RF-amide P518 gene product; 77) platelet-activating factor; 78) prostaglandin D2; 79) 11-dehydro-thromboxane B2; 80) prostaglandin E2; 81) prostaglandin F2a; 82) prostacyclin; 83) thromboxane A2; 84) thrombin; 85) serine proteases; 86) somatostatin; 87) substance P; 88) neurokinin A; 89) neurokinin B; 90) thyrotropin-releasing hormone; 91) urotensin II; 92) vasopressin; 93) oxytocin; 94) RARRES2, resolvin E1, TIG2; 95) (lyso)phospholipid mediators; 96) cysteinyl-leukotrienes (CysLTs), uracil nucleotides; 97) kynurenic acid; 98) neuropeptide head activator; 99) obestatin; 100) lysophosphatidyl-inositol; 101) RANTES; 102) oleoylethanolamide; 103) free fatty acids; 104) β-alanine; 105) α-ketoglutarate; 106) succinate.
Others: 107) ac = adrenoceptor; 108) temporary name;
*probable pseudogene;
**pseudogene in most individuals;
n/d = not determined.

TABLE 2
Class B GPC-Receptors Useful for the Purpose of the Present Invention

| | | | | | | |
|---|---|---|---|---|---|---|
| | | | | | | α- |
| | IUPHAR | | HGNC | Nucleotide | Protein | subunit |
| N.l. | name | Gene name | ID | Accession no. | Accession no. | type |

Calcitonin receptors

| | | | | | | |
|---|---|---|---|---|---|---|
| 109 | CT¹¹⁷⁾ | CALCR | 1440 | NM_001742 | NP_001733 | Gₛ |
| 110 | CALCRL¹¹⁸⁾ | CALCRL | 16709 | NM_005795 | NP_005786 | n/d |

Corticotropin-releasing factor receptors

| | | | | | |
|---|---|---|---|---|---|
| CRF1 | CRHR1 | 2357 | NM_004382 | NP_004373 | Gₛ |
| CRF2 | CRHR2 | 2358 | NM_001883 | NP_001874 | Gₛ |

Glucagon receptor family

| | | | | | | |
|---|---|---|---|---|---|---|
| | GHRH | GHRHR | 4266 | NM_000823 | NP_000814 | Gₛ |
| | GIP | GIPR | 4271 | NM_000164 | NP_000155 | Gₛ |
| | GLP-1 | GLP1R | 4324 | NM_002062 | NP_002053 | Gₛ |
| | GLP-2 | GLP2R | 4325 | NM_004246 | NP_004237 | n/a |
| 111 | glucagon | GCGR | 4192 | NM_000160 | NP_000151 | Gₛ |
| 112 | secretin | SCTR | 10608 | NM_002980 | NP_002971 | n/a |

Parathyroid hormone receptors

| | | | | | | |
|---|---|---|---|---|---|---|
| 113 | PTH1 | PTH1R | 9608 | NM_000316 | NP_000307 | Gₛ |
| 114 | PTH2 | PTHR2 | 9609 | NM_005048 | NP_005039 | n/d |

VIP and PACAP receptors

| | | | | | | |
|---|---|---|---|---|---|---|
| 115 | PAC1 | ADCYAP1R1 | 242 | NM_001118 | NP_001109 | Gₛ |
| 116 | VPAC1 | VIPR1 | 12694 | NM_004624 | NP_004615 | Gₛ |
| 116 | VPAC2 | VIPR2 | 12695 | NM_003382 | NP_003373 | Gₛ |

Class B Orphans

| | | | | | |
|---|---|---|---|---|---|
| BAI1 | BAI1 | 943 | NM_001702 | NP_001693 | n/d |
| BAI2 | BAI2 | 944 | NM_001703 | NP_001694 | n/d |
| BAI3 | BAI3 | 945 | NM_001704 | NP_001695 | n/d |
| CD97 | CD97 | 1711 | NM_078481 | NP_510966 | n/d |
| CELSR1 | CELSR1 | 1850 | NM_014246 | NP_005061 | n/d |
| CELSR2 | CELSR2 | 3231 | NM_001408 | NP_001399 | n/d |
| CELSR3 | CELSR3 | 3230 | NM_001407 | NP_001398 | n/d |
| ELTD1 | ELTD1 | 20822 | NM_022159 | NP_071442 | n/d |
| EMR1 | EMR1 | 3336 | NM_001974 | NP_001965 | n/d |
| EMR2 | EMR2 | 3337 | NM_013447 | NP_038475 | n/d |
| EMR3 | EMR3 | 23647 | NM_032571 | NM_032571 | n/d |
| EMR4* | EMR4 | 19240 | | | n/d |
| GPR56 | GPR56 | 4512 | NM_005682 | NP_005673 | n/d |
| GPR64 | GPR64 | 4516 | NM_001079858 | NP_001073328 | n/d |
| GPR97 | GPR97 | 13728 | NM_170776 | NP_740746 | n/d |
| GPR98 | GPR98 | 17416 | NM_032119 | NP_115495 | n/d |
| GPR110 | GPR110 | 18990 | NM_153840 | NP_722582 | n/d |
| GPR111 | GPR111 | 18991 | NM_153839 | NP_722581 | n/d |
| GPR112 | GPR112 | 18992 | NM_153834 | NP_722576 | n/d |
| GPR113 | GPR113 | 18989 | NM_153835 | NP_722577 | n/d |
| GPR114 | GPR114 | 19010 | NM_153837 | NP_722579 | n/d |
| GPR115 | GPR115 | 19011 | NM_153838 | NP_722580 | n/d |
| GPR116 | GPR116 | 19030 | NM_015234 | NP_056049 | n/d |
| GPR123 | GPR123 | 13838 | NM_001083909 | NP_001077378 | n/d |
| GPR124 | GPR124 | 17849 | NM_032777 | NP_116166 | n/d |
| GPR125 | GPR125 | 13839 | NM_145290 | NP_660333 | n/d |
| GPR126 | GPR126 | 13841 | NM_020455 | NP_065188 | n/d |
| GPR128 | GPR128 | 19241 | NM_032787 | NP_116176 | n/d |
| GPR133 | GPR133 | 19893 | NM_198827 | NP_942122 | n/d |
| GPR143 | GPR143 | 20145 | NM_000273 | NP_000264 | n/d |
| GPR144 | GPR144 | 18651 | | | n/d |
| GPR157 | GPR157 | 23687 | NM_024980 | NP_079256 | n/d |
| LPHN1 | LPHN1 | 20973 | NM_001008701; | NP_001008701; | n/d |
| | | | NM_014921 | NP_055736 | |
| LPHN2 | LPHN2 | 18582 | NM_012302 | NP_036434 | n/d |
| LPHN3 | LPHN3 | 20974 | NM_015236 | NP_056051 | n/d |

Legend Table 2:
N.l.: Natural ligands; 109) amylin, CGRP; 110) adrenomedullin, CGRP; 111) glucagon; 112) secretin; 113) parathyroid hormone; 114) TIP-39; 115) PACAP; 116) VIP, PACAP; 117) CALCR can dimerize to form the calcitonin R. However, if it associates with RAMP-1, -2, or -3, it becomes Amylin 1, 2, or 3 receptor. 118) CALCRL needs to associate to RAMP1 to form the CGRP receptor, and with RAMP2 or 3 to form Adrenomedulin 1 or 2 receptor.

TABLE 3
Class C GPC-Receptors Useful for the Purpose of the Present Invention

| | | | | | | |
|---|---|---|---|---|---|---|
| | Official | Human | | Human | Human | a- |
| | IUPHAR | gene | HGNC | nucleotide | protein | subunit |
| Ligand | name | name | ID | RefSeq | RefSeq | type |

Calcium-sensing receptors

| | | | | | | |
|---|---|---|---|---|---|---|
| 119 | CaS | CASR | 1514 | NM_000388 | NP_00379 | Gᵢ/G₀, |
| | | | | | | G_{q}/G₁₁, |
| | | | | | | G₁₂/G₁₃ |
| | GPRC6 | GPRC6A | 18510 | NM_148963 | NP_683766 | G_{q}/G₁₁ |

GABA B receptors

| | | | | | | |
|---|---|---|---|---|---|---|
| 120 | GABAB1¹²²⁾ | GABBR1 | 4070 | NM_001470 | NP_001461 | n/d |
| | GABAB2 | GABBR2 | 4507 | NM_005458 | NP_005449 | n/d |

GPRC5 receptors

| | | | | | |
|---|---|---|---|---|---|
| RAIG1 | GPRC5A | 9836 | NM_003979 | NP_003970 | n/d |
| RAIG2 | GPRC5B | 13308 | NM_016235 | NP_057319 | n/d |
| RAIG3 | GPRC5C | 13309 | NM_018653 | NP_061123 | n/d |
| RAIG4 | GPRC5D | 13310 | NM_018654 | NP_061124 | n/d |

Metabotropic glutamate receptors

| | | | | | | |
|---|---|---|---|---|---|---|
| 121 | mGlu1 | GRM1 | 4593 | NM_000838 | NP_000829 | G_{q}/G₁₁ |
| 121 | mGlu2 | GRM2 | 4594 | NM_000839 | NP_000830 | Gᵢ/G₀ |
| 121 | mGlu3 | GRM3 | 4595 | NM_000840 | NP_000831 | Gᵢ/G₀ |
| 121 | mGlu4 | GRM4 | 4596 | NM_000841 | NP_000832 | Gᵢ/G₀ |
| 121 | mGlu5 | GRM5 | 4597 | NM_000842 | NP_000833 | G_{q}/G₁₁ |
| 121 | mGlu6 | GRM6 | 4598 | NM_000843 | NP_000834 | Gᵢ/G₀ |
| 121 | mGlu7 | GRM7 | 4599 | NM_000844 | NP_000835 | Gᵢ/G₀ |
| 121 | mGlu8 | GRM8 | 4600 | NM_000845 | NP_000836 | Gᵢ/G₀ |

Class C Orphans

| | | | | | |
|---|---|---|---|---|---|
| GPR156 | GPR156 | 20844 | NM_153002 | NP_694547 | n/a |
| GPR158 | GPR158 | 23689 | NM_020752 | NP_065803 | n/a |
| GPR179 | GPR179 | 31371 | NM_001004334 | NP_001004334 | n/a |
| RAIG1 | GPRC5A | 9836 | NM_003979 | NP_003970 | n/a |
| RAIG2 | GPRC5B | 13308 | NM_016235 | NP_057319 | n/a |
| RAIG3 | GPRC5C | 13309 | NM_018653 | NP_061123 | n/a |
| RAIG4 | GPRC5D | 13310 | NM_018654 | NP_061124 | n/a |

Legend Table 3:
N.l.: Natural ligands; 119) Calcium; 120) GABA; 121) Glutamate; 122) GABAB1 dimerizes with GABAB2 to form the GABAB receptor that is Gi/Go coupled.

TABLE 4
Class “Frizzled” GPC-Receptors for the Purpose of the Present Invention

| | | | | | | |
|---|---|---|---|---|---|---|
| | | | | Nucleotide | Protein | |
| Natural | IUPHAR | Gene | | Accession | Accession | |
| ligand | name | name | HGNC ID | number | number | G-protein |

Frizzled receptors

| | | | | | | |
|---|---|---|---|---|---|---|
| Wnt3A, | FZD1 | FZD1 | 4038 | NM_003505 | NP_003496 | Gᵢ/G₀, |
| Wnt3, | | | | | | G_{q}/G₁₁ |
| Wnt1, | | | | | | |
| Wnt2 | | | | | | |
| Wnt | FZD2 | FZD2 | 4040 | NM_001466 | NP_001457 | Gᵢ/G₀ |
| Wnt | FZD3 | FZD3 | 4041 | NM_017412 | NP_059108 | n/a |
| Wnt | FZD4 | FZD4 | 4042 | NM_012193 | NP_036325 | n/a |
| Wnt | FZD5 | FZD5 | 4043 | NM_003468 | NP_003459 | n/a |
| Wnt | FZD6 | FZD6 | 4044 | NM_003506 | NP_003497 | n/a |
| Wnt | FZD7 | FZD7 | 4045 | NM_003507 | NP_003498 | n/a |
| Wnt | FZD8 | FZD8 | 4046 | NM_031866 | NP_114072 | n/a |
| Wnt | FZD9 | FZD9 | 4047 | NM_003508 | NP_003499 | n/a |
| Wnt | FZD10 | FZD10 | 4039 | NM_007197 | NP_009128 | n/a |
| constitutive | SMO | SMO | 11119 | NM_005631 | NP_005622 | Gᵢ/G₀, |
| | | | | | | G₁₂/G₁₃ |

The present invention will now be illustrated by way of examples. These examples do not limit the scope of this invention, which is defined by the appended claims.

### EXAMPLES

### Molecular Biology of Bret Systems

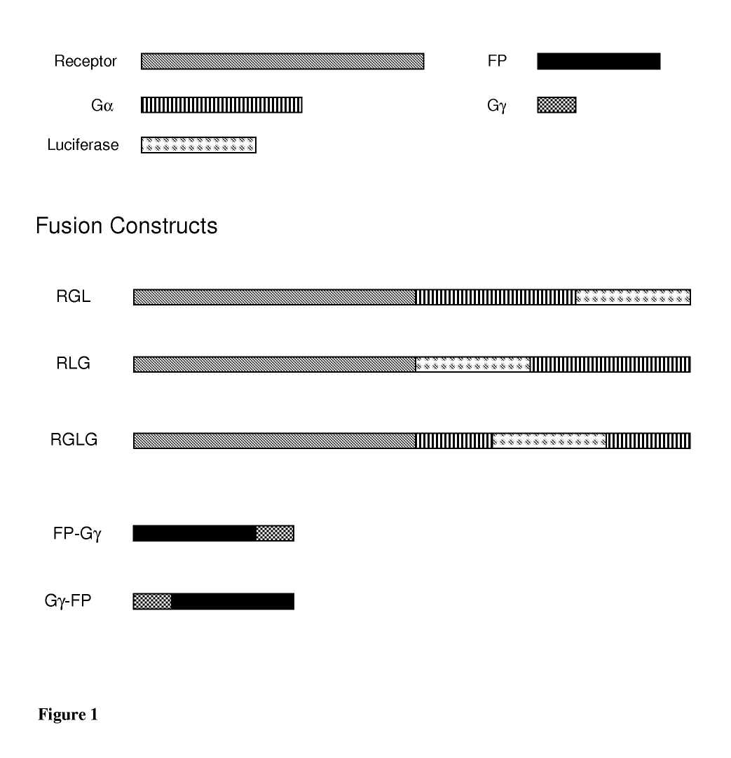

Several BRET systems comprising two separate partners (constructs) were prepared: RLG or RGL or RGLG and, various Gγ-FP, FP-Gγ constructs, in general Gγ9 was used (FIG. 1; Table 5). In these examples, the abbreviations RLG, RGL and FP may be used, in which “R” stands for receptor, “G” for G protein and “L” for luciferase. FP in Gγ9-FP or FP-Gγ9, for example, means fluorescent protein. Each construct contains a protein fused to either the luciferase or the fluorescent protein.

Table 5 and below summarizes the examples of the present specification and also indicates the figures that were obtained with assays using cells expressing the constructs of the invention. Examples 1-12 exemplify the preparations of constructs as detailed below. The Examples following Example 12 are all based on cells co-expressing the constructs as described in Table 5, in which further receptors and/or different construct types (RLG, RGL, RGLG1-3) were used. The constructs were obtained in the same manner as exemplified in Examples 1-12.

TABLE 5
Examples 1-47

| | | Construct | | SEQ ID | Gγ | SEQ ID | |
|---|---|---|---|---|---|---|---|
| Example | GPCR | type | Gα | NO | construct | NO | Figures |
| 1 | | | | | FP-Gγ9 | 3, 4 | |
| 2 | | | | | Gγ9-FP | 5, 6 | |
| 3 | GLP-1R | RLG | Gαs | 7, 8 | | | |
| 4 | hGIPR | RLG | Gαs | 9, 10 | | | |
| 5 | hA2AR | RLG | Gαs | 11, 12 | | | |
| 6 | GLP-1R | RGL | Gαs | 13, 14 | | | |
| 7 | hGIPR | RGL | Gαs | 15, 16 | | | |
| 8 | hA2AR | RGL | Gαs | 17, 18 | | | |
| 9 | GLP-1R | RGLG-1 | Gαs | 19, 20 | | | |
| 10 | hGIPR | RGLG-1 | Gαs | 21, 22 | | | |
| 11 | hA2AR | RGLG-1 | Gαs | 23, 24 | | | |
| 12 | hM4-R | RGLG-2 | Gαo | 83, 84 | | | |
| 13 | | | | | FP-Gγ9 | 3, 4 | |
| 14 | | | | | Gγ9-FP | 5, 6 | |
| 15 | GLP-1R | RLG | Gαs | 7, 8 | FP-Gγ9 | 3, 4 | 5A, 19 |
| 16 | GLP-1R | RLG | Gαs | 7, 8 | Gγ9-FP | 5, 6 | |
| 17 | hGIPR | RLG | Gαs | 9, 10 | FP-Gγ9 | 3, 4 | 5B, 6, 7B, 8 |
| 18 | hGIPR | RLG | Gαs | 9, 10 | Gγ9-FP | 5, 6 | 3B |
| 19 | hA2AR | RLG | Gαs | 11, 12 | FP-Gγ9 | 3, 4 | |
| 20 | hA2AR | RLG | Gαs | 11, 12 | Gγ9-FP | 5, 6 | |
| 21 | GLP-1R | RGL | Gαs | 13, 14 | FP-Gγ9 | 3, 4 | |
| 22 | GLP-1R | RGL | Gαs | 13, 14 | Gγ9-FP | 5, 6 | |
| 23 | hGIPR | RGL | Gαs | 15, 16 | FP-Gγ9 | 3, 4 | |
| 24 | hGIPR | RGL | Gαs | 15, 16 | Gγ9-FP | 5, 6 | 3A |
| 25 | hA2AR | RGL | Gαs | 17, 18 | FP-Gγ9 | 3, 4 | 5C, 7A, 9 |
| 26 | hA2AR | RGL | Gαs | 17, 18 | Gγ9-FP | 5, 6 | |
| 27 | GLP-1R | RGLG-1 | Gαs | 19, 20 | FP-Gγ9 | 3, 4 | |
| 28 | GLP-1R | RGLG-1 | Gαs | 19, 20 | Gγ9-FP | 5, 6 | |
| 29 | hGIPR | RGLG-1 | Gαs | 21, 22 | FP-Gγ9 | 3, 4 | |
| 30 | hGIPR | RGLG-1 | Gαs | 21, 22 | Gγ9-FP | 5, 6 | |
| 31 | hA2AR | RGLG-1 | Gαs | 23, 24 | FP-Gγ9 | 3, 4 | |
| 32 | hA2AR | RGLG-1 | Gαs | 23, 24 | Gγ9-FP | 5, 6 | |
| 33 | hM4-R | RGLG-2 | Gαo | 83, 84 | FP-Gγ9 | 3, 4 | 4, 7C, 10A, |
| | | | | | | | 10B, 10C, 20 |
| 34 | hM4-R | RGLG-2 | Gαo | 83, 84 | Gγ9-FP | 5, 6 | |
| 35 | hM1R | RGLG-3 | Gαq | 86, 87 | FP-Gγ9 | 3, 4 | 11A, 11B |
| 36 | hM2R | RGLG-2 | Gαo | 88, 89 | FP-Gγ9 | 3, 4 | 12 |
| 37 | hGHSR | RLG | Gαq | 90, 91 | FP-Gγ9 | 3, 4 | 13 |
| 38 | hGLP2R | RLG | Gαs | 92, 93 | FP-Gγ9 | 3, 4 | 14 |
| 39 | GCGR | RLG | Gαs | 94, 95 | FP-Gγ9 | 3, 4 | 15 |
| 40 | mGluR2 | RGLG-2 | Gαo | 96, 97 | FP-Gγ9 | 3, 4 | 16 |
| 41 | hOx1R | RLG | Gαq | 98, 99 | FP-Gγ9 | 3, 4 | 17 |
| 42 | hOx2R | RLG | Gαq | 100, 101 | FP-Gγ9 | 3, 4 | 18 |
| 43 | Tables 7, 8 | | | | Tables 7, 8 | | 19, 20 |
| 44 | GLP-1R | RLG | Gαs | 7, 8 | FP-Gγ9 | 3, 4 | 21, 22 |
| 45 | GLP-1R | RLG | Gαs | 7, 8 | FP-Gγ9 | 3, 4 | 23, 24 |
| 46 | hM4-R | RGLG-2 | Gαo | 83, 84 | FP-Gγ9 | 3, 4 | Table 10 |
| 47 | hM4-R | RGLG-2 | Gαo | 83, 84 | FP-Gγ9 | 3, 4 | 25, 26 |

TABLE 6
Different GLG constructs for introducing a Luminescent
protein (L) within the Gα subunit depending
on the type of Gα protein used (FIG. 2 C)

| | Gαs | Gαo | Gαq | Gαi |
|---|---|---|---|---|
| GLG1 | Gαs¹⁻⁷¹-L- | | Gαq¹⁻⁶⁶-L- | Gαq¹⁻⁶⁰-L- |
| | Gαs⁸²⁻³⁹⁴ | | Gαs⁶⁷⁻³⁵⁹ | Gαs⁶¹⁻³⁵⁴ |
| GLG2 | Gαs¹⁻¹¹⁵-L- | Gαo¹⁻⁹¹-L- | Gαq¹⁻⁹⁷-L- | Gαq¹⁻⁹¹-L- |
| | Gαs¹¹⁶⁻³⁹⁴ | Gαo⁹²⁻³⁵⁴ | Gαs⁹⁸⁻³⁵⁹ | Gαs⁹²⁻³⁵⁴ |
| GLG3 | Gαs¹⁻¹⁴⁰-L- | | Gαq¹⁻¹²⁴-L- | Gαq¹⁻¹²²-L- |
| | Gαs¹⁴¹⁻³⁹⁴ | | Gαs¹²⁵⁻³⁵⁹ | Gαs¹²³⁻³⁵⁴ |

### Examples 1 and 2

### Preparation of FPGγ9 and Gγ9FP constructs 1 and 2 (FP-Gγ9 and Gγ9-FP)

Human G-protein gamma9 nucleotide sequence was amplified from pcDNA3.1, Incyte Genomics (clone ID: GNG0900000). The yellow fluorescent protein YPet (A. Nguyen and P. Daugherty, in “Evolutionary optimization of fluorescent proteins for intracellular FRET”, Nature Biotechnology, vol. 23, no. 3, pp. 355-360) was also amplified from pcDNA (SEQ. ID. NO. 2).

The fusion protein Gγ9-YPet and YPet-Gγ9 nucleotide sequences were amplified by sequential PCR reactions, combining Gateway™ Cloning Technology (Invitrogen) and fusion PCR using the primers given below. The fusion primers contain regions that allow the Gγ9 DNA sequences to be fused with the fluorescent protein either on its C- or N-terminus. The Gateway™ Cloning primers contain regions that allow the integration of the fusion proteins into the Gateway™ Cloning vector pDONR™221 (Gateway™ Cloning Technology, Invitrogen).

FP primers for FP-Gγ9 fusion: Primer 1 and primer 2 (SEQ. ID. NOs. 25 and 26, respectively).

Gγ9 primers for FP-Gγ9 fusion: Primer 3 and primer 4 (SEQ. ID. NOs. 27 and 28).

The obtained PCR products from these reactions contain overlapping regions. The PCR products were purified and fused by re-amplifying a mix of the two products with primers 1

(SEQ. ID. NO. 25) and 4 (SEQ. ID. NO. 28). The resulting fusion protein FP-Gγ9 was then put into pDONR™221 (Gateway™ Cloning Technology, Invitrogen) according to manufacturer's instructions.

FP primers for Gγ9-FP construct: Primer 5 and primer 6 (SEQ. ID. NOs. 29 and 30).

Gγ9 primers for Gγ9-FP construct: Primer 7 and primer 8 (SEQ. ID. NOs. 31 and 32)

In analogy to the FP-Gγ9 construct above, the obtained PCR products were purified and fused by re-amplifying a mix of the two products with primers 6 and 7 (SEQ. ID. NOs. 30 and 31). The resulting fusion protein Gγ9-FP was then put into pDONR™221, according to manufacturer's instructions.

Finally, both fusion proteins were subcloned into the doxycycline-inducible target expression plasmid pTRE2-hygromycin vector containing Gateway recombination sites (pTRE2-hygromycin-FP-Gγ9, and pTRE2-hygromycin-Gγ9-FP).

SEQ. ID. NOs.: 3-6 are nucleotide (NOs. 3, 5) and amino acid (NOs. 4, 6) sequences of the FP-Gγ9 (NOs. 3, 4) and Gγ9-FP(NOs. 5, 6) fusion products (constructs 1 and 2).

### Example 3

### Preparation of the GPCR-Luciferase-Gαs Construct 1 (RLG)

The GPCR-Luciferase-Gαs construct prepared in this example contains three different domains: human GLP-1 receptor (hGLP-1R), a mutated Renilla luciferase (SEQ. ID. NO.:1), and the human G-protein subunit Gαs long (accession number NM_{—}000516), all of which were fused in this order in the amino to the carboxy direction.

hGLP-1R DNA was PCR amplified from the human hypothalamus cDNA (Clontech, cat. nr. 639329) using primers 9 and 10 (SEQ. ID. NOs. 33 and 34). The obtained DNA was afterwards sequenced, put in the pTRE2-hygromycin vector and sequenced again.

Thereafter, the human GLP-1 receptor sequence without the stop codon was put into a Multisite Gateway pDONR™221P1-P4 vector (MultiSite Gateway® Pro Cloning Technology, Invitrogen), in accordance with the manufacturer's instructions using the following primers: Primer 11 and primer 12 (SEQ. ID. NOs. 35 and 36).

The mutated Renilla luciferase, the second element of the fusion protein, was amplified without stop codon from SEQ. ID. NO.: 1 (Reference: Lorenz et al, 1991, PNAS 88, pp 4438-4442. “Isolation and expression of a cDNA encoding Renilla reniformis luciferase”) and was put into a Multisite Gateway pDONR™221 P4r-P3r vector (MultiSite Gateway® Pro Cloning Technology, Invitrogen) in accordance with the manufacturer's instructions using primers 13 and 14 (SEQ. ID. NOs. 37 and 38).

G-protein subunit Gαs (long isoform, accession number NM_{—}000516), the third element of the fusion protein, was amplified from SH-SY5Y cells cDNA that was obtained by reverse transcription from SH-SY5Y cells (ATCC Number CRL-2266) using primers 15 and 16 (SEQ. ID. NOs. 39 and 40).

Thereafter, the amplified sequence was put into a pCR®II-TOPO® vector (TOPO® TA Cloning® Kit, Invitrogen), in accordance with the manufacturer's instructions. This sequence was put into a Multisite Gateway pDONR™221P3-P2 vector (MultiSite Gateway® Pro Cloning Technology, Invitrogen), in accordance with the manufacturer's instructions using primers 17 and 18 (SEQ. ID. NOs. 41 and 42).

The three elements, hGLP-1R, Renilla luciferase, and Gαs long in their respective MultiSite Gateway® Pro pDONR™211 vectors were fused and inserted into a pTRE2 target vector containing the appropriate recombination sites by performing a recombination reaction in accordance with the manufacturer's instructions, as illustrated in FIG. 2 A. The resulting doxycycline-inducible pTRE2-puromycin expression plasmid (pTRE2-puromycin-hGLP-1R-L-G) contains the fused human GLP-1R-Renilla luciferase-Gαs long construct.

SEQ. ID. NOs: 7 and 8 are the nucleotide and amino acid sequences, respectively, of the resulting construct and fusion protein of the type RLG (FIG. 1).

Through the recombination process, linker motifs of 9 (hptflykvg) and 8 (ttlynkva) amino acids (SEQ. ID. NO. 72 and 73) that separate the protein domains were inserted into the fusion protein.

### Example 4

### Preparation of GPCR-Luciferase-Gαs Construct 2 (RLG)

The GPCR-Luciferase-Gαs construct prepared in this example differs from the one in Example 3 in that the GPCR is human GIP receptor (hGIPR, accession number NM_{—}000164) instead of hGLP-1R. The remaining components of the fusion protein are the same as in Example 3.

Human GIP receptor DNA was amplified from the human hippocampus cDNA (Clontech, cat. nr. 637228) using PCR (classical molecular biology techniques) with primers 19 and 20 (SEQ. ID. NOs. 43 and 44).

The obtained DNA was afterwards sequenced, put in the pTRE2-hygromycin vector and sequenced again.

Thereafter, the human GIP receptor sequence without the stop codon was put into a Multisite Gateway pDONR™221P1-P4 vector, in accordance with the manufacturer's instructions using primers 21 and 22 (SEQ. ID. NOs. 45 and 46).

The three elements, the hGIPR, the human G-protein subunit Gαs long (pDONR™221 P3-P2, Example 3) and Renilla luciferase in their respective pDONR™221 vectors (pDONR™221P1-P4 above and pDONR™221 P4r-P3r and pDONR™221P3-P2 of Example 3) were used for the recombination reaction in exactly the same way as described in Example 3 and shown in FIG. 2 A. The resulting doxycycline-inducible pTRE2-puromycin expression plasmid (pTRE2-puromycin-hGIPR-L-G) contains the fused human GIPR-Renilla luciferase—Gαs long construct.

SEQ. ID. NOs. 9 and 10 are the nucleotide and amino acid sequences, respectively, of the obtained construct and fusion protein of the type RLG (FIG. 1).

The same linker motifs separating the protein domains as described in Example 3 were inserted into the fusion protein.

### Example 5

### Preparation of the GPCR-Luciferase-Gαs Construct 3 (RLG)

The GPCR-Luciferase-Gαs construct prepared in this example differs from the one in Examples 3 and 4 in that the GPCR is human A_{2A} receptor (hA_{2A}R, accession number NP_{—}000666) instead of hGLP-1R and hGIPR, respectively. The remaining components of the fusion protein are the same as in Examples 3 and 4.

Human A_{2A} receptor DNA was amplified from the human heart cDNA (Clontech, cat. nr. 637213) using PCR (classical molecular biology techniques) with primers 23 and 24 (SEQ. ID. NOs. 47 and 48).

The obtained DNA was afterwards sequenced, put in the pTRE2-hygromycin vector and sequenced again.

Thereafter, the human A_{2A} receptor sequence without the stop codon was put into a Multisite Gateway pDONR™221P1-P4 vector, in accordance with the manufacturer's instructions using primers 25 and 26 (SEQ. ID. NOs. 49 and 50).

The three elements, the hA_{2A}R, the human G-protein subunit Gαs long (pDONR™221 P3-P2, Example 3) and Renilla luciferase in their respective pDONR™221 vectors (pDONR™221P1-P4 above and pDONR™221 P4r-P3r and pDONR™221P3-P2 of Example 3) were used for the recombination reaction in exactly the same way as described in Example 3 and shown in FIG. 2 A. The resulting doxycycline-inducible pTRE2-puromycin expression plasmid (pTRE2-puromycin-hA_{2A}R-L-G) contains the fused human A_{2A}R-Renilla luciferase-Gαs long construct.

SEQ. ID. NOs. 11 and 12 are the nucleotide and amino acid sequences, respectively, of the obtained construct and fusion protein of the type RLG (FIG. 1).

The same linker motifs separating the protein domains as described in Example 3 were inserted into the fusion protein.

### Example 6

### Preparation of GPCR-Gαs-Luciferase Construct 1 (RGL)

The GPCR-Gαs-Luciferase construct prepared in this example contains three different domains: human GLP-1 receptor, human G-protein subunit Gαs long and Renilla luciferase, all of which were fused, in this order in the amino to the carboxy direction.

The pDONR™221P1-P4 vector containing hGLP-1R prepared in Example 3 was also used in this example for the final recombination reaction.

The amplified sequence of human G-protein subunit Gαs long (Example 3) without the stop codon was put into a Gateway pDONR™221P4r-P3r vector, in accordance with the manufacturer's instructions using primers 27 and 28 (SEQ. ID. NOs. 51 and 52).

The amplified sequence of Renilla luciferase (Example 3) was put into a Gateway pDONR™221 P3-P2 vector, in accordance with the manufacturer's instructions using the Gateway primers 29 and 30 (SEQ. ID. NOs. 53 and 54).

The three elements, hGLP-1R, Renilla luciferase, and Gαs long in their respective Gateway pDONR™211 vectors were fused and inserted into a pTRE2 target vector containing the appropriate recombination sites by performing a recombination reaction in accordance with the manufacturer's instructions, as illustrated in FIG. 2 B. The resulting doxycycline-inducible pTRE2-puromycin expression plasmid (pTRE2-puro-hGLP-1R-G-L) contains the fused hGLP-1R-Gαs-Luciferase construct (RGL-1).

SEQ. ID. NOs. 13 and 14 are the nucleotide and amino acid sequences, respectively, of the obtained construct and fusion protein of the type RGL (FIG. 2 B). The same linker motifs separating the protein domains as described in Example 3 were inserted into the fusion protein.

### Example 7

### Preparation of GPCR-Gαs-Luciferase Construct 2 (RGL)

The GPCR-Gαs-Luciferase construct prepared in this example differs from the one in Example 6 in that the GPCR is human GIP receptor, instead of hGLP-1R. The remaining components of the fusion protein are the same as in Example 6.

The sources of human G-protein subunit Gαs long and Renilla luciferase are indicated in Example 3 and of the human GIP receptor in Example 4.

For the recombination reaction, the Gateway pDONR™221 P1-P4 vector containing the hGIPR sequence prepared in Example 4, and the Gateway pDONR™221P4r-P3r and pDONR™221 P3-P2 vectors containing Gαs long and Renilla luciferase, respectively, prepared in Example 6, were used.

The three elements, hGIPR, Gαs and Renilla luciferase, in their respective Gateway pDONR™221 vectors were fused and inserted into a pTRE2 target vector as described above (FIG. 2 B). The resulting doxycycline-inducible pTRE2-puromycin expression plasmid (pTRE2-puromycin-hGIPR-G-L) contains the fused hGIPR-Gαs-Luciferase construct (RGL-2).

SEQ. ID. NOs. 15 and 16 are the nucleotide and amino acid sequences, respectively, of the construct and fusion protein, which is of the type RGL (FIG. 1). The same linker motifs separating the protein domains as described in Example 3 were inserted into the fusion protein.

### Example 8

### Preparation of GPCR-Gαs-Luciferase Construct 3 (RGL-3)

The GPCR-Gαs-Luciferase construct prepared in this example differs from ones in Examples 6 and 7 in that the GPCR is human A_{2A} receptor, instead of hGLP-1R and hGIPR, respectively. The remaining components of the fusion protein are the same as in Example 6.

The sources of human G-protein subunit Gαs long and Renilla luciferase are indicated in Example 3 and of the human A_{2A} receptor in Example 5.

For the recombination reaction, the Gateway pDONR™221 P1-P4 vector containing the hA_{2A}R sequence prepared in Example 5, and the Gateway pDONR™221 P4r-P3r and pDONR™221 P3-P2 vectors containing Gαs long and Renilla luciferase, respectively, prepared in Example 6, were used.

The three elements, hA_{2A}R, Gαs and Renilla luciferase, in their respective Gateway pDONR™221 vectors were fused and inserted into a pTRE2 target vector as described above (FIG. 2 B). The resulting doxycycline-inducible pTRE2-puromycin expression plasmid (pTRE2-puromycin-hA_{2A}R-G-L) contains the fused hA_{2A}R-Gαs-Luciferase construct (RGL-3).

SEQ. ID. NOs. 17 and 18 are the nucleotide and amino acid sequences, respectively, of the construct and fusion protein (type RGL, FIG. 1). The same linker motifs separating the protein domains as described in Example 3 were inserted into the fusion protein.

### Example 9

### Preparation of GPCR-Gαs^{t-71}-Luciferase-Gαs⁸²⁻³⁹⁴ Construct 1 (RGLG-1)

The GPCR-Gαs^{t-71}-Luciferase-Gαs⁸²⁻³⁹⁴ construct prepared in this example contains four different domains: human GLP-1 receptor, amino acids 1-71 of human G-protein subunit Gαs long (Gαs^{s-71}), mutated Renilla luciferase and amino acids 82-394 of human G-protein subunit Gαs long (Gαs⁸²⁻³⁹⁴), all of which were fused in this order in the amino to the carboxy direction.

The first step was the making of the Gαs^{t-71}-Luciferase-Gαs⁸²⁻³⁹⁴ (GLG1) fusion construct using the MultiSite Gateway Pro® system, the construct being fused afterwards to the hGLP-1R using classical cloning techniques (two-step fusion PCR).

The same human G-protein subunit Gαs long and mutated Renilla luciferase as in Example 3 were used.

The nucleotide sequence Gαs¹⁻⁷¹ was amplified and put in the pDONR™221 P1-P4 vector using primers 31 and 32 (SEQ. ID. NOs. 55 and 56).

The nucleotide sequence of mutated Renilla luciferase was amplified and put in the pDONR™221 P4r-P3r using primers 33 and 34 (SEQ. ID. NOs. 57 and 58).

The sequence Gαs⁸²⁻³⁹⁴ was amplified and put in the pDONR™221 P3-P2 vector using primers 35 and 36 (SEQ. ID. NOs. 59 and 60).

The three pDONR™221 vectors were then recombined by a MultiSite Gateway recombination reaction to obtain a doxycycline-inducible pTRE2-puromycin vector (pTRE2-puromycin-Gas¹⁻⁷¹-L-Gas⁸²⁻³⁹⁴) for the expression of Gαs^{t-71}-Luciferase-Gαs⁸²⁻³⁹⁴ (GLG1), as illustrated in FIG. 2 C. Through the recombination process, the linker motifs of SEQ.

ID. NOs. 72 and 73 from Example 3 separating the protein domains were inserted into the fusion protein (between Gαs^{t-71}-Luciferase and Luciferase-Gαs⁸²⁻³⁹⁴, respectively).

The fusion of the Gαs¹⁻⁷¹-Luciferase-Gαs⁸²⁻³⁹⁴ sequence with the hGLP-1R was done by classical two-step fusion PCR using the same principle as described in Examples 1 and 2.

Accordingly, the hGLP-1R DNA (Example 3) was amplified using primers 37 and 38 (SEQ. ID. NOs. 61 and 62).

The Gαs^{t-71}-Luciferase-Gαs⁸²⁻³⁹⁴ (GLG-1) construct was amplified from the pTRE2-puromycin-Gαs¹⁻⁷¹-L-GUS⁸²⁻³⁹⁴ vector above using primers 39 and 36 (SEQ. ID. NOs. 63 and 60, respectively).

In the second PCR reaction, the two PCR products were fused using primers 37 and 40. The purified PCR product (hGLP-1R-Gαs¹⁻⁷¹-Luciferase-Gαs⁸²⁻³⁹⁴) was cloned into pDONR™221, in accordance with the manufacturer's instructions.

This vector was afterwards recombined with the mammalian doxycycline-inducible pTRE2-puromycin expression vector to give pTRE2-puromycin-hGLP-1R-G¹⁻⁷¹-L-G⁸²⁻³⁹⁴ containing the fused hGLP-1R-Gαs¹⁻⁷¹-Luciferase-GUS⁸²⁻³⁹⁴ construct (RGLG-1).

SEQ. ID. NOs. 19 and 20 are the nucleotide and amino acid sequences, respectively, of the construct and fusion protein (type RGLG-1, FIG. 2C).

### Example 10

### Preparation of GPCR-Gαs^{t-71}-Luciferase-Gαs⁸²⁻³⁹⁴ Construct 2 (RGLG-1)

The GPCR-Gαs^{t-71}-Luciferase-Gαs⁸²⁻³⁹⁴ construct prepared in this example differs from the one in Example 7 in that the GPCR is human GIP receptor instead of hGLP-1R. The remaining components of the fusion protein are the same as in Example 7.

The fusion protein DNA Gαs¹⁻⁷¹-Luciferase-Gαs⁸²⁻³⁹⁴ prepared in Example 7 and the human GIP receptor DNA disclosed in Example 4 were used. The fusion of the hGIP-R DNA with Gαs^{t-71}-Luciferase-Gαs⁸²⁻³⁹⁴ DNA was done by a two-step fusion PCR reaction (Examples 1, and 9).

The hGIP-R sequence was amplified using primer 21 (Example 4, SEQ. ID. NO. 45) and primer 40 (SEQ. ID. NO. 64).

The Gαs^{t-71}-Luciferase-Gαs⁸²⁻³⁹⁴ sequence (Example 7) was amplified using primer 41 and primer 40 (SEQ. ID. NOs. 65 and 64, respectively).

In the second PCR reaction, the two PCR products were fused using primers 21 (SEQ. ID. NO. 45 from Example 4) and 36 (SEQ. ID. NO. 60 from Example 9). The purified PCR product (hGIPR—Gαs¹⁻⁷¹—Luciferase-Gαs⁸²⁻³⁹⁴) was cloned into pDONR™221, in accordance with the manufacturer's instructions. This vector was afterwards recombined with the mammalian doxycycline-inducible pTRE2-puromycin expression vector to yield pTRE2-puromycin-hGIP-G¹⁻⁷¹-L-G⁸²⁻³⁹⁴ containing the fused hGIPR-Gαs¹⁻⁷¹-Luciferase-GUS⁸²⁻³⁹⁴ construct (RGLG-2).

SEQ. ID. NOs. 21 and 22 are the nucleotide and amino acid sequences, respectively, of the construct and fusion protein (type RGLG-1, FIGS. 1, 2C).

### Example 11

### Preparation of GPCR-Gαs^{t-71}-Luciferase-Gαs⁸²⁻³⁹⁴ Construct 3 (RGLG-1)

The GPCR-Gαs^{t-71}-Luciferase-Gαs⁸²⁻³⁹⁴ construct prepared in this example differs from the one in Example 9 and 10 in that the GPCR is human A_{2A} receptor (hA_{2A}R, accession number NP_{—}000666) instead of hGLP-1R or hGIPR, respectively. The remaining components of the fusion protein are the same as in Example 9.

The fusion protein DNA Gαs¹⁻⁷¹-Luciferase-Gαs⁸²⁻³⁹⁴ prepared in Example 9 and the hA_{2A}R DNA disclosed in Example 5 were used. The fusion of the hA_{2A}R DNA with Gαs¹⁻⁷¹-Luciferase-Gαs⁸²⁻³⁹⁴ DNA was done by a two-step fusion PCR reaction (Examples 1, 2, 9 and 10).

The hA_{2A}R sequence with the overlapping sequence of Gαs was amplified using primers 42 and 43 (SEQ. ID. NOs. 66 and 67).

The Gαs^{t-71}-Luciferase-Gαs⁸²⁻³⁹⁴ sequence (Example 9) was amplified using primers 44 and 45 (SEQ. ID. NOs. 68 and 69).

In the second PCR reaction, the two PCR products were fused using primers 46 and 47 (SEQ. ID: NOs.: 70 and 71).

The purified PCR product (hA_{2A}R-Gαs¹⁻⁷¹-Luciferase-Gαs⁸²⁻³⁹⁴) was cloned into pDONR™221, in accordance with the manufacturer's instructions. This vector was afterwards recombined with the mammalian doxycycline-inducible pTRE2-puromycin expression vector to yield pTRE2-puromycin-hA_{2A}R-G¹⁻⁷¹-L-G⁸²⁻³⁹⁴ containing the fused hGIPR-Gαs¹⁻⁷¹-Luciferase-GUS⁸²⁻³⁹⁴ construct (RGLG-3).

SEQ. ID. NOs. 23 and 24 are the nucleotide and amino acid sequences, respectively, of the obtained construct and fusion protein (type RGLG-1, FIGS. 1, 2C).

### Example 12

### Preparation of GPCR-Gαo¹⁻⁹¹-Luciferase-Gαo⁹²⁻³⁵⁴ Construct (RGLG-2)

The GPCR-Gαo¹⁻⁹¹-Luciferase-Gaso⁹²⁻³⁵⁴ construct prepared in this example contains four different domains: human muscarinic acetylcholine receptor M₄, amino acids 1-91 of human G-protein Gαo (Gαo¹⁻⁹¹), mutated Renilla luciferase and amino acids 92-354 of human G-protein Gαo (Gαo⁹²⁻³⁵⁴), all of which were fused in this order in the amino to the carboxy direction. During PCR flexible SGGGGS-linkers (SEQ. ID. NO.: 85) were inserted between the M₄-receptor and Gαo as well as between Gαo and Rluc8 by the use of primers carrying the linker sequences (primers 50-52, 55 and 56).

The first step was the making of the Gαo¹⁻⁹¹-Luciferase-Gαo⁹²⁻³⁵⁴ fusion construct using the MultiSite Gateway Pro® system, the construct being fused afterwards to the hM₄-R using classical cloning techniques (two-step fusion PCR).

The same mutated Renilla luciferase as in Example 3 was used. Human M₄-Receptor cDNA was purchased from Open Biosystems (cat. nr. MHS1768-99865636). The human Goo protein (accession number NM_{—}020988) was amplified from human whole brain cDNA (Clontech, cat. nr. 637242) using primers 48 and 49 (SEQ. ID. NOs: 74 and 75).

The nucleotide sequence of Gαo¹⁻⁹¹ was amplified and put in the pDONR™221 P1-P4 vector using primers 50 and 51 (SEQ. ID. NOs. 76 and 77).

The nucleotide sequence of mutated Renilla luciferase was amplified and put in the pDONR™221 P4r-P3r using primers 33 and 34 (The same vector as used in Example 9).

The sequence Gαo⁹²⁻³⁵⁴ was amplified and put in the pDONR™221 P3-P2 vector using primers 52 and 53 (SEQ. ID. NOs.: 78 and 79).

The three pDONR™221 vectors were then recombined by a MultiSite Gateway recombination reaction to obtain a doxycycline-inducible pTRE2-puromycin vector (pTRE2-puromycin-Gαo¹⁻⁹¹-L-Gαo⁹²⁻³⁵⁴) for the expression of Gαo¹⁻⁹¹-Luciferase-Gαo⁹²⁻³⁵⁴ (GLG2), as illustrated in FIG. 2D. Through the recombination process, the linker motifs of 9 and 8 amino acids (SEQ. ID. NO. 72 and 73 from Example 3) separating the protein domains were inserted into the fusion protein (between Gαo¹⁻⁹¹-Luciferase and Luciferase-Gαo⁹²⁻³⁵⁴, respectively).

The fusion of the Gαo¹⁻⁹¹-Luciferase-Gαo⁹²⁻³⁵⁴ sequence with the hM₄-R was done by classical two-step fusion PCR using the same principle as described in Examples 1 and 2.

Accordingly, the hM₄-R DNA was amplified using the following primers 54 and 55 (SEQ. ID. NO.: 80 and 81).

The Gαo¹⁻⁹¹-Luciferase-Gαo⁹²⁻³⁵⁴ construct was amplified from the pTRE2-puromycin-Gαo^{1 91}-L-Gαo⁹²⁻³⁵⁴ vector above using primers 56 and 53 (SEQ. ID. NOs.: 82 and 79).

In the second PCR reaction, the two PCR products were fused using primers 54 and 53. The purified PCR product (hM₄-R-Gαo¹⁻⁹¹-Luciferase-Gαo⁹²⁻³⁵⁴) was cloned into pDONR™221, in accordance with the manufacturer's instructions.

This vector was afterwards recombined with the mammalian doxycycline-inducible pTRE2-puromycin expression vector to give pTRE2-puromycin-hM₄-R-G¹⁻⁹¹-L-G⁹²⁻³⁵⁴ containing the fused hM₄-R-Gαo¹⁻⁹¹-Luciferase-GU0⁹²⁻³⁵⁴ construct.

SEQ. ID. NOs. 83 and 84 are the nucleotide and amino acid sequences, respectively, of the obtained construct and fusion protein (type RGLG-2, FIGS. 1, 2C).

Preparation of Cells Suitable for HTS in Accordance with the Invention

### Examples 13-14

### Transfection of HEK Tet-On Cells with FP-Gγ9 and Gγ9-FP Vectors

The vectors pTRE2-hygromycin-FP-Gγ9 (Example 1) and pTRE2-hygromycin-Gγ9-FP (Example 2) were separately stably transfected using PolyFect® Transfection Reagent (Qiagen), according to the manufacturer's protocol, into HEK Tet-On Cell Line cells (Clontech Laboratories, Inc.). This cell line is a neomycin resistant human transformed primary embryonal kidney (HEK)-derived cell line transformed with a modified version of pTet-On, that has a nuclear localization signal fused to the N-terminus (pUHD17-lneo).

### Examples 15-34

### Further Transfections with the Vectors Obtained in Examples 3-12

Subsequently, positively transfected HEK Tet-On FP-Gγ9 or Gγ9-FP cells were selected by using 400 μg/ml hygromycin in the growth medium during approximately 4 weeks, single clones were re-picked and grown. One stable HEK Tet-On FP-Gγ9 clone and one stable HEK Tet-On Gγ9-FP clone were chosen and again transfected separately using PolyFect® Transfection Reagent (Qiagen), according to the manufacturer's protocol, with any one of the expression vectors as prepared in Examples 3-12. Separate transfected cells were prepared with all vectors/constructs.

The constructs and the corresponding expression vectors are the expression vectors from Examples 3-5, respectively (pTRE2-puromycin-R-L-G, with R (receptor) standing for hGLP-1R, hGIPR, and hA_{2A}R, respectively; the vectors from Examples 6-8 (pTRE2-puromycin-R-G-L); the vectors from Examples 9-11 (pTRE2-puromycin-R-Gαs¹⁻⁷¹-L-Gαs⁸²⁻³⁹⁴) and the vector from Example 4 (pTRE2-puromycin-R-Gαo¹⁻⁹¹-L-Gαo⁹²⁻³⁵⁴).

Positively transfected HEK Tet-On cells were selected by using 10 μg/ml puromycin in the growth medium during approximately 4 weeks, single clones were re-picked, grown and tested for BRET responses as described below.

In total, 20 different clones were obtained.

### Example 35

### General Assay Protocol for Screening

As HTS device, the FLIPR^{TETRA}® system from Molecular Devices, Sunnyvale, U.S.A., was used (www.moleculardevices.com).

The BRET assay of the invention uses cells that stably co-express constructs containing the Renilla Luciferase (one of DNA sequences from examples 3 to 11) and one of the two sequences containing the fluorescent protein (one of DNA sequences from examples 1 and 2). Cells are grown to 80-90% confluency in the incubator at 37° C. and 5% CO₂ in 15 cm dishes in growth medium containing DMEM (Invitrogen #61965026), 10% FCS (tetracyclin negative: Chemie Brunschwig #BRA11-050), Penicillin/Streptomycin (Invitrogen #15040122; 100 U/ml-100 μg/ml), geneticin (Invitrogen #10131019; 100 μg/ml), hygromycin (Invitrogen #10687010; 40 μg/ml), and puromycin (BD #8052-1; 1 μg/ml).

One day prior to the experiment a cell plate (or read plate) is prepared: cells are detached from the 15 cm dish using short incubation with trypsin/EDTA (Invitrogen #25300054) and seeded in black 384-well plates, at 20-30′000 cells seeded per well (50 μl of cell suspension in growth medium/well) induced overnight with doxycycline at 1 μg/ml.

On the day of the experiment, the growth medium is aspirated and replaced by HBSS++(Gibco, #14065) and cells are starved between 30 and 90 minutes at 37° C. Subsequently, they are washed with PBS (Gibco, #70011) and a 600 μM stock solution of coelenterazine H (Dalton Pharma Services, #0005909-86-9A) in 100% ethanol is diluted in HBSS++ and added to each well such that the final concentration of coelenterazine H in each well after the addition of all the reagents is between 3 μM and 12 μM, depending on the cellular system. 5 or 10 minutes after the addition of coelenterazine H to the cells, the read plate is placed in the FLIPR tetra and the measurement begins.

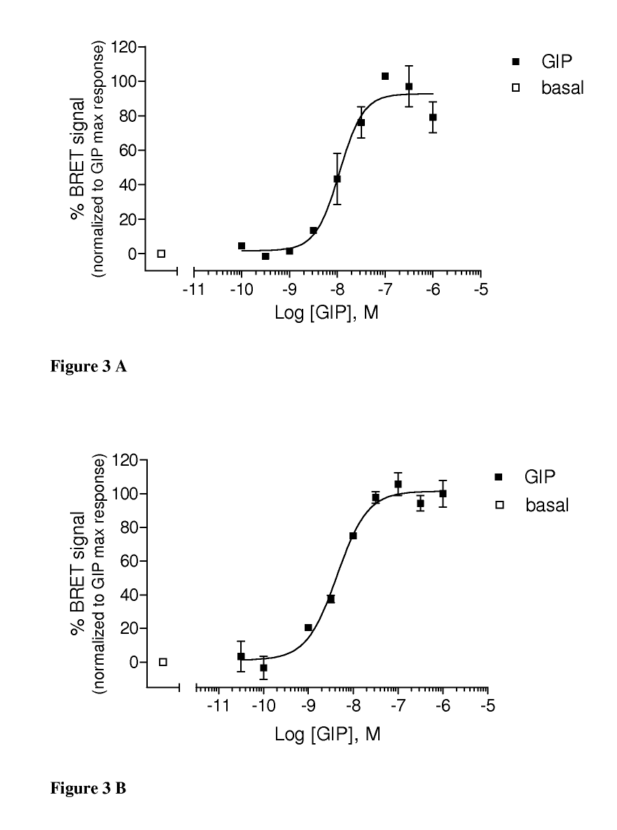

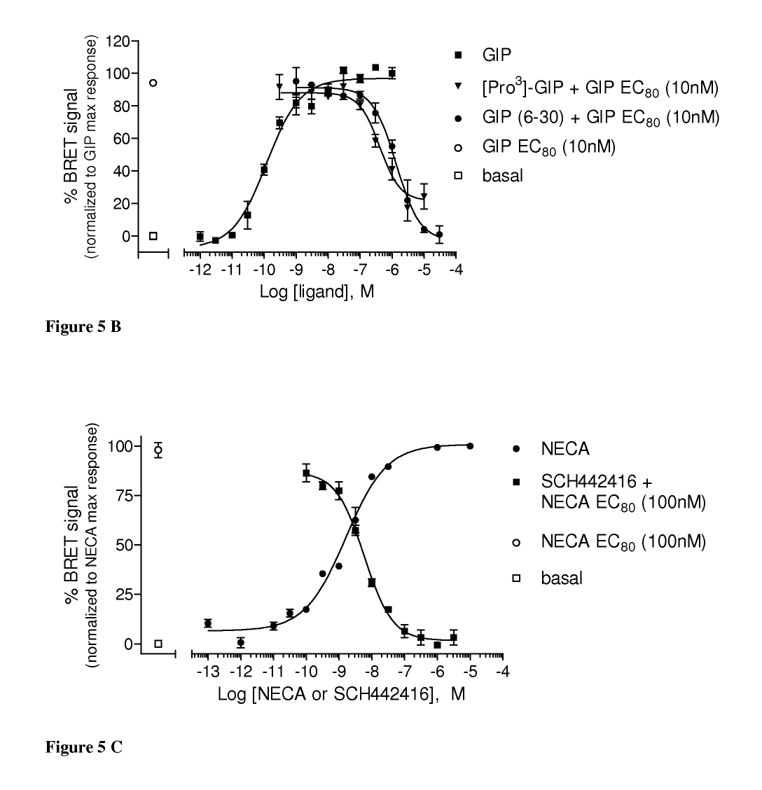

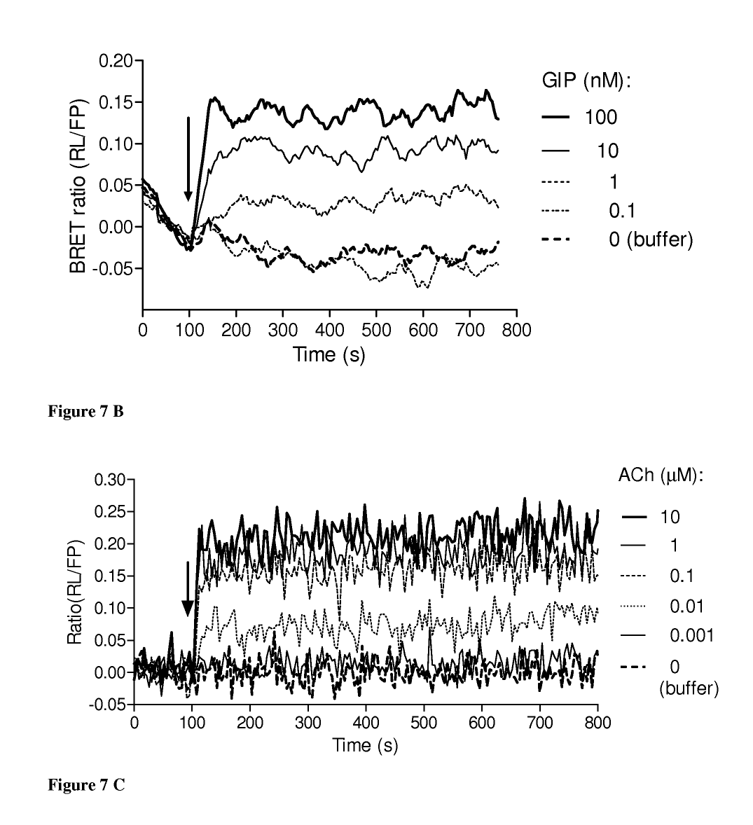

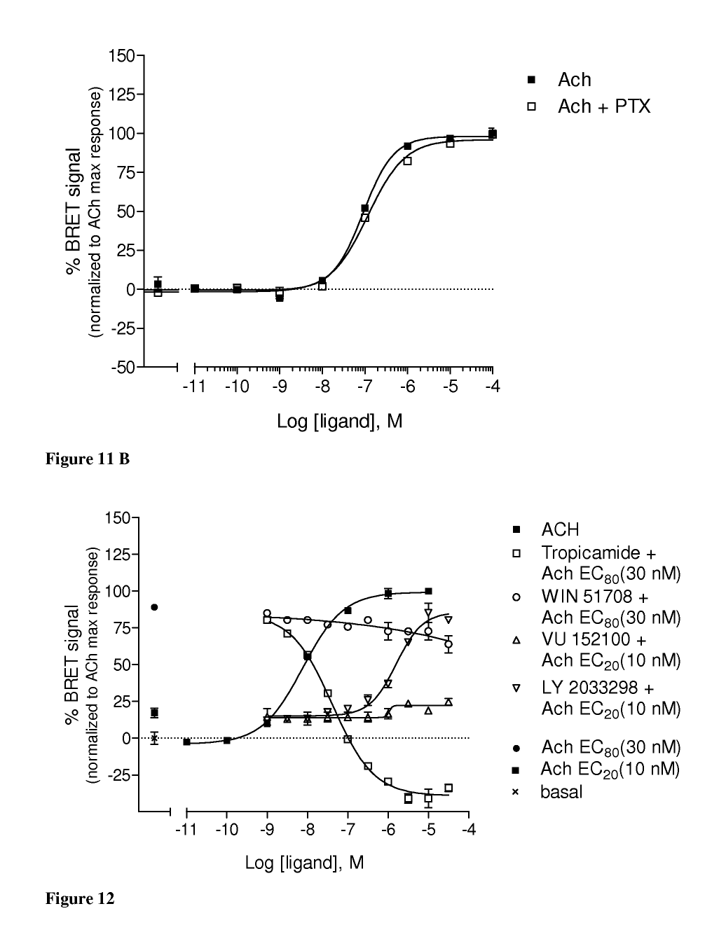

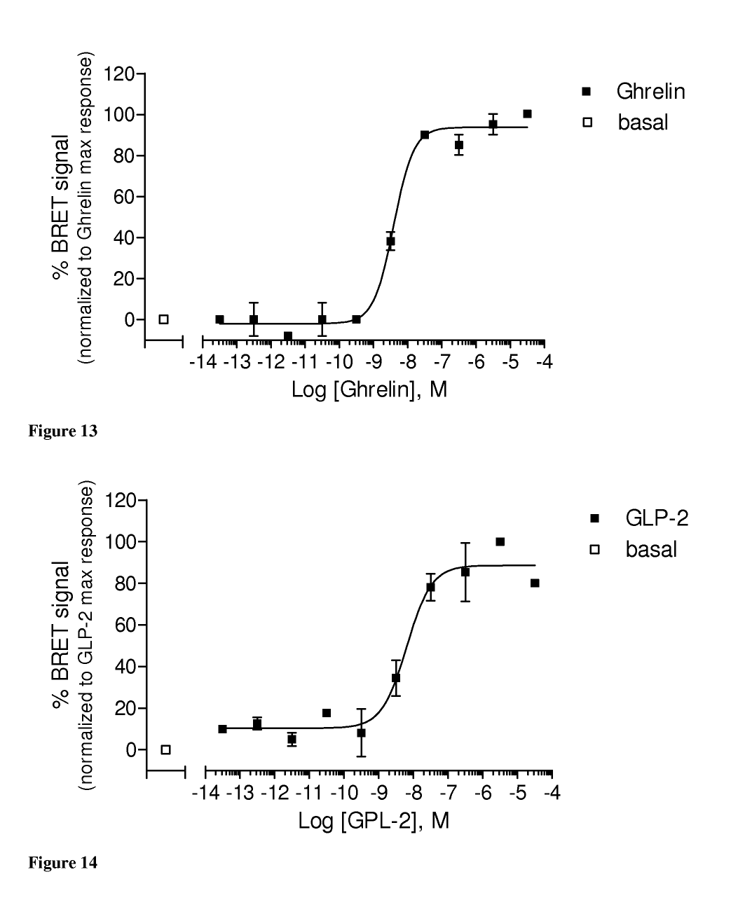

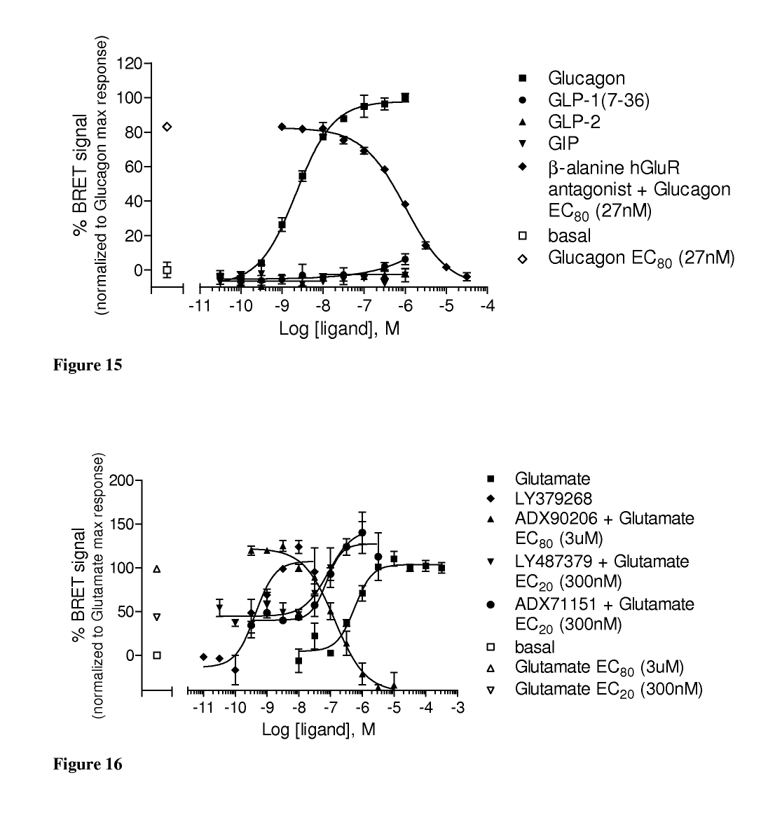

A second plate is a compound plate, containing a mix of an agonist at its EC₂₀ or EC₈₀ and candidate agents to be tested if the protocol run on the FLIPR tetra is a one-addition (or co-addition) protocol (as in FIGS. 3A, 3B, 4, 5A, 6, 7B, 7C, 10B, 10C, 11B, 13, 14, 19 and 20). Alternatively, a two-addition protocol can be run on the FLIPR tetra, containing two compound plates, of which the first one usually contains different candidate agents and the second one an agonist at its EC₂₀ or EC₈₀ (as in FIGS. 5B, 5C, 7A, 9, 10A, 11A, 12, 15, 16, and 18). Finally, a three-addition protocol can also be run on the FLIPR tetra, containing three compound plates, of which the first one usually contains different candidate agents, the second one an agonist at its EC₂₀ and the third one an agonist at its EC₈₀, so that a screen for both positive and negative allosteric modulators can be run at the same time.

The assay is run with the read plate containing the cells located in the read position of the FLIPR Tetra, on top of the detection camera. In positions left and right to the read position, there are places for compound plates. The pipette tip box has a far left position in the FLIPR Tetra. In case of a three addition protocol, the third compound plate is situated in a far left position, while the pipette tips are added manually offline.

Between the transparent bottom of the 384 well read plate and the camera, two emission filters with appropriate bandpass wavelengths to allow light transmission corresponding to spectra of luminescent and fluorescent proteins are installed that move horizontally to let pass sequentially the portion of the light that corresponds to the luminescence emission from the luciferase or the fluorescence emitted by the yellow fluorescent protein.

Immediately before starting the experiment, the amount of light that is emitted from the plate and captured by the camera is determined in a protocol test signal mode that allows the adjustment of the camera exposure time.

The camera is adjusted in such a way that the gain is set to a maximum of 28,000× and the shutters are opened 100%. The signal captured is regulated by adjusting the exposure time to 0.1 to 3 seconds ideally resulting of a signal between 30,000-60,000 RLU. The read interval (emission filter 1, emission filter 2) is adjusted to the minimal value that results from the addition of the two exposure times (=2×0.1 to 3 seconds) plus the time needed to move the filters from position 1 to position 2 (0.3 sec), resulting in a maximal read frequency of 0.5 seconds (2 Hz). A typical experiment has the following sequence.

1) Mixing of the (first) compound plate and loading of the compound into the tips;
2) Reading of light emissions before compound addition: typically between 10 and 30 read intervals;
3) Dispense of the first compound into wells of the read plate;
5) Reading of light emissions;
6) In case of a two-addition protocol: mixing of the second compound plate and loading of the compound into the tips;
7) In case of a two-addition protocol: addition of the compounds on the second compound plate into wells of the read plate;
8) Reading of light emissions;
9) In case of a two-addition protocol: mixing of the third compound plate and loading of the compound into the tips;
10) In case of a two-addition protocol: addition of the compounds on the third compound plate into wells of the read plate;
11) Reading of light emissions;
12) End of protocol.

### Methodology, Results and Z′-Factor of the Screening Experiments

Data from FIGS. 3 A and B were obtained following the one-addition protocol described in Example 35. Specifically, on the day of the experiment, after the 30 minutes starvation in 45 μL HBSS++, 15 μL of 12 μM coelenterazine H was added to each well (final coelenterazine H concentration: 3 μM) and the cell-plate was inserted into the FLIPR tetra reader. Exposure time in both blue and yellow channel was set to 1 second each and a one-addition protocol with a read interval of 3 seconds (one read interval includes both the blue and the yellow channel measurement) was performed. Namely, 20 μL of 4 times concentrated GIP at increasing concentrations, diluted in the HBS++ buffer containing 0.2% BSA, was added to each well after the first six counts and their effect was measured for another 200 counts. Area under the curve of blue/yellow ratio from count 10 to the end of the measurement was taken for the analysis and plotted against GIP concentration for the construction of the graphs in FIGS. 3 A and B.

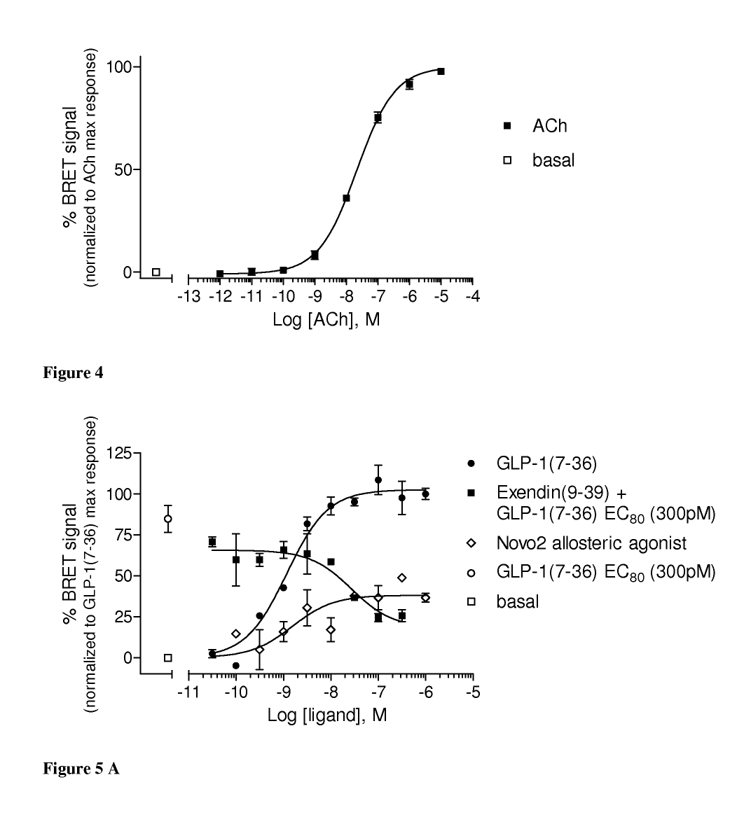

Data from FIG. 4 were obtained conforming to the two-addition protocol described in Example 35, but, unlike in any other example in this patent that were done in stable cell lines, this experiment was performed in transiently transfected HEK cells. Specifically, 1.5 μg of M₄R-GLG2 DNA and 0.25 μg FP-Gγ9 DNA were transiently transfected into HEK cells using Optifect (Invitrogen) transfection reagent following the manufacturer's instructions. 24 h post-transfection, 25,000 cells were seeded into black 384-well plates and left for another 24 h to adhere. On the day of the experiments, after the 30 minutes starvation in 30 μL HBSS++, 15 μl of 24 μM coelenterazine H was added to each well (final coelenterazine H concentration: 4.5 μM) and the cell-plate was inserted into the FLIPR tetra reader, together with the two compound plates. Exposure time in both channels was set to 2 seconds and a two-addition protocol with a read interval of 4.3 seconds was performed. Namely, in the first addition 15 μL of 4 times concentrated antagonist or buffer was added to all wells after the first 20 counts and read until count 70. The second addition took place at count 71, when 20 μL of 4 times concentrated increasing concentrations of acetylcholine, acetylcholine EC₈₀ or buffer was added to each well. The effects were measured until 250 reads. Area under curve of blue/yellow ratio from count 72 to the end of the measurement for the analysis was taken and plotted against acetylcholine/antagonist concentration for the construction of the graph in FIG. 4.

Data on FIG. 5A were obtained by applying a similar one-addition protocol as described for FIG. 3, with the main differences being the final concentration of coelenterazine H of 6 μM, the exposure time in both channels of 3 seconds and the read interval of 6.3 seconds. Further, the addition of the compounds took place after first 30 counts and the effects were measured for another 120 reads. Area under curve of blue/yellow ratio from count 30 to the end of the measurement was taken for the analysis and plotted against GLP-1(7-36)/Exendin(9-39)/Novo 2 concentrations in the graph in FIG. 5A.

Data on FIG. 5B were obtained by applying the two-addition protocol as described above with respect to FIG. 4, with the main differences being the final concentration of coelenterazine H of 6 μM and the presence of 0.2% BSA in the HBSS++ buffer for the dilution of GIP and [Pro³]-GIP. Further, the first addition of the antagonist [Pro³]-GIP, GIP(6-30) or buffer took place after first 2 reads and the second addition of GIP EC₈₀, increasing concentrations of GIP or buffer after the 28^{th} read. The effects were measured for another 150 reads after the second addition. Area under curve of blue/yellow ratio from count 29 to the end of the measurement was taken for the analysis and plotted against GIP/[Pro³]-GIP/GIP(6-30) concentrations for the construction of the graph in FIG. 5B.

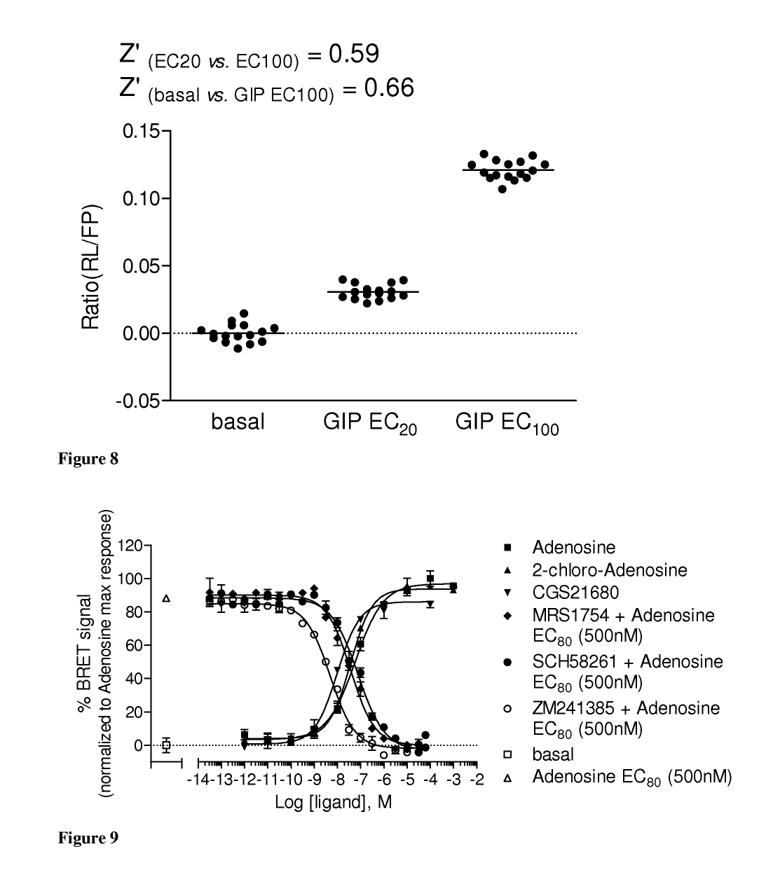

Z′-factors of 0.59 (EC₂₀/EC₁₀₀) and 0.66 (basal/EC₁₀₀) were determined in the same way for this particular stable cell line. These Z′-factor values demonstrate that the method of the invention is suitable for use in HTS (FIG. 8).

Data on FIG. 5C were obtained by applying a two-addition protocol as described above with respect to FIG. 4, with the main differences being the final concentration of coelenterazine H of 6 μM, the exposure time in both channels of 2.5 seconds and the read interval of 5.3 seconds. Further, the first addition of buffer or antagonist SCH442416 was added after the first 15 reads and the second addition of NECA EC₈₀, increasing concentrations of NECA or buffer were added after the 61^{st} read and the effects of the compounds were measured for another 120 reads. Area under the curve of blue/yellow ratio from count 62 until the end of the measurement was taken for the analysis and plotted against NECA/SCH442416 concentrations in the graph of FIG. 5C.

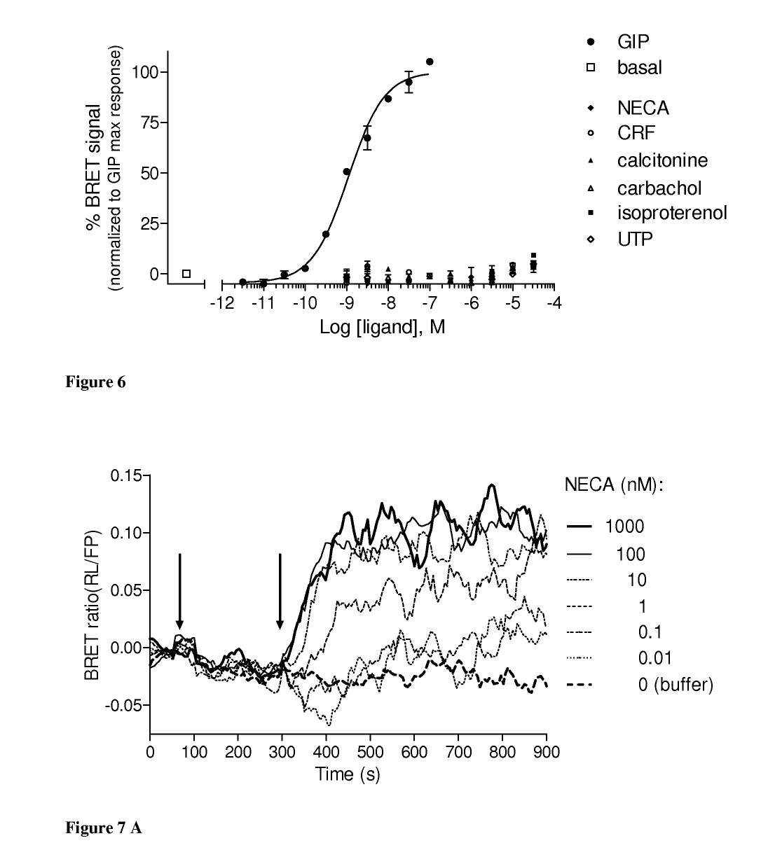

Data on FIG. 6 were obtained by applying a two-addition protocol as described above with respect to FIG. 4, with the main differences being the final concentration of coelenterazine H of 6 μM, the exposure time in both channels of 1.5 seconds and the read interval of 3.3 seconds, as well as the presence of 0.2% BSA in the HBSS++ buffer for diluting peptidic ligands. Further, the first addition of buffer took place 20 reads after the beginning of the measurement and the second addition of buffer or increasing concentrations of GIP, NECA, CRF, calcitonine, carbachol, isoproterenol and UTP was added after the 31^{st} read. The effects of the ligands were read for another 180 reads. Area under curve of blue/yellow ratio from count 31 to the end of the measurement was taken for the analysis and plotted against ligand concentration for the construction of the graph in FIG. 6.

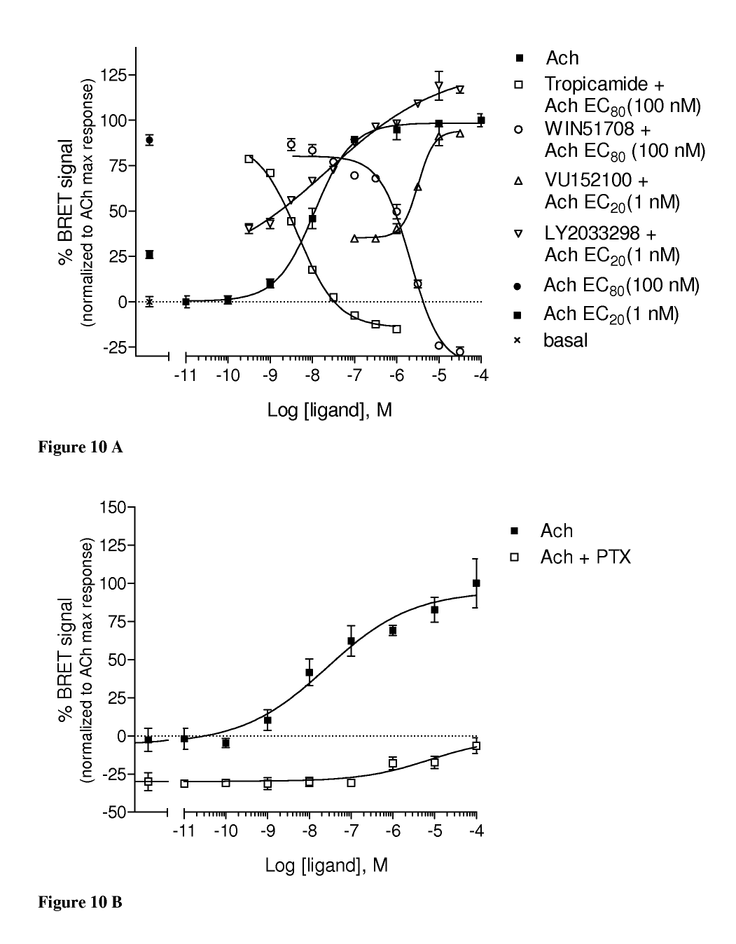

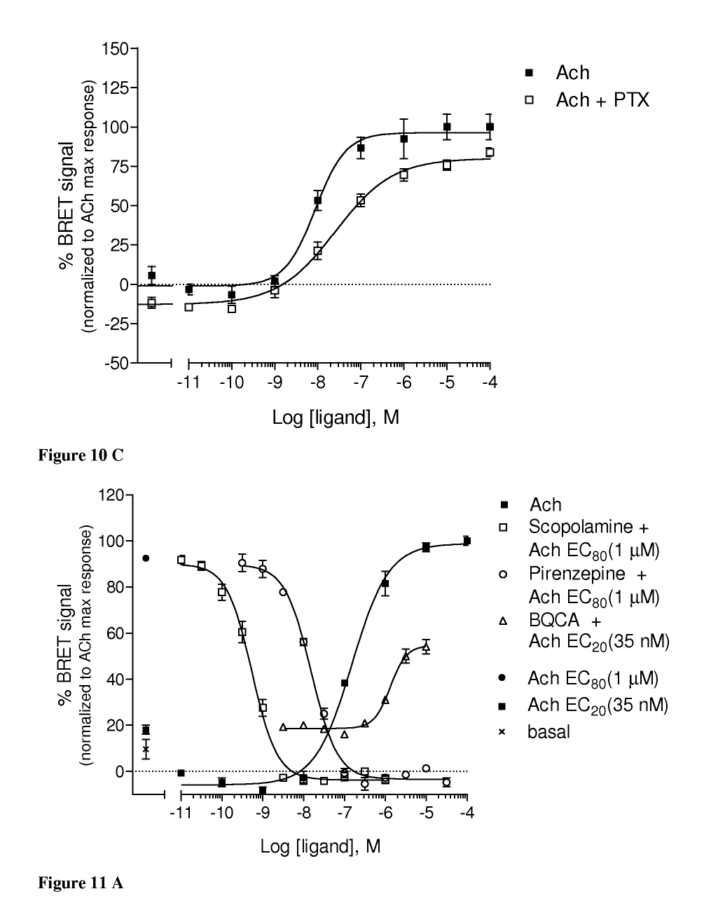

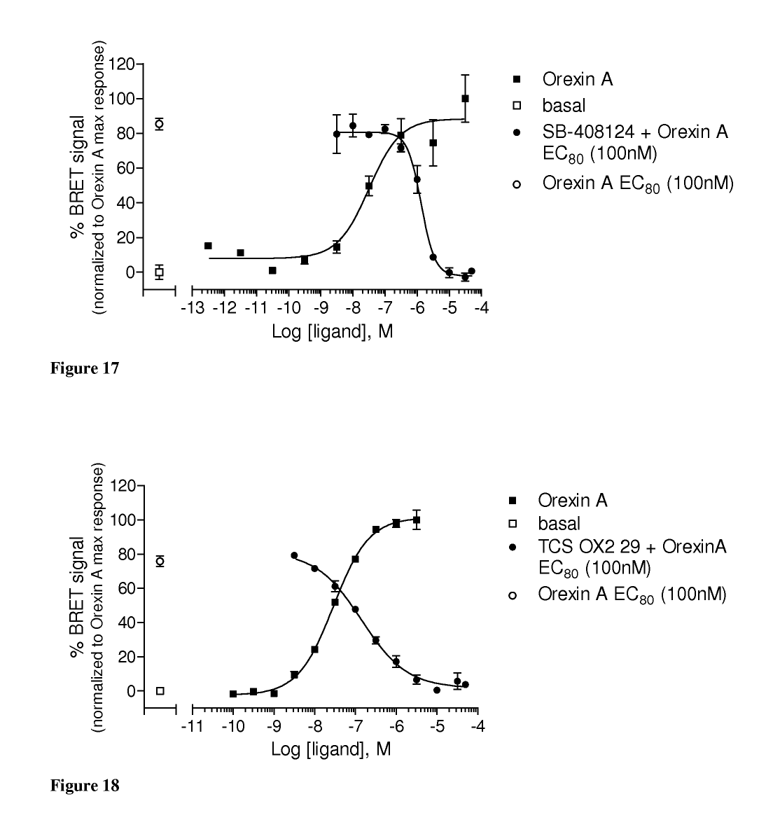

The constructs of Examples 35-42 as indicated in Table 5 above were made using the same general procedure as described above for Examples 3-12. Cells lines were prepared as described above (Examples 13-34). Data were collected using the screening protocol as described above with respect to FIG. 3 and in accordance with the general assay protocol of Example 35. As applicable, the one-addition (FIGS. 10B, 10C, 11B, 13, 14, 19 and 20) or two-addition protocols (FIGS. 9, 10A, 11A, 12, 15, 16, 17 and 18) were used.

The results can be seen in FIGS. 9-18. These figures show that the present invention is useful for screening different types of modulators of different types of GPCRs. Furthermore, the screening method of the invention is suitable for monitoring live cells and on-line, during and immediately after administration of test compounds. A very high throughput is achieved due to the high read frequency and due to the fact that each sample is monitored individually at a high frequency.

As the further examples below will show, these advantages are combined with the surprising findings that false positive hits occurring with state of the art calcium screening assays are avoided. Most surprisingly, allosteric modulators that are not identified by the state of the art screening methods are found by the method of the invention.

### Examples 43

### HTS Compatibility of Cells Expressing Different Gγ Constructs in Accordance with the Invention

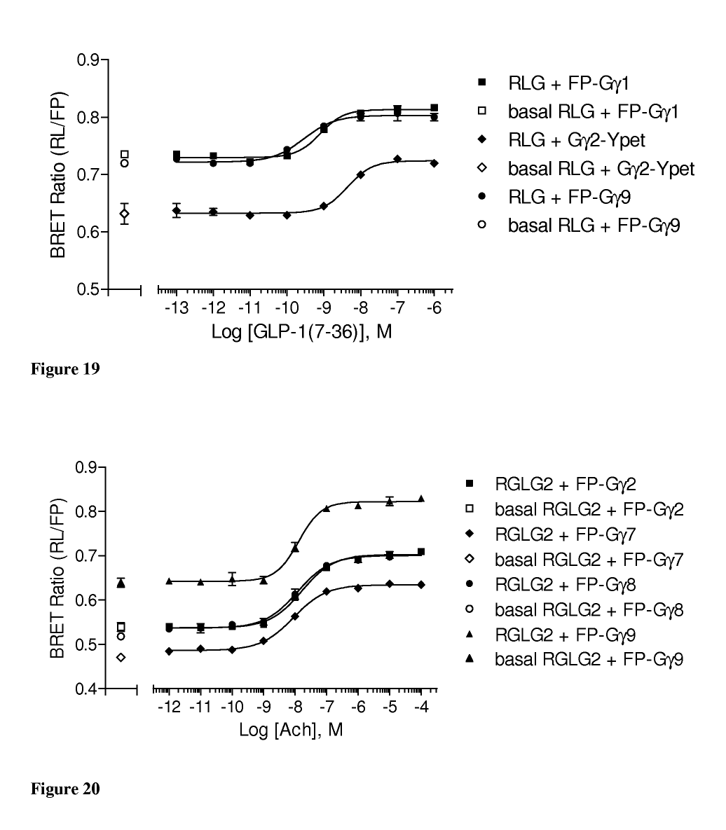

The compatibility of the various Gγ-constructs with HTS is shown in Tables 7 and 8 below. See also FIGS. 19 and 20, where increasing concentrations of agonists were used for testing some of the G-constructs as shown in Table 7.

For assessing HTS compatibility, HEK cells were transiently transfected with different receptor and G-construct combinations according to the protocol as described with respect to FIG. 4 in Example 35 above.

In accordance with Tables 7 and 8, the transiently transfected cells were exposed to the natural ligand of the respective receptor construct and activation of the receptor is measured, allowing determination of window and EC50. Exemplary results of these latter experiments are shown in FIGS. 19 (GIP-R) and 20 (muscarinic M4 receptor).

In Tables 7 and 8, the signs “+” and “++” indicate compatibility or suitability with HTS, whereas “-” indicates absence of compatibility. Empty fields represent combinations that were not tested.

The sequences of the various Gγ-proteins used in the constructs are listed in Table 9 further below.

TABLE 7
Testing HTS compatibility of cells coexpressing different G-gamma
protein constructs and GLP1R-L-G (Example 3), M4R-GLG2
(Example 12) or GIPR-L-G (Example 4) constructs.

| | GLP1R-L-G | | M4R-GLG2 | | GIPR-L-G | |
|---|---|---|---|---|---|---|
| | EC50 | HTS | EC50 | HTS | EC50 | HTS |
| Gγ construct | (GLP-1) | compat. | (ACh) | compat. | (GIP) | compat. |
| Gγ1-FP | 1.1 nM | − | | − | | |
| FP-Gγ1 | 1.1 nM | ++ | 9.4 nM | + | | |
| Gγ2-FP | 3.0 nM | ++ | | − | | |
| FP-Gγ2 | 0.7 nM | + | 12 nM | ++ | | |
| Gγ3-FP | 1.5 nM | + | | − | | |
| FP-Gγ3 | 0.3 nM | − | 17 nM | ++ | | |
| Gγ4-FP | 4.3 nM | − | | − | | |
| FP-Gγ4 | 1.1 nM | − | 9.6 nM | + | | |
| Gγ5-FP | 6.6 nM | + | | − | 5.5 nM | ++ |
| FP-Gγ5 | 0.1 nM | − | 6.6 nM | + | | − |
| Gγ7-FP | 5.2 nM | − | | − | | |
| FP-Gγ7 | 0.4 nM | − | 6.4 nM | ++ | | |
| Gγ8-FP | 2.3 nM | + | | − | | |
| FP-Gγ8 | 1.4 nM | − | 9 nM | ++ | | |
| Gγ9-FP | 1.7 nM | + | | − | 1.4 nM | + |
| FP-Gγ9 | 0.4 nM | ++ | 7.6 nM | ++ | 0.3 nM | ++ |
| Gγ10-FP | 9.2 nM | − | | − | 8.9 nM | + |
| FP-Gγ10 | 0.3 nM | − | 8.3 nM | ++ | | − |
| Gγ11-FP | 3.1 nM | − | | − | 1.6 nM | − |
| FP-Gγ11 | 0.7 nM | + | 9.1 nM | + | 0.4 nM | + |
| Gγ12-FP | 3.1 nM | − | | − | | |
| FP-Gγ12 | 0.3 nM | − | 6.2 nM | ++ | | |
| Gγ13-FP | 1.0 nM | + | | − | 0.6 nM | + |
| FP-Gγ13 | 0.4 nM | − | 8.4 nM | + | 0.3 nM | − |

compat. = compatibility

As can be seen from Tables 7 and 8, suitable combinations of receptor constructs and Gγ-constructs can be determined and selected by the skilled person. According to an embodiment of the screening method of the invention, cells express a respective construct further express a suitable Gγ-construct. Cells expressing a combination of any one the receptors listed in Tables 7 and 8 with Gγ-constructs yielding a “+” or “++” are particularly preferred in accordance with the invention. For example, in accordance with this embodiment, cells expressing GLP1R-RLG preferably coexpress any one selected from FP-Gγ1, Gγ2-FP, FP-Gγ2, Gγ3-FP, Gγ5-FP, Gγ8-FP, Gγ9-F, FP-Gγ9, FP-Gγ11, Gγ13-FP. Similar preferred embodiments for other receptor constructs can be derived from other columns in Tables 7 and 8. Interestingly, with respect to the cells expressing M4R(GLG-2), it can be seen that cells expressing any Gγ₁₋₁₃-FP construct are less preferred, which the inverse (FP-Gγ, the FP at the N-terminus of Gγ) is preferred.

According to an embodiment, Gγ-constructs containing Gγ9 are particularly preferred, including cells coexpressing any receptor construct and a Gγ9-construct. FP-Gγ9 or, according to an alternative embodiment, L-Gγ9 (L=luminescent protein) is particularly preferred.

TABLE 8
Testing HTS compatibility of cells coexpressing different Gγ protein constructs and
A2AR (Example 8), GCGR (Example 39), GHSR (Example 37) and M1R (Example 35).

| | A2AR-RGL | | GCGR-RLG | | GHSR-RLG | | M1R-GLG3 | |
|---|---|---|---|---|---|---|---|---|
| Gγ | | HTS | | HTS | | HTS | | HTS |
| construct | EC50(1) | compat. | EC50(2) | compat. | EC50(3) | compat. | EC50(4) | compat. |
| Gγ1-FP | | | | − | | − | | |
| FP-Gγ1 | | | 19 nM | + | | | 175 nM | ++ |
| Gγ2-FP | | | | − | 0.7 nM | ++ | | |
| FP-Gγ2 | | | | − | | − | 29 nM | ++ |
| Gγ3-FP | | | 9.1 nM | + | 4 nM | ++ | | |
| FP-Gγ3 | | | | − | | − | 35 nM | ++ |
| Gγ4-FP | | | 65 nM | + | | − | | |
| FP-Gγ4 | | | | − | | | 50 nM | ++ |
| Gγ5-FP | | − | | − | | − | | |
| FP-Gγ5 | 933 nM | + | | − | | − | 25 nM | ++ |
| Gγ7-FP | | | 17 nM | ++ | | − | | |
| FP-Gγ7 | | | | − | | | 26 nM | ++ |
| Gγ8-FP | | | | − | | − | | |
| FP-Gγ8 | | | | − | | − | 29 nM | ++ |
| Gγ9-FP | | − | | − | | − | | |
| FP-Gγ9 | 320 nM | ++ | 7.5 nM | ++ | 1.9 nM | ++ | 48 nM | ++ |
| Gγ10-FP | | − | | − | | − | | |
| FP-Gγ10 | 824 nM | + | | − | | − | 59 nM | ++ |
| Gγ11-FP | | − | | − | | − | | |
| FP-Gγ11 | 120 nM | + | 4.6 nM | − | | − | 70 nM | ++ |
| Gγ12-FP | | | 3.7 nM | − | | − | | |
| FP-Gγ12 | | | | − | | | 21 nM | ++ |
| Gγ13-FP | | − | | − | | − | | |
| FP-Gγ13 | 404 nM | + | | − | | | 15 nM | ++ |

(1) Adenosine (nM);
(2) Glucagon;
(3) Ghrelin;
(4) Acetylcholin, all in nM.

TABLE 9
Gγ-sequences of the various Gγ-constructs as shown in Tables 7 and 8 below:

| | Gγ protein | | | | | |
|---|---|---|---|---|---|---|
| | 1 | 2 | 3 | 4 | 5 | 7 |
| Nucleotide | NM_021955 | NM_053064 | NM_012202 | NM_004485 | NM_005274 | NM_052847 |
| accession | | | | | | |
| number | | | | | | |
| SEQ ID | 102 | 103 | 104 | 105 | 106 | 107 |
| NO: | | | | | | |

| | Gγ protein | | | | | |
|---|---|---|---|---|---|---|
| | 8 | 9 | 10 | 11 | 12 | 13 |
| Nucleotide | NM_033258 | NM_031498 | NM_001017998 | NM_004126 | NM_018841 | NM_016541 |
| accession | | | | | | |
| number | | | | | | |
| SEQ ID | 108 | (x) | 109 | 110 | 111 | 112 |
| NO: | | | | | | |

The various constructs in Tables 7 and 8 are obtained by fusing the nucleotide sequences encoding the Gγ proteins of SEQ ID NO: 102-112 (Table 9) to the fluorescent protein (FP) of SEQ ID NO: 2 at its C— (Gγ-FP) or N-terminal (FP-Gγ) end, respectively. (x) See protein sequences SEQ ID NO: 3 and 5 with constructs of FP-Gγ9 and Gγ9-FP, respectively, from which the sequence of Gγ9 can be derived.

### Examples 44 and 45

### Comparison of Screening Method of the Invention with Calcium Based Screening Methods

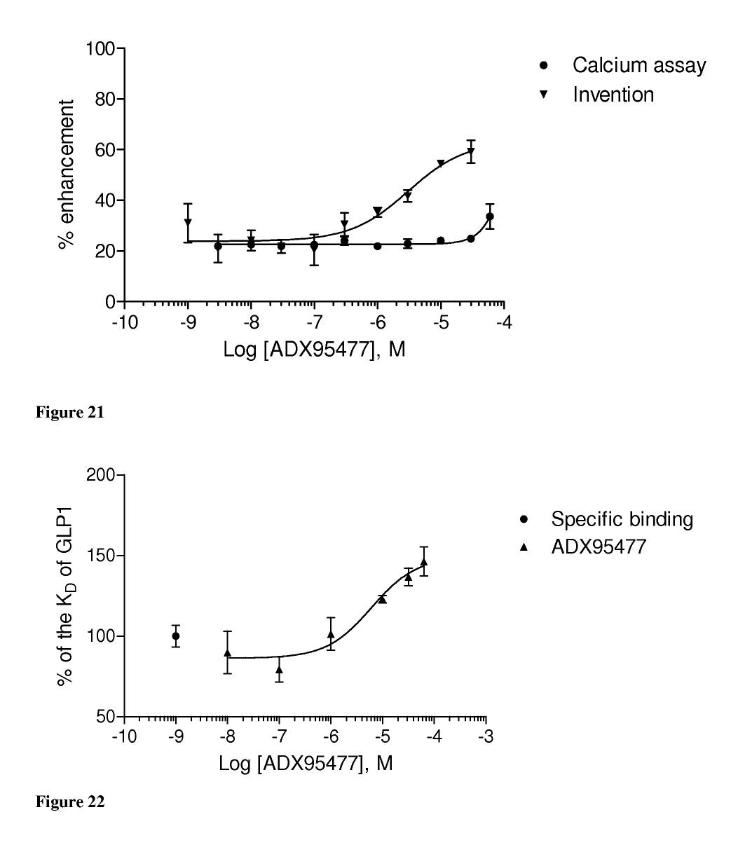

Example 44 FIGS. 21 and 22 described above illustrate with the example of a compound ADX95477 that the screening method of the invention allows identification of active agents that are not found or detected by prior art calcium assay, measuring intracellular calcium flux using Fluo-4. FIG. 21 shows detection of activity by the method of the invention using cells recombinantly expressing a GLP-1R-construct in accordance with the invention. As can be seen, activity is detected at relatively low concentrations already, whereas the calcium assay has a small, possibly un-noted or non-significant response at highest concentration of ADX95477 tested only. In FIG. 22 the activity of the compound is further analysed by checking the effect of the candidate agent on binding of a labelled variant of the natural ligand GLP-1. It is found that ADX95477 is a PAM increasing the affinity and thereby the effect of GLP-1 on the receptor.

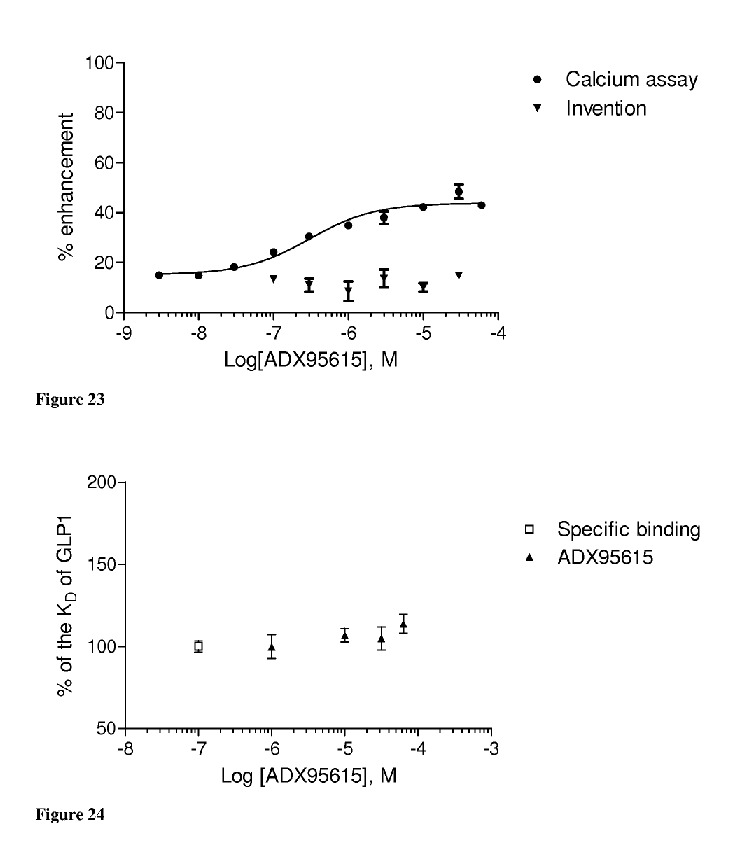

The result of Example 45 is shown in FIGS. 23 and 24, which illustrate with the example of a compound ADX95615 that the screening method of the invention does not identify compounds as “hits” that appear as (false) positives in by prior art calcium assays using Fluo-4. FIG. 23 shows absence of a detection signal with the assay of the invention whereas the calcium assay finds a hit. When checking the effect on affinity of ADX95615 of labelled GLP1 to the GLP-1R, it is found that the compound has no effect and appeared thus a false positive hit in the calcium assay (FIG. 24). This was confirmed in further analysis.

### Example 46

### False Positives of Calcium Based Assay Compared to Assay of the Invention

The assay of the present invention was compared with a state of the art calcium based assay. Activities of different compounds tested alone, in presence of EC₂₀ value of Ach and in presence of EC₈₀ of Ach are measured. In calcium-based assay, a calcium flux is measured in cells co-expressing recombinant M₄R and modified Gαq. In the assay in accordance with the present invention, BRET in measured in cells co-expressing a chimeric M₄R according to RGLG-2 (construct and cell-line from FIG. 2C, Table 6) and FP-Gγ9 (as described in FIG. 10A). The data represent mean values and they are normalized to the maximal effect of the orthosteric agonist after the subtraction of the basal values.

TABLE 10
Occurrence of False positives in state of the art Calcium-based screening
assays compared to the assay in accordance with the invention
(Example 33).

| activity @ 30uM or | | | | | | |
|---|---|---|---|---|---|---|
| 30 ug/ml | | | | | | |
| (except * @ 3uM or | Calcium-based assay | | | Invention assay | | |
| 3 ug/ml) | % stim | % enh | % inh | % stim | % enh | % inh |
| (±)-Blebbistatin | 130.0 | 115.1 | −46.9 | −7.2 | −2.4 | 13.1 |
| (R)-(+)-Bay K 8644 | 26.8 | 77.8 | −66.5 | −8.2 | 4.7 | 11.4 |
| (S)-(−)-Bay K 8644 | 16.1 | 59.8 | −65.0 | −8.0 | −3.6 | 14.6 |
| 2-APB | 3.7 | 4.8 | 30.3 | −4.7 | 6.3 | 10.5 |
| 4-Hydroxynonenal | 1.4 | −7.3 | 118.9 | −14.9 | −3.5 | 29.9 |
| AG 10 | 2.3 | 3.8 | 41.6 | −13.0 | −0.1 | 8.6 |
| BPIPP | 18.1 | 62.6 | −29.2 | 1.3 | 8.3 | 1.5 |
| Brefeldin A | 5.2 | 32.3 | −19.5 | −2.5 | −1.9 | 12.7 |
| D15 | 1.8 | −3.0 | 36.5 | −2.1 | 2.2 | 7.8 |
| Dynamin Inhibitor I, | 13.4 | 92.3 | −73.7 | −8.0 | 23.9 | −3.5 |
| FPL 64176 | 48.9 | 59.7 | −66.6 | −8.4 | 7.5 | 10.0 |
| FSK | 57.8 | 58.3 | −22.4 | −5.0 | −3.6 | 11.8 |
| Gallein | 11.7 | 42.9 | −22.2 | 3.9 | 5.1 | 5.3 |
| GF 109203X | 154.3 | 84.3 | −10.0 | 2.2 | 7.4 | 29.5 |
| G-Protein antagonist | 7.2 | −2.5 | 38.3 | −1.2 | 3.6 | 8.1 |
| GRK2i | 5.5 | −2.1 | 35.0 | −0.5 | 2.5 | 7.0 |
| GW 5074 | 879.7 | 151.4 | −23.8 | −6.8 | 2.8 | 13.5 |
| Ionomycin calcium | 488.7 | 58.8* | 112.2 | −11.8 | −2.5 | 14.6 |
| salt | | | | | | |
| KN-62 | 8.1* | 39.4* | | −0.8* | −1.2* | 15.8* |
| Monastrol | 4.1 | 44.7 | −16.7 | −3.0 | −0.1 | 7.0 |
| N-Ethylmaleimide | 3.8 | 0.2 | 96.9 | −3.4 | 12.4 | 16.5 |
| Nifedipine | −0.9 | 8.7 | 34.8 | −15.8 | 5.3 | 23.2 |
| Oligomycin | 3.3 | 63.8 | −21.6 | 1.8 | 3.3 | 8.5 |
| SIN-1 chloride | 5.1 | 19.4 | 17.1 | −1.3 | 3.6 | 6.6 |
| Thapsigargin | 129.5 | 49.8 | 11.6 | −0.7 | −4.2 | 10.8 |

As can be seen from Table 10, the screening assay of the invention does not indicate the presence of a hit in the case of a large number of compounds appearing as (false) positives in a state of the art calcium assay. In conclusion, the screening kethod of the invention is more reliable and more specific that the state of the art calcium assay.

### Example 47

### Assessment of Specificity of the Assay of the Invention

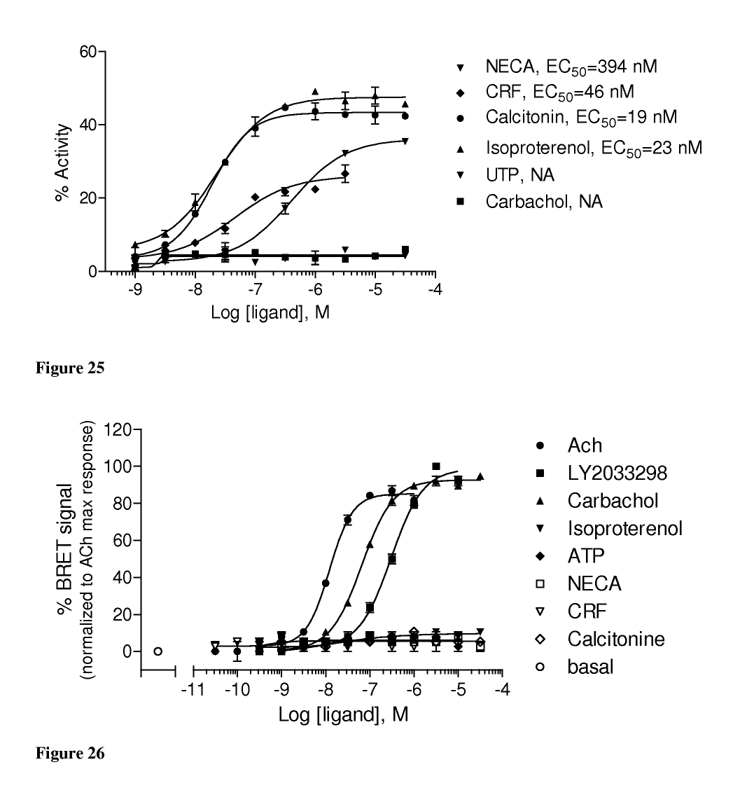

As shown in FIG. 25, non-transfected HEK cells are activated by any one of NECA, Corticotropin releasing factor, calcitonin, isoproterenol, UTP and carbachol, aswas determined by measuring cAMP accumulation in such cells following exposure to any one of these compounds. On the other hand, the same cells, transfected to co-express the M₄R-G-LG and FP Gγ9 constructs, do not show any response when tested in the assay of the invention. Only when exposed to reported modulators is an activation of the target GPCR (here M4R) seen. This demonstrates the specificity of the assay of the invention, which is robust with respect to receptor cross-talk and interference.

1. A method of high-throughput screening for agents, which are capable of affecting the activity of a G Protein-Coupled Receptor (GPCR), said method comprising the steps:
exposing a sample of cells to a candidate agent;
conducting, in an automated manner, measurements of light emitted from the sample;
wherein said cells express at least a recombinant nucleotide sequence encoding a chimeric protein (a) comprising a GPCR fused to a Gα subunit, wherein, in said cells, said chimeric protein (a) is either further fused to a luminescent protein or is connected to a fluorescent entity, said cells also expressing a recombinant nucleotide sequence encoding a protein (b) comprising a Gγ and/or a Gβ subunit, wherein, in said cells, said protein (b) is either connected to a fluorescent entity or further fused to a luminescent protein;
with the provisos that if (a) comprises a luminescent protein, the protein (b) comprises a fluorescent entity, and if protein (b) comprises a luminescent protein, (a) comprises a fluorescent entity, and that the luminescent protein or fluorescent entity of chimeric protein (a) emits light of a different wavelength than the luminescent protein or fluorescent entity of the protein (b);
wherein said protein (a) and said protein (b) can exist in said cells in a first state, where there is no or little energy transfer, and a second state, where there is a substantial energy transfer from the luminescent protein or fluorescent entity of chimeric protein (a) to the luminescent protein or fluorescent entity of protein (b), or vice versa, wherein said energy transfer affects the quantity of measurable light emitted by said fluorescent protein(s) and/or said luminescent protein;
wherein an activation of said GPCR by said candidate agent determines if (a) and (b) are in said first or second state and therefore the quantities of light emitted from (a) and/or (b)
wherein said candidate agent is considered an active agent if it has a detectable effect on the quantity of light units emitted by said protein (a) and/or said protein (b) compared to a sample devoid of said candidate agent.

2. The method of claim 1, wherein said fluorescent entity is a fluorescent protein, and wherein said cells express at least a recombinant nucleotide sequence encoding a chimeric protein (a) comprising an amino acid sequence of a GPCR fused to, in any order, the amino acid sequence of a Gα subunit and also to an amino acid sequence of one selected from a luminescent protein and a fluorescent protein, said cells also expressing a recombinant nucleotide sequence encoding a chimeric protein (b) comprising a Gγ and/or a Gβ subunit fused to one selected from a luminescent protein and a fluorescent protein, with the provisos that if (a) comprises a luminescent protein, the chimeric protein (b) comprises a fluorescent protein, and if chimeric protein (b) comprises a luminescent protein, (a) comprises a fluorescent protein, and that the luminescent protein or fluorescent protein of chimeric protein (a) emits light of a different wavelength than the luminescent protein or fluorescent protein of the chimeric protein (b).

3. The method of claim 1, wherein said step of conducting measurements of light comprises: conducting, in an automated manner, a series of repeated measurements of light emitted from the sample, wherein said measurements are repeated within determined time intervals of 0.4 seconds to 10 minutes over a total period of up to 30 minutes;

4. The method of claim 1, wherein said sample of cells is provided in a well of a microtiter plate comprising a plurality of further wells with further samples, and wherein a plurality of different candidate agents and/or candidate agents at different concentrations are comprised in an individual microtiter plate.

5. The method of claim 3, wherein, in each of the repeated measurements light emission of all samples in the wells of an entire microtiter plate is measured at the determined time intervals simultaneously.

6. The method of claim 1, wherein an activity of said GPCR is any one selected from the group consisting of receptor activating, inhibiting, modulating and/or cell signalling activity.

7. The method of claim 4, wherein said microtiter plate comprises 384 wells or more.

8. The method of claim 5, wherein, in the step of simultaneously measuring light emission, light emission is determined by taking at least one photograph of said entire microtiter plate.

9. The method of claim 1, wherein a read frequency of a cell plate comprising 384 samples or more, that is, the frequency at which light emission from any individual samples can be measured again following the measurement of all samples, is greater than 0.5 Hz.

10. The method of claim 1, wherein said method further comprises the step of further exposing the sample of cells to an orthosterically binding control agent, wherein said orthosteric agent is added at a concentration selected to produce a specific controlled activity on the receptor.

11. The method of claim 10, wherein the control agent is added at an EC value selected from the values covered by the range EC5-EC100, preferably EC10-EC90.

12. The method of claim 1, wherein said nucleotide sequence encoding said chimeric protein (a), encodes, in this order from 3′ to 5′, one selected from:
i) GPCR—Gα—luminescent protein;
ii) GPCR—Gα—fluorescent protein;
iii) GPCR—luminescent protein—Ga;
iv) GPCR—fluorescent protein—Ga;
v) GPCR—split Gα part I—luminescent protein—split Gα part II;
vi) GPCR—split Gα part I—fluorescent protein—split Gα part II;
wherein amino acid moieties and/or sequences having or, independently, not having further functionalities may be independently provided terminally and/or in positions indicated with “-”.

13. The method of claim 1, wherein said nucleotide sequence encoding said protein (b), encodes, in this order from 3′ to 5′, one selected from:
vii) Gγ—luminescent protein;
viii) Gγ—fluorescent protein;
ix) luminescent protein—Gγ;
x) fluorescent protein—Gγ;
wherein amino acid moieties and/or sequences having or, independently, not having further functionalities may be independently provided terminally and/or in positions indicated with “-”.

14. (canceled)

15. (canceled)

16. The method of claim 1, wherein said fluorescent entity is a fluorescent protein comprising at least one selected from (i) an amino acid sequence encoded by the nucleotide sequence according to SEQ. ID. NO.: 2, and (ii) an amino acid sequence having at least 90% identity with the amino acid sequence of (i).

17. The method of claim 1, wherein said fluorescent entity is a mutant Yellow Fluorescent Protein (YFP), said mutant YFP comprising, if aligned with green-FP, one, several or all mutations selected from the group consisting of F46L, 147L, F64L, R79K, M153T, V163A, S175G, S208F, S208F, V224L, H231E and D234N.

18. The method of claim 1, wherein said luminescent protein comprises at least one selected from (i) an amino acid sequence encoded by the nucleotide sequence according to (i) SEQ. ID. NO.: 1, and (ii) an amino acid sequence having at least 90% identity with the amino acid sequence of (i).

19. The method of claim 1, wherein said chimeric protein (a) comprises at least one selected from (i) an amino acid sequence selected from any one of SEQ. ID. NO.:8, 10, 12, 14, 16, 18, 20, 22, 24, 84, 87, 89, 91, 93, 95, 97, 99, and 101; (ii) an amino acid sequence having at least 70% identity with any one sequence listed under (i).

20. The method of claim 1, wherein said protein (b) is a chimeric protein (b) comprising at least one selected from (i) an amino acid sequence selected from any one of SEQ. ID. NO.: 4 and 6, or a Gγ sequence selected from any one of any one of SEQ. ID. NO.: 102-112, or the Gγ9 sequence contained in any one of SEQ. ID. NO.:4 and 6; and (ii) an amino acid sequence having at least 70% identity with any one of SEQ. ID. NO.: 4 and 6.

21. (canceled)

22. A nucleotide sequence encoding a chimeric protein (a), said nucleotide sequence comprising a GPCR, a Gα subunit, and one selected from a luminescent protein and a fluorescent protein.

23. (canceled)

24. (canceled)

25. A chimeric protein (a) comprising a GPCR fused to a Gα subunit and either also fused to a luminescent protein or covalently connected to a fluorescent entity.

26. Cells comprising a chimeric protein (a) comprising a GPCR fused to a Gα subunit and either also fused to a luminescent protein or connected to a fluorescent entity, and, optionally, expressing a recombinant nucleotide sequence encoding a protein (b) comprising a Gγ and/or a Gβ subunit, wherein, in said cells, said protein (b) is either covalently connected to a fluorescent entity or further fused to a luminescent protein, with the provisos that if (a) comprises a luminescent protein, the protein (b) comprises a fluorescent entity, and if protein (b) comprises a luminescent protein, (a) comprises a fluorescent entity, and that the luminescent protein or fluorescent entity of chimeric protein (a) emits light of a different wavelength than the luminescent protein or fluorescent entity of the protein (b).

27. (canceled)
